(19) United States
(12) Patent Application Publication
(10) Pub. No.: US 2024/0117363 A1
(43) Pub. Date: Apr. 11, 2024

(54) PRODUCTION OF UNNATURAL NUCLEOTIDES USING A CRISPR/CAS9 SYSTEM

(71) Applicant: The Scripps Research Institute, La Jolla, CA (US)

(72) Inventors: Floyd E. ROMESBERG, La Jolla, CA (US); Brian LAMB, San Diego, CA (US); Yorke ZHANG, San Diego, CA (US)

(21) Appl. No.: 18/228,251

(22) Filed: Jul. 31, 2023

Related U.S. Application Data
(62) Division of application No. 16/063,107, filed on Jun. 15, 2018, now Pat. No. 11,761,007.
(60) Provisional application No. 62/269,890, filed on Dec. 18, 2015.

(51) Int. Cl. C12N 15/63; C12N 5/10; C12N 9/22; C12N 15/11; C12N 15/70

(52) U.S. Cl. CPC C12N 15/63; C12N 5/10; C12N 9/22; C12N 15/11; C12N 15/70; C12N 2310/20; C12N 2310/3519

(57) ABSTRACT

Disclosed herein are methods, cells, engineered microorganisms, and kits for increased production of a nucleic acid molecule that comprises an unnatural nucleotide.

### CROSS-REFERENCE

This application is a divisional of U.S. application Ser. No. 16/063,107, filed Jun. 15, 2018, which is a U.S. National Stage entry of International Application No. PCT/US2016/067353, filed Dec. 16, 2016, which claims the benefit of U.S. Provisional Application No. 62/269,890, filed on Dec. 18, 2015, both of which are incorporated herein by reference in their entireties.

### STATEMENT AS TO FEDERALLY SPONSORED RESEARCH

This invention was made with government support under GM060005 awarded by The National Institutes of Health. The government has certain rights in this invention.

### SEQUENCE LISTING

The instant application contains a Sequence Listing which has been submitted electronically in XML format and is hereby incorporated by reference in its entirety. Said XML copy, created on Nov. 22, 2023, is named “01183-5003-01US-SYN.xml” and is 184,072 bytes in size.

### BACKGROUND OF THE INVENTION

The ability to sequence-specifically synthesize/amplify oligonucleotides (DNA or RNA) with polymerases, for example by PCR or isothermal amplification systems (e.g., transcription with T7 RNA polymerase), has revolutionized biotechnology. In addition to all of the potential applications in nanotechnology, this has enabled a diverse range of new technologies such as the in vitro evolution via SELEX(Systematic Evolution of Ligands by Exponential Enrichment) of RNA and DNA aptamers and enzymes. See, for example, Oliphant AR, Brandl C J & Struhl K (1989), Defining the sequence specificity of DNA-binding proteins by selecting binding sites from random-sequence oligonucleotides: analysis of yeast GCN4 proteins, Mol. Cell Biol., 9:2944-2949; Tuerk C & Gold L (1990), Systematic evolution of ligands by exponential enrichment: RNA ligands to bacteriophage T4 DNA polymerase, Science, 249:505-510; Ellington A D & Szostak J W (1990), In vitro selection of RNA molecules that bind specific ligands, Nature, 346:818-822.

In some aspects, these applications are restricted by the limited chemical/physical diversity present in the natural genetic alphabet (the four natural nucleotides A, C, G, and T in DNA, and the four natural nucleotides A, C, G, and U in RNA). Disclosed herein is a method of generating nucleic acids that contains an expanded genetic alphabet.

### SUMMARY OF THE INVENTION

Described herein, in certain embodiments, are methods, cells, engineered microorganisms, plasmids, and kits for increased production of a nucleic acid molecule that comprises an unnatural nucleotide. In some embodiments, also described herein include methods, cells, engineered microorganisms, plasmids, and kits that utilizes a CRISPR/Cas editing system for increased production of a nucleic acid molecule that comprises an unnatural nucleotide. In some embodiments, further described herein include methods, cells, engineered microorganisms, plasmids, and kits that utilizes a CRISPR/Cas editing system for retention of a nucleic acid molecule that comprises an unnatural nucleotide.

Disclosed herein, in certain embodiments, is an engineered cell comprising: (a) a first nucleic acid molecule encoding a Cas9 polypeptide or variants thereof, (b) a second nucleic acid molecule encoding a single guide RNA (sgRNA) comprising a crRNA-tracrRNA scaffold; and (c) a third nucleic acid molecule comprising an unnatural nucleotide; wherein the first nucleic acid molecule, the second nucleic acid molecule, and the third nucleic acid molecule are encoded in one or more plasmids, and the sgRNA encoded by the second nucleic acid molecule comprises a target motif that recognizes a modification at the unnatural nucleotide position within the third nucleic acid molecule. In some embodiments, the modification at the unnatural nucleotide position within the third nucleic acid molecule generates a modified third nucleic acid molecule. In some embodiments, the modification is a substitution. In some embodiments, the modification is a deletion. In some embodiments, the modification is an insertion. In some embodiments, the sgRNA encoded by the second nucleic acid molecule further comprises a protospacer adjacent motif (PAM) recognition element. In some embodiments, the PAM element is adjacent to the 3′ terminus of the target motif. In some embodiments, the target motif is between 15 to 30 nucleotides in length. In some embodiments, the target motif is about 15, 16, 17, 18, 19, 20, 21, or 22 nucleotides in length. In some embodiments, a nucleotide within the target motif that pairs with the modification at the unnatural nucleotide position within the third nucleic acid molecule is located between 3 to 22, between 5 to 20, between 5 to 18, between 5 to 15, between 5 to 12, or between 5 to 10 nucleotides from the 5′ terminus of PAM. In some embodiments, a nucleotide within the target motif that pairs with the modification at the unnatural nucleotide position within the third nucleic acid molecule is located about 3, 4, 5, 6, 7, 8, 9, 10, 11, 12, 13, 14, or 15 nucleotides from the 5′ terminus of PAM. In some embodiments, the combination of Cas9 polypeptide or variants thereof and sgRNA modulates replication of the modified third nucleic acid molecule. In some embodiments, the combination of Cas9 polypeptide or variants thereof and sgRNA decreases the replication rate of the modified third nucleic acid molecule by about 80%, 85%, 95%, 99%, or higher. In some embodiments, the production of the third nucleic acid molecule in the cell increases by about 50%, 60%, 70%, 80%, 90%, 95%, 96%, 97%, 98%, 99%, or higher. In some embodiments, the Cas9 polypeptide or variants thereof generate a double-stranded break. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

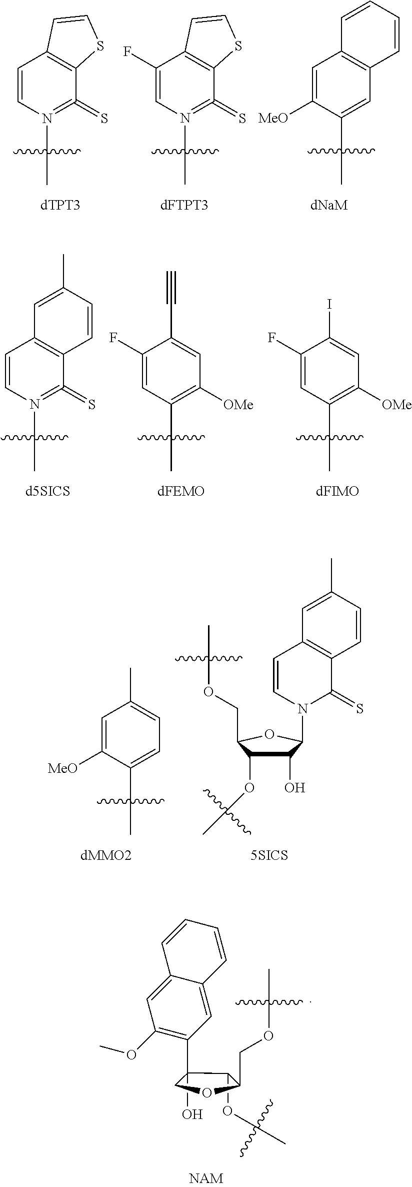

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH2)n O]mCH₃, —O(CH₂)nOCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the sgRNA has less than about 20%, 15%, 10%, 5%, 3%, 1%, or less off-target binding rate. In some embodiments, the cell further comprises an additional nucleic acid molecule that encodes an additional single guide RNA (sgRNA) comprising a crRNA-tracrRNA scaffold. In some embodiments, the third nucleic acid molecule further comprises an additional unnatural nucleotide. In some embodiments, the cell is a prokaryotic cell. In some embodiments, the cell is E. coli. In some embodiments, the cell is a fungal cell. In some embodiments, the cell is a yeast cell. In some embodiments, the cell is a eukaryotic cell. In some embodiments, the cell generates a stable cell line. In some embodiments, disclosed herein is an engineered cell comprising: (a) a first nucleic acid molecule encoding a Cas9 polypeptide or variants thereof, (b) a second nucleic acid molecule encoding two or more single guide RNAs (sgRNAs) wherein each sgRNA comprises a crRNA-tracrRNA scaffold; and (c) a third nucleic acid molecule comprising an unnatural nucleotide; wherein the first nucleic acid molecule, the second nucleic acid molecule, and the third nucleic acid molecule are encoded in one or more plasmids, and each of the sgRNAs encoded by the second nucleic acid molecule comprises a target motif that recognizes a modification at the unnatural nucleotide position within the third nucleic acid molecule.

Disclosed herein, in certain embodiments, is an in vivo method of increasing the production of a nucleic acid molecule containing an unnatural nucleotide, comprising incubating a cell with: (a) a first nucleic acid molecule encoding a Cas9 polypeptide or variants thereof, (b) a second nucleic acid molecule encoding a single guide RNA (sgRNA) comprising a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and sgRNA modulates replication of the modified third nucleic acid molecule to increase the production of the nucleic acid molecule containing an unnatural nucleotide. In some embodiments, the modification is a substitution. In some embodiments, the modification is a deletion. In some embodiments, the modification is an insertion. In some embodiments, the sgRNA encoded by the second nucleic acid molecule comprises a target motif that recognizes a modification at the unnatural nucleotide position within the third nucleic acid molecule. In some embodiments, the sgRNA encoded by the second nucleic acid molecule further comprises a protospacer adjacent motif (PAM) recognition element. In some embodiments, PAM is adjacent to the 3′ terminus of the target motif. In some embodiments, the target motif is between 15 to 30 nucleotides in length. In some embodiments, the target motif is about 15, 16, 17, 18, 19, 20, 21, or 22 nucleotides in length. In some embodiments, a nucleotide within the target motif that pairs with the modification at the unnatural nucleotide position within the third nucleic acid molecule is located between 3 to 22, between 5 to 20, between 5 to 18, between 5 to 15, between 5 to 12, or between 5 to 10 nucleotides from the 5′ terminus of PAM. In some embodiments, a nucleotide within the target motif that pairs with the modification at the unnatural nucleotide position within the third nucleic acid molecule is located about 3, 4, 5, 6, 7, 8, 9, 10, 11, 12, 13, 14, or 15 nucleotides from the 5′ terminus of PAM. In some embodiments, the combination of Cas9 polypeptide or variants thereof and sgRNA modulates replication of the modified third nucleic acid molecule. In some embodiments, the combination of Cas9 polypeptide or variants thereof and sgRNA decreases the replication rate of the modified third nucleic acid molecule by about 80%, 85%, 95%, 99%, or higher. In some embodiments, the production of the third nucleic acid molecule increases by about 50%, 60%, 70%, 80%, 90%, 95%, 96%, 97%, 98%, 99%, or higher. In some embodiments, the Cas9 polypeptide or variants thereof generate a double-stranded break. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

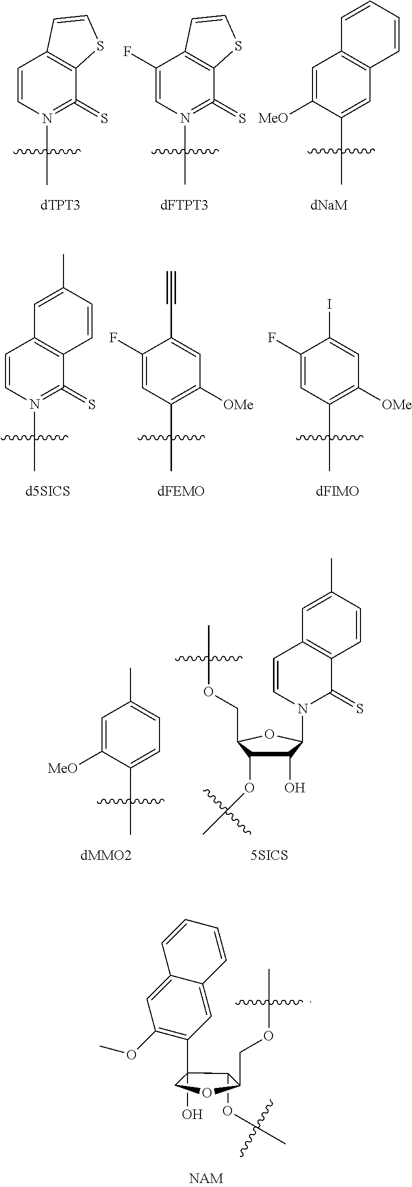

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′-OCH₃, 2′—O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH2)n O]mCH₃, —O(CH₂)nOCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the sgRNA has less than about 20%, 15%, 10%, 5%, 3%, 1%, or less off-target binding rate. In some embodiments, the method further comprises an additional nucleic acid molecule that encodes an additional single guide RNA (sgRNA) comprising a crRNA-tracrRNA scaffold. In some embodiments, the third nucleic acid molecule further comprises an additional unnatural nucleotide. In some embodiments, the first nucleic acid molecule, the second nucleic acid molecule, and the third nucleic acid molecule are encoded in one or more plasmids. In some embodiments, the incubating further comprises a transformation step. In some embodiments, the cell is a prokaryotic cell. In some embodiments, the cell is E. coli. In some embodiments, the cell is a fungal cell. In some embodiments, the cell is a yeast cell. In some embodiments, the cell is a eukaryotic cell. In some embodiments, the cell generates a stable cell line. In some embodiments, is an in vivo method of increasing the production of a nucleic acid molecule containing an unnatural nucleotide, comprising incubating a cell with: (a) a first nucleic acid molecule encoding a Cas9 polypeptide or variants thereof, (b) a second nucleic acid molecule encoding two or more single guide RNAs (sgRNAs) wherein each sgRNA comprises a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and the two or more sgRNAs modulates replication of the modified third nucleic acid molecule to increase the production of the nucleic acid molecule containing an unnatural nucleotide.

Disclosed herein, in certain embodiments, is a nucleic acid molecule containing an unnatural nucleotide produced by a process comprising incubating a cell with: (a) a first nucleic acid molecule encoding a Cas9 polypeptide or variants thereof; (b) a second nucleic acid molecule encoding a single guide RNA (sgRNA) comprising a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and sgRNA modulates replication of the modified third nucleic acid molecule leading to production of the nucleic acid molecule containing an unnatural nucleotide. In some embodiments, the modification is a substitution. In some embodiments, the modification is a deletion. In some embodiments, the modification is an insertion. In some embodiments, the sgRNA encoded by the second nucleic acid molecule comprises a target motif that recognizes a modification at the unnatural nucleotide position within the third nucleic acid molecule. In some embodiments, the sgRNA encoded by the second nucleic acid molecule further comprises a protospacer adjacent motif (PAM) recognition element. In some embodiments, PAM is adjacent to the 3′ terminus of the target motif. In some embodiments, the target motif is between 15 to 30 nucleotides in length. In some embodiments, the target motif is about 15, 16, 17, 18, 19, 20, 21, or 22 nucleotides in length. In some embodiments, a nucleotide within the target motif that pairs with the modification at the unnatural nucleotide position within the third nucleic acid molecule is located between 3 to 22, between 5 to 20, between 5 to 18, between 5 to 15, between 5 to 12, or between 5 to 10 nucleotides from the 5′ terminus of PAM. In some embodiments, a nucleotide within the target motif that pairs with the modification at the unnatural nucleotide position within the third nucleic acid molecule is located about 3, 4, 5, 6, 7, 8, 9, 10, 11, 12, 13, 14, or 15 nucleotides from the 5′ terminus of PAM. In some embodiments, the combination of Cas9 polypeptide or variants thereof and sgRNA modulates replication of the modified third nucleic acid molecule. In some embodiments, the combination of Cas9 polypeptide or variants thereof and sgRNA decreases the replication rate of the modified third nucleic acid molecule by about 80%, 85%, 95%, 99%, or higher. In some embodiments, the production of the third nucleic acid molecule increases by about 50%, 60%, 70%, 80%, 90%, 95%, 96%, 97%, 98%, 99%, or higher. In some embodiments, the Cas9 polypeptide or variants thereof generate a double-stranded break. In some embodiments, the Cas9 polypeptide is a wild-type Cas9. In some embodiments, the unnatural nucleotide comprises an unnatural base selected from the group consisting of 2-aminoadenin-9-yl, 2-aminoadenine, 2-F-adenine, 2-thiouracil, 2-thio-thymine, 2-thiocytosine, 2-propyl and alkyl derivatives of adenine and guanine, 2-amino-adenine, 2-amino-propyl-adenine, 2-aminopyridine, 2-pyridone, 2′-deoxyuridine, 2-amino-2′-deoxyadenosine 3-deazaguanine, 3-deazaadenine, 4-thio-uracil, 4-thio-thymine, uracil-5-yl, hypoxanthin-9-yl (I), 5-methyl-cytosine, 5-hydroxymethyl cytosine, xanthine, hypoxanthine, 5-bromo, and 5-trifiuoromethyl uracils and cytosines; 5-halouracil, 5-halocytosine, 5-propynyl-uracil, 5-propynyl cytosine, 5-uracil, 5-substituted, 5-halo, 5-substituted pyrimidines, 5-hydroxycytosine, 5-bromocytosine, 5-bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 6-alkyl derivatives of adenine and guanine, 6-azapyrimidines, 6-azo-uracil, 6-azo cytosine, azacytosine, 6-azo-thymine, 6-thio-guanine, 7-methylguanine, 7-methyladenine, 7-deazaguanine, 7-deazaguanosine, 7-deaza-adenine, 7-deaza-8-azaguanine, 8-azaguanine, 8-azaadenine, 8-halo, 8-amino, 8-thiol, 8-thioalkyl, and 8-hydroxyl substituted adenines and guanines; N4-ethylcytosine, N-2 substituted purines, N-6 substituted purines, O-6 substituted purines, those that increase the stability of duplex formation, universal nucleic acids, hydrophobic nucleic acids, promiscuous nucleic acids, size-expanded nucleic acids, fluorinated nucleic acids, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido [3′,2′:4,5]pyrrolo [2,3-d]pyrimidin-2-one), 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methythio-N6-isopentenyladeninje, uracil-5oxyacetic acid, wybutoxosine, pseudouracil, queosine, 2-thiocytosine, 5-methyl-2-thiouracil, 2-thiouracil, 4-thiouracil, 5-methyluracil, uracil-5-oxacetic acid methylester, uracil-5-oxacetic acid, 5-methyl-2-thiouracil, 3-(3-amino-3-N-2-carboxypropyl) uracil, (acp3)w, and 2,6-diaminopurine and those in which the purine or pyrimidine base is replaced with a heterocycle. In some embodiments, the unnatural base is selected from the group consisting of

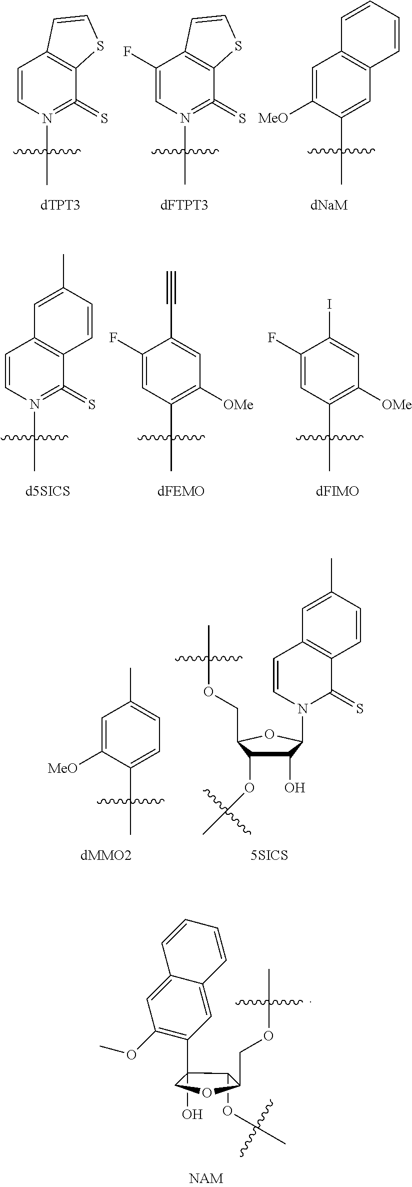

In some embodiments, the unnatural nucleotide further comprises an unnatural sugar moiety. In some embodiments, the unnatural sugar moiety is selected from the group consisting of a modification at the 2′ position: OH; substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂F; O-alkyl, S-alkyl, N-alkyl; O-alkenyl, S-alkenyl, N-alkenyl; O-alkynyl, S-alkynyl, N-alkynyl; O-alkyl-O-alkyl, 2′-F, 2′—OCH₃, 2′-O(CH₂)₂OCH₃ wherein the alkyl, alkenyl and alkynyl may be substituted or unsubstituted C₁-C₁₀, alkyl, C₂-C₁₀ alkenyl, C₂-C₁₀ alkynyl, —O[(CH2)n O]mCH₃, —O(CH₂)nOCH₃, —O(CH₂)n NH₂, —O(CH₂)n CH₃, —O(CH₂)n —ONH₂, and —O(CH₂)nON[(CH₂)n CH₃)]₂, where n and m are from 1 to about 10; and/or a modification at the 5′ position: 5′-vinyl, 5′-methyl (R or S), a modification at the 4′ position, 4′-S, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and any combination thereof. In some embodiments, the unnatural nucleotide further comprises an unnatural backbone. In some embodiments, the unnatural backbone is selected from the group consisting of a phosphorothioate, chiral phosphorothioate, phosphorodithioate, phosphotriester, aminoalkylphosphotriester, C₁-C₁₀ phosphonates, 3′-alkylene phosphonate, chiral phosphonates, phosphinates, phosphoramidates, 3′-amino phosphoramidate, aminoalkylphosphoramidates, thionophosphoramidates, thionoalkylphosphonates, thionoalkylphosphotriesters, and boranophosphates. In some embodiments, the sgRNA has less than about 20%, 15%, 10%, 5%, 3%, 1%, or less off-target binding rate. In some embodiments, the nucleic acid molecule further comprises an additional nucleic acid molecule that encodes an additional single guide RNA (sgRNA) comprising a crRNA-tracrRNA scaffold. In some embodiments, the third nucleic acid molecule further comprises an additional unnatural nucleotide. In some embodiments, the first nucleic acid molecule, the second nucleic acid molecule, and the third nucleic acid molecule are encoded in one or more plasmids. In some embodiments, the incubating further comprises a transformation step. In some embodiments, the cell is a prokaryotic cell. In some embodiments, the cell is E. coli. In some embodiments, the cell is a fungal cell. In some embodiments, the cell is a yeast cell. In some embodiments, the cell is a eukaryotic cell. In some embodiments, the cell generates a stable cell line. In some embodiments, is a nucleic acid molecule containing an unnatural nucleotide produced by a process comprising incubating a cell with: (a) a first nucleic acid molecule encoding a Cas9 polypeptide or variants thereof; (b) a second nucleic acid molecule encoding two or more single guide RNAs (sgRNAs) wherein each sgRNA comprises a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and the two or more sgRNAs modulates replication of the modified third nucleic acid molecule leading to production of the nucleic acid molecule containing an unnatural nucleotide.

Disclosed herein, in certain embodiments, is a semi-synthetic organism produced by a process comprising incubating an organism with: (a) a first nucleic acid molecule encoding a Cas9 polypeptide or variants thereof; (b) a second nucleic acid molecule encoding a single guide RNAs (sgRNAs) wherein the sgRNA comprises a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and the sgRNA modulates replication of the modified third nucleic acid molecule leading to production of the semi-synthetic organism containing a nucleic acid molecule comprising an unnatural nucleotide. In some embodiments, the combination of Cas9 polypeptide or variants thereof and sgRNA decreases the replication rate of the modified third nucleic acid molecule by about 80%, 85%, 95%, 99%, or higher. In some embodiments, the modification is a substitution. In some embodiments, the modification is a deletion. In some embodiments, the modification is an insertion. In some embodiments, the organism further comprises an additional nucleic acid molecule that encodes an additional single guide RNA (sgRNA) comprising a crRNA-tracrRNA scaffold. In some embodiments, the organism is a cell. In some embodiments, the cell is a bacterial cell. In some embodiments, the cell is a fungal cell. In some embodiments, the cell is a yeast cell. In some embodiments, the cell is a eukaryotic cell. In some embodiments, the cell is a unicellular protozoan. In some embodiments, the cell generates a stable cell line.

Disclosed herein, in certain embodiments, is an isolated and purified plasmid comprising a sequence selected from SEQ ID NOs: 1-4. In some embodiments, the isolated and purified plasmid comprises a sequence of SEQ ID NO: 4. In some embodiments, the W motif of SEQ ID NO: 4 comprises a sequence selected from SEQ ID NOs: 5, 7, 9, 11, 13, 15, 17, 19, 21, 23, 25, or 27. In some embodiments, the Y motif of SEQ ID NO: 4 comprises a sequence selected from SEQ ID NOs: 6, 8, 10, 12, 14, 16, 18, 20, 22, 24, or 26.

Disclosed herein, in certain embodiments, is a kit comprising an isolated and purified plasmid of described above, and a nucleic acid molecule comprising an unnatural nucleotide.

Also described herein, in certain embodiments, is a kit comprising a stable cell line generated from a cell described above.

### BRIEF DESCRIPTION OF THE DRAWINGS

Various aspects of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C illustrate the relative cleavage efficiency (RCE) of variations of an sgRNA target against a DNA template. FIGS. 1A and 1B illustrate RCE given variations of a nucleotide, include using UBPs, at two different positions relative to a protospacer adjacent motif (PAM). FIGS. 1A and 1B disclose SEQ ID NOS 66-69, respectively, in order of appearance. FIG. 1C exemplifies a PAGE analysis to determine RCE of one of these variations. FIG. 1C discloses SEQ ID NOS 70 and 67, respectively, in order of appearance.

FIG. 2 exemplifies the pCas9/TK1-A plasmid.

FIG. 3 exemplifies the growth-regrowth cycle of the transformed E. coli first grown in the presence of the unnatural triphosphates to saturation, diluted 250-fold, and then grown to saturation again.

FIGS. 4A-4C illustrate percent UBP retention upon using different sgRNAs. FIG. 4A illustrates the percent of UBP retention when various types of guide RNA are used. FIG. 4B illustrates the sequences of both the target strand and the various sgRNA used. Target sequence and guide RNA sequences also included. FIG. 4B discloses SEQ ID NOS 71-74 and 74-75, respectively, in order of appearance. FIG. 4C exemplifies an analysis of UBP retention using the aforementioned sgRNAs.

FIGS. 5A-5B exemplify the major and minor mutations commonly observed in the target DNA. FIG. 5A illustrates the major mutation (dNaM→dT), and FIG. 5B illustrates the minor mutations (G, frameshift). FIGS. 5A and 5B disclose SEQ ID NOS 53-54 and 53-54, respectively, in order of appearance.

FIG. 6 illustrates the percentage of dNaM-dTPT3 retention, in either the coding or noncoding strand, at three different positions relative to the same PAM within the hGFP gene (6 sequences total). FIG. 6 discloses SEQ ID NOS 76-82, 77, 83, 79, 84, and 81, respectively, in order of appearance.

FIG. 7 illustrates the 16 sequences examined in which the dNaM of a dNaM-dTPT3 UBP was flanked by all possible nucleotides. FIG. 7 discloses SEQ ID NOS 85-100, respectively, in order of appearance.

### DETAILED DESCRIPTION OF THE INVENTION

The development of an unnatural base pair (UBP) allowing cells to store and retrieve increased information has a profound effect in practical applications, including human health applications by facilitating the production of proteins containing unnatural amino acids for development as therapeutics. However, retention of the UBP within a population of cells is sequence-dependent and in some sequences, the UBP is not sufficiently maintained or maintained at a reduced level, for practical applications (e.g. protein expression). In some instances, mutations within the sequences at the position of the unnatural base are introduced during the replication process, resulting in reduced retention of UBP within a population of cells.

Disclosed herein, in certain embodiments, are methods, compositions, cells, engineered microorganisms, plasmids, and kits for increased production of a nucleic acid molecule that comprises an unnatural nucleotide. In some instances, disclosed herein is an engineered cell comprising: (a) a first nucleic acid molecule encoding a Cas9 polypeptide or variants thereof; (b) a second nucleic acid molecule encoding a single guide RNA (sgRNA) comprising a crRNA-tracrRNA scaffold; and (c) a third nucleic acid molecule comprising an unnatural nucleotide; wherein the first nucleic acid molecule, the second nucleic acid molecule, and the third nucleic acid molecule are encoded in one or more plasmids, and the sgRNA encoded by the second nucleic acid molecule comprises a target motif that recognizes a modification at the unnatural nucleotide position within the third nucleic acid molecule.

In some embodiments, also provided herein include an in vivo method of increasing the production of a nucleic acid molecule containing an unnatural nucleotide, comprising incubating a cell with: (a) a first nucleic acid molecule encoding a Cas9 polypeptide or variants thereof, (b) a second nucleic acid molecule encoding a single guide RNA (sgRNA) comprising a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and sgRNA modulates replication of the modified third nucleic acid molecule to increase the production of the nucleic acid molecule containing an unnatural nucleotide.

In some embodiments, further provided herein include a nucleic acid molecule containing an unnatural nucleotide produced by a process comprising incubating a cell with: (a) a first nucleic acid molecule encoding a Cas9 polypeptide or variants thereof; (b) a second nucleic acid molecule encoding a single guide RNA (sgRNA) comprising a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and sgRNA modulates replication of the modified third nucleic acid molecule leading to production of the nucleic acid molecule containing an unnatural nucleotide.

In some embodiments, additional provided herein include a semi-synthetic organism produced by a process comprising incubating an organism with: (a) a first nucleic acid molecule encoding a Cas9 polypeptide or variants thereof, (b) a second nucleic acid molecule encoding a single guide RNAs (sgRNAs) wherein the sgRNA comprises a crRNA-tracrRNA scaffold; and (c) a third nucleic acid molecule comprising an unnatural nucleotide; wherein a modification at the unnatural nucleotide position within the third nucleic acid molecule generates a modified third nucleic acid molecule, and the combination of the Cas9 polypeptide or variants thereof and the sgRNA modulates replication of the modified third nucleic acid molecule leading to production of the semi-synthetic organism containing a nucleic acid molecule comprising an unnatural nucleotide.

In some embodiments, also described herein include an isolated and purified plasmid comprising a sequence selected from SEQ ID NOs: 1-4, and kits comprising one or more of the plasmids and/or stable cell lines described herein.

### CRISPR/CRISPR-Associated (Cas) Editing System

In some embodiments, methods, cells, and engineered microorganisms disclosed herein utilize a CRISPR/CRISPR-associated (Cas) system for modification of a nucleic acid molecule comprising an unnatural nucleotide. In some instances, the CRISPR/Cas system modulates retention of a modified nucleic acid molecule that comprises a modification at its unnatural nucleotide position. In some instances, the retention is a decrease in replication of the modified nucleic acid molecule. In some instances, the CRISPR/Cas system generates a double-stranded break within a modified nucleic acid molecule leading to degradation involving DNA repair proteins such as RecBCD and its associated nucleases.

In some embodiments, the CRISPR/Cas system involves (1) an integration of short regions of genetic material that are homologous to a nucleic acid molecule of interest comprising an unnatural nucleotide, called “spacers”, in clustered arrays in the host genome, (2) expression of short guiding RNAs (crRNAs) from the spacers, (3) binding of the crRNAs to specific portions of the nucleic acid molecule of interest referred to as protospacers, and (4) degradation of protospacers by CRISPR-associated nucleases (Cas). In some cases, a Type-II CRISPR system has been described in the bacterium Streptococcus pyogenes, in which Cas9 and two non-coding small RNAs (pre-crRNA and tracrRNA (trans-activating CRISPR RNA)) act in concert to target and degrade a nucleic acid molecule of interest in a sequence-specific manner (Jinek et al., “A Programmable Dual-RNA-Guided DNA Endonuclease in Adaptive Bacterial Immunity,” Science 337(6096):816-821 (August 2012, epub Jun. 28, 2012)).

In some instances, the two noncoding RNAs are further fused into one single guide RNA (sgRNA). In some instances, the sgRNA comprises a target motif that recognizes a modification at the unnatural nucleotide position within a nucleic acid molecule of interest. In some embodiments, the modification is a substitution, insertion, or deletion. In some cases, the sgRNA comprises a target motif that recognizes a substitution at the unnatural nucleotide position within a nucleic acid molecule of interest. In some cases, the sgRNA comprises a target motif that recognizes a deletion at the unnatural nucleotide position within a nucleic acid molecule of interest. In some cases, the sgRNA comprises a target motif that recognizes an insertion at the unnatural nucleotide position within a nucleic acid molecule of interest.

In some cases, the target motif is between 10 to 30 nucleotides in length. In some instances, the target motif is between 15 to 30 nucleotides in length. In some cases, the target motif is about 11, 12, 13, 14, 15, 16, 17, 18, 19, 20, 21, 22, 23, 24, 25, 26, 27, 28, 29, or 30 nucleotides in length. In some cases, the target motif is about 15, 16, 17, 18, 19, 20, 21, or 22 nucleotides in length.

In some cases, the sgRNA further comprises a protospacer adjacent motif (PAM) recognition element. In some instances, PAM is located adjacent to the 3′ terminus of the target motif. In some cases, a nucleotide within the target motif that forms Watson-Crick base pairing with the modification at the unnatural nucleotide position within the nucleic acid molecule of interest is located between 3 to 22, between 5 to 20, between 5 to 18, between 5 to 15, between 5 to 12, or between 5 to 10 nucleotides from the 5′ terminus of PAM. In some cases, a nucleotide within the target motif that forms Watson-Crick base pairing with the modification at the unnatural nucleotide position within the nucleic acid molecule of interest is located about 3, 4, 5, 6, 7, 8, 9, 10, 11, 12, 13, 14, or 15 nucleotides from the 5′ terminus of PAM.

In some instances, a CRISPR/Cas system utilizes a Cas9 polypeptide or a variant thereof. Cas9 is a double stranded nuclease with two active cutting sites, one for each strand of the double helix. In some instances, the Cas9 polypeptide or variants thereof generate a double-stranded break. In some cases, the Cas9 polypeptide is a wild-type Cas9. In some instances, the Cas9 polypeptide is an optimized Cas9 for expression in a cell and/or engineered microorganism described herein.

In some embodiments, the Cas9/sgRNA complex binds to a portion of the nucleic acid molecule of interest (e.g., DNA) that contains a sequence match to, for example, the 17-20 nucleotides of the sgRNA upstream of PAM. Once bound, two independent nuclease domains in Cas9 then each cleaves one of the DNA strands 3 bases upstream of the PAM, leaving a blunt end DNA double stranded break (DSB). The presence of DSB then results, in some instances, to degradation of the DNA of interest by RecBCD and its associated nucleases.

In some instances, the Cas9/sgRNA complex modulates retention of a modified nucleic acid molecule that comprises a modification at its unnatural nucleotide position. In some instances, the retention is a decrease in replication of the modified nucleic acid molecule. In some cases, the Cas9/sgRNA decreases the replication rate of the modified nucleic acid molecule by about 80%, 85%, 95%, 99%, or higher.

In some instances, the production of the nucleic acid molecule comprising an unnatural nucleotide increases by about 30%, 40%, 50%, 55%, 60%, 65%, 70%, 75%, 80%, 85%, 90%, 95%, 96%, 97%, 98%, 99%, or higher. In some instances, the production of the nucleic acid molecule comprising an unnatural nucleotide increases by about 50%, 60%, 70%, 80%, 90%, 95%, 96%, 97%, 98%, 99%, or higher.

In some cases, the retention of the nucleic acid molecule comprising an unnatural nucleotide increases by about 30%, 40%, 50%, 55%, 60%, 65%, 70%, 75%, 80%, 85%, 90%, 95%, 96%, 97%, 98%, 99%, or higher. In some instances, the retention of the nucleic acid molecule comprising an unnatural nucleotide increases by about 50%, 60%, 70%, 80%, 90%, 95%, 96%, 97%, 98%, 99%, or higher.

In some embodiments, the CRISPR/Cas system comprises two or more sgRNAs. In some instances, each of the two or more sgRNAs independently comprises a target motif that recognizes a modification at the unnatural nucleotide position within a nucleic acid molecule of interest. In some embodiments, the modification is a substitution, insertion, or deletion. In some cases, each of the two or more sgRNAs comprises a target motif that recognizes a substitution at the unnatural nucleotide position within a nucleic acid molecule of interest. In some cases, each of the two or more sgRNAs comprises a target motif that recognizes a deletion at the unnatural nucleotide position within a nucleic acid molecule of interest. In some cases, each of the two or more sgRNAs comprises a target motif that recognizes an insertion at the unnatural nucleotide position within a nucleic acid molecule of interest.

In some embodiments, the specificity of binding of the CRISPR components to the nucleic acid molecule of interest is controlled by the non-repetitive spacer elements in the pre-crRNA portion of sgRNA, which upon transcription along with the tracrRNA portion, directs the Cas9 nuclease to the protospacer:crRNA heteroduplex and induces double-strand breakage (DSB) formation. In some instances, the specificity of sgRNA is about 80%, 85%, 90%, 95%, 96%, 97%, 98%, 99%, or higher. In some instances, sgRNA has less than about 20%, 15%, 10%, 5%, 3%, 1%, or less off-target binding rate.

### Nucleic Acid Molecules

In some embodiments, a nucleic acid (e.g., also referred to herein as nucleic acid molecule of interest) is from any source or composition, such as DNA, cDNA, gDNA (genomic DNA), RNA, siRNA (short inhibitory RNA), RNAi, tRNA, mRNA or rRNA (ribosomal RNA), for example, and is in any form (e.g., linear, circular, supercoiled, single-stranded, double-stranded, and the like). In some embodiments, nucleic acids comprise nucleotides, nucleosides, or polynucleotides. In some cases, nucleic acids comprise natural and unnatural nucleic acids. In some cases, a nucleic acid also comprises unnatural nucleic acids, such as DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Exemplary natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Exemplary natural deoxyribonucleotides include dATP, dTTP, dCTP, dGTP, dADP, dTDP, dCDP, dGDP, dAMP, dTMP, dCMP, and dGMP. Exemplary natural ribonucleotides include ATP, UTP, CTP, GTP, ADP, UDP, CDP, GDP, AMP, UMP, CMP, and GMP. For RNA, the uracil base is uridine. A nucleic acid sometimes is a vector, plasmid, phagemid, autonomously replicating sequence (ARS), centromere, artificial chromosome, yeast artificial chromosome (e.g., YAC) or other nucleic acid able to replicate or be replicated in a host cell. In some cases, an unnatural nucleic acid is a nucleic acid analogue. In additional cases, an unnatural nucleic acid is from an extracellular source. In other cases, an unnatural nucleic acid is available to the intracellular space of an organism provided herein, e.g., a genetically modified organism.

### Unnatural Nucleic Acids

A nucleotide analog, or unnatural nucleotide, comprises a nucleotide which contains some type of modification to either the base, sugar, or phosphate moieties. In some embodiments, a modification comprises a chemical modification. In some cases, modifications occur at the 3′OH or 5′OH group, at the backbone, at the sugar component, or at the nucleotide base. Modifications, in some instances, optionally include non-naturally occurring linker molecules and/or of interstrand or intrastrand cross links. In one aspect, the modified nucleic acid comprises modification of one or more of the 3′OH or 5′OH group, the backbone, the sugar component, or the nucleotide base, and/or addition of non-naturally occurring linker molecules.

In one aspect, a modified backbone comprises a backbone other than a phosphodiester backbone. In one aspect, a modified sugar comprises a sugar other than deoxyribose (in modified DNA) or other than ribose (modified RNA). In one aspect, a modified base comprises a base other than adenine, guanine, cytosine or thymine (in modified DNA) or a base other than adenine, guanine, cytosine or uracil (in modified RNA).

In some embodiments, the nucleic acid comprises at least one modified base. In some instances, the nucleic acid comprises 2, 3, 4, 5, 6, 7, 8, 9, 10, 15, 20, or more modified bases. In some cases, modifications to the base moiety include natural and synthetic modifications of A, C, G, and T/U as well as different purine or pyrimidine bases. In some embodiments, a modification is to a modified form of adenine, guanine cytosine or thymine (in modified DNA) or a modified form of adenine, guanine cytosine or uracil (modified RNA).

A modified base of a unnatural nucleic acid includes, but is not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C-CI¼) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9.807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crookeand Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993.

Unnatural nucleic acids comprising various heterocyclic bases and various sugar moieties (and sugar analogs) are available in the art, and the nucleic acid in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d]pyrimidin-5-yl, 2-amino-4-oxopyrolo [2, 3-d] pyrimidin-5-yl, 2-amino-4-oxopyrrolo [2.3-d]pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, a modified base of a unnatural nucleic acid is depicted below, wherein the wavy line identifies a point of attachment to the (deoxy)ribose or ribose.

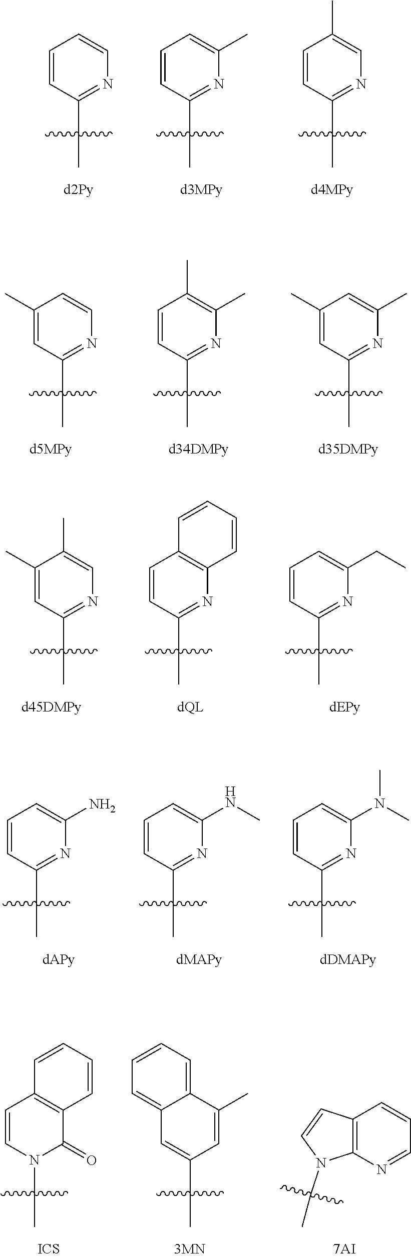

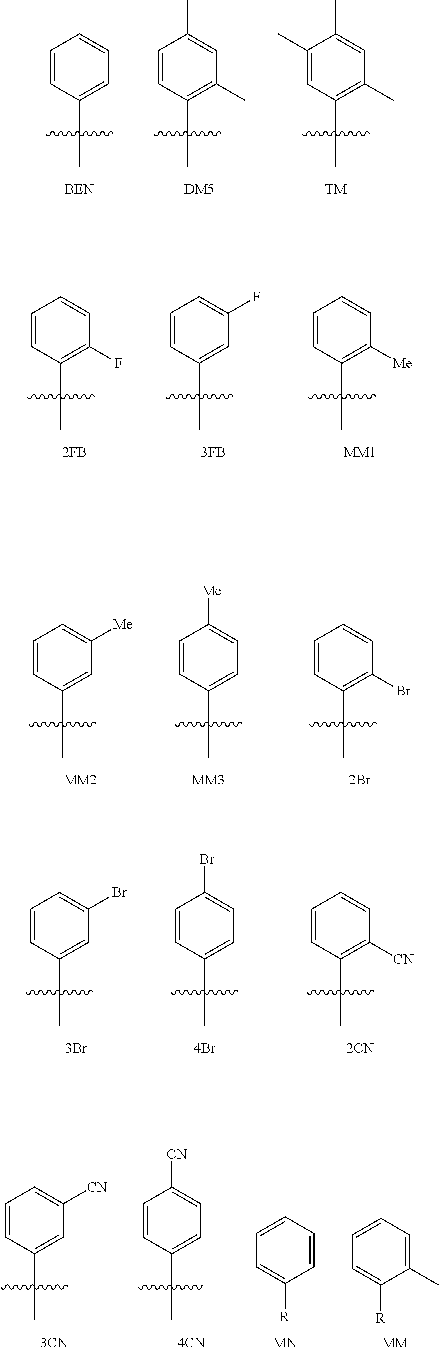

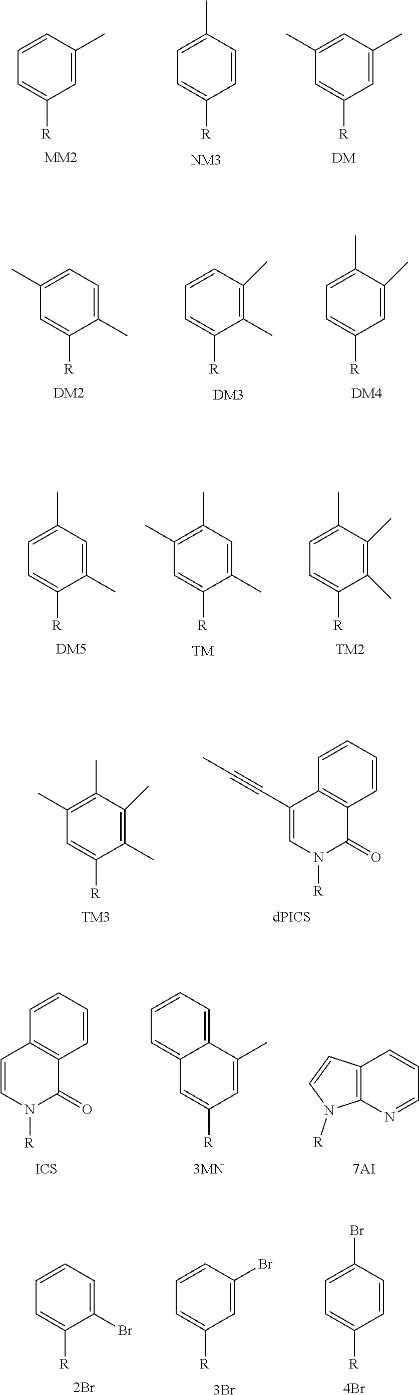

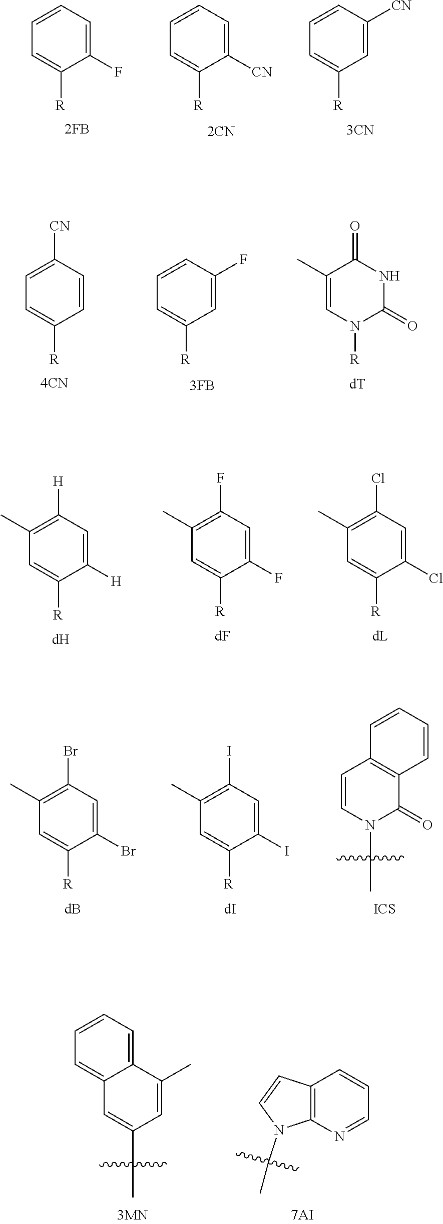

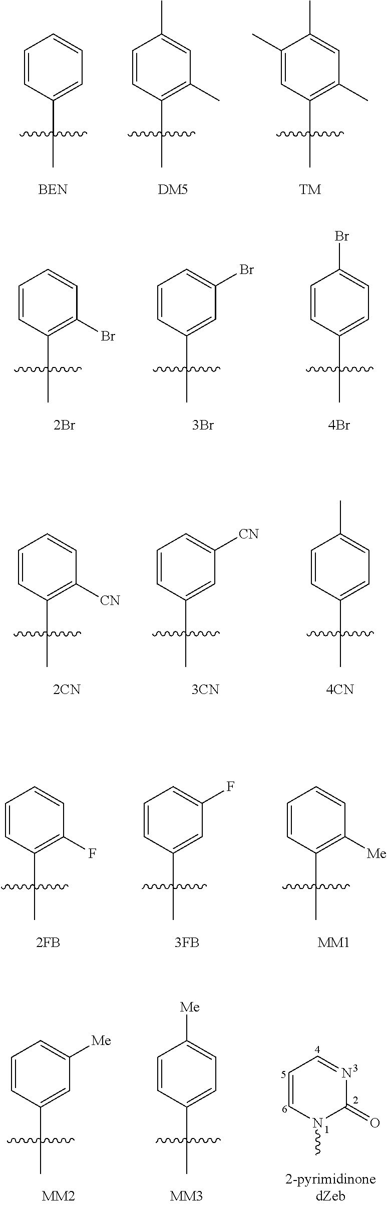

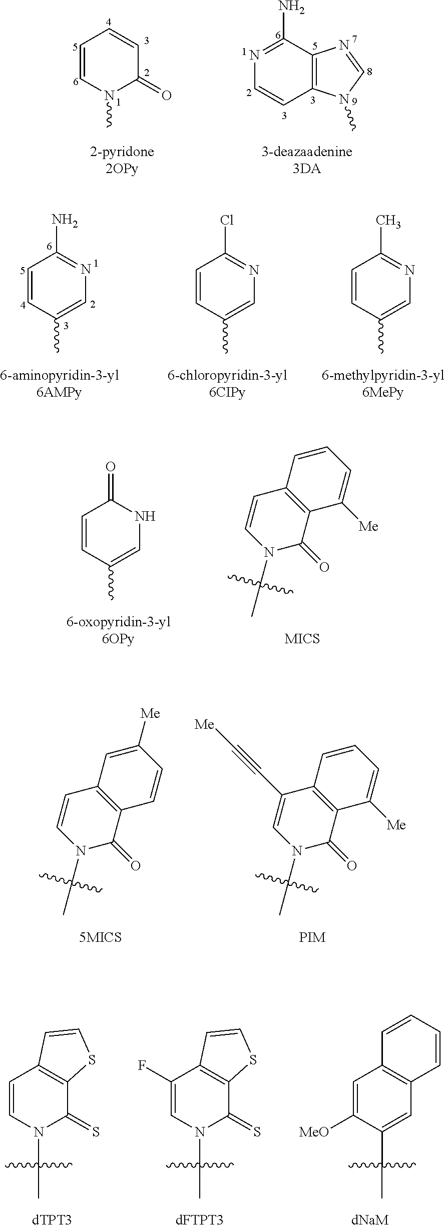

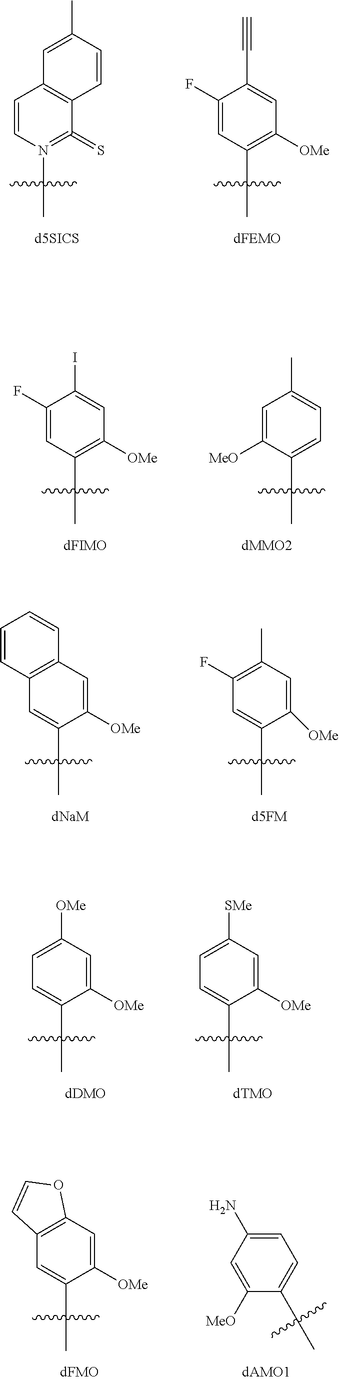

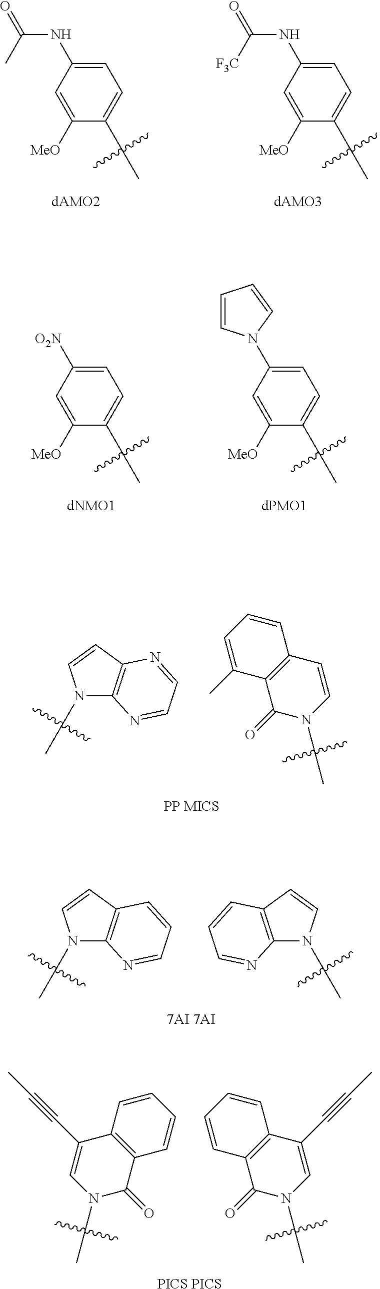

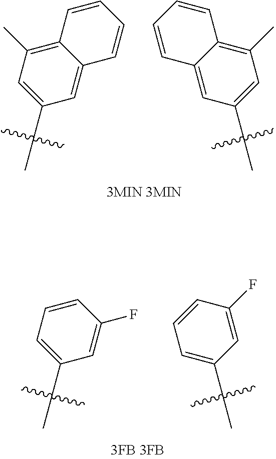

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotri ester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337).

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414).

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substitutent groups (including 5′ and/or 2′ substituent groups; bridging of two ring atoms to form bicyclic nucleic acids (BNA); replacement of the ribosyl ring oxygen atom with S, N(R), or C(Ri)(R₂) (R=H, C₁-C₁₂ alkyl or a protecting group); and combinations thereof. Examples of chemically modified sugars can be found in WO2008/101157, US2005/0130923, and WO2007/134181.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ONH₂, and —O(CH₂)˜ON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, NY; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM50J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Nucleic Acid Base Pairing Properties

In some embodiments, an unnatural nucleic acid forms a base pair with another nucleic acid. In some embodiments, a stably integrated unnatural nucleic acid is an unnatural nucleic acid that can form a base pair with another nucleic acid, e.g., a natural or unnatural nucleic acid. In some embodiments, a stably integrated unnatural nucleic acid is an unnatural nucleic acid that can form a base pair with another unnatural nucleic acid (unnatural nucleic acid base pair (UBP)). For example, a first unnatural nucleic acid can form a base pair with a second unnatural nucleic acid. For example, one pair of unnatural nucleotide triphosphates that can base pair when incorporated into nucleic acids include a triphosphate of d5SICS (d5SICSTP) and a triphosphate of dNaM (dNaMTP). Such unnatural nucleotides can have a ribose or deoxyribose sugar moiety. In some embodiments, an unnatural nucleic acid does not substantially form a base pair with a natural nucleic acid (A, T, G, C). In some embodiments, a stably integrated unnatural nucleic acid can form a base pair with a natural nucleic acid.

In some embodiments, a stably integrated unnatural nucleic acid is an unnatural nucleic acid that can form a UBP, but does not substantially form a base pair with each of the four natural nucleic acids. In some embodiments, a stably integrated unnatural nucleic acid is an unnatural nucleic acid that can form a UBP, but does not substantially form a base pair with one or more natural nucleic acids. For example, a stably integrated unnatural nucleic acid may not substantially form a base pair with A, T, and, C, but can form a base pair with G. For example, a stably integrated unnatural nucleic acid may not substantially form a base pair with A, T, and, G, but can form a base pair with C. For example, a stably integrated unnatural nucleic acid may not substantially form a base pair with C, G, and, A, but can form a base pair with T. For example, a stably integrated unnatural nucleic acid may not substantially form a base pair with C, G, and, T, but can form a base pair with A. For example, a stably integrated unnatural nucleic acid may not substantially form a base pair with A and T, but can form a base pair with C and G. For example, a stably integrated unnatural nucleic acid may not substantially form a base pair with A and C, but can form a base pair with T and G. For example, a stably integrated unnatural nucleic acid may not substantially form a base pair with A and G, but can form a base pair with C and T. For example, a stably integrated unnatural nucleic acid may not substantially form a base pair with C and T, but can form a base pair with A and G. For example, a stably integrated unnatural nucleic acid may not substantially form a base pair with C and G, but can form a base pair with T and G. For example, a stably integrated unnatural nucleic acid may not substantially form a base pair with T and G, but can form a base pair with A and G. For example, a stably integrated unnatural nucleic acid may not substantially form a base pair with, G, but can form a base pair with A, T, and, C. For example, a stably integrated unnatural nucleic acid may not substantially form a base pair with, A, but can form a base pair with G, T, and, C. For example, a stably integrated unnatural nucleic acid may not substantially form a base pair with, T, but can form a base pair with G, A, and, C. For example, a stably integrated unnatural nucleic acid may not substantially form a base pair with, C, but can form a base pair with G, T, and, A.

Exemplary, unnatural nucleotides capable of forming an unnatural DNA or RNA base pair (UBP) under conditions in vivo includes, but is not limited to, 5SICS, d5SICS, NAM, dNaM, and combinations thereof. In some embodiments, unnatural nucleotides include:

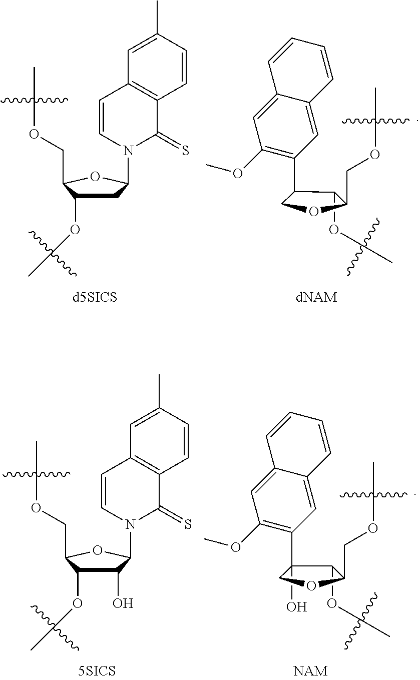

### Engineered Organism

In some embodiments, methods and plasmids disclosed herein is further used to generate engineered organism, e.g. an organism that incorporates and replicates an unnatural nucleotide or an unnatural nucleic acid base pair (UBP) with improved UBP retention and also transcribes and translates the nucleic acid containing the unnatural nucleotide or unnatural nucleic acid base pair into a protein containing an unnatural amino acid residue. In some instances, the organism is a semi-synthetic organism (SSO). In some instances, the SSO is a cell.

In some instances, the cell employed is genetically transformed with an expression cassette encoding a heterologous protein, e.g., a nucleotide triphosphate transporter capable of transporting unnatural nucleotide triphosphates into the cell, a CRISPR/Cas9 system to remove modifications at the unnatural nucleotide triphosphate positions, and/or a polymerase with high fidelity for an unnatural nucleic acid, so that the unnatural nucleotides are incorporated into cellular nucleic acids and e.g., form unnatural base pairs under in vivo conditions. In some instances, cells further comprise enhanced activity for unnatural nucleic acid uptake. In some cases, cells further comprise enhanced activity for unnatural nucleic acid import. In some cases, cells further comprise enhanced polymerase activity for unnatural nucleic acids.

In some embodiments, Cas9 and sgRNA are encoded on separate plasmids. In some instances, Cas9 and sgRNA are encoded on the same plasmid. In some cases, the nucleic acid molecule encoding Cas9, sgRNA, or a nucleic acid molecule comprising an unnatural nucleotide are located on one or more plasmids. In some instances, Cas9 is encoded on a first plasmid and the sgRNA and the nucleic acid molecule comprising an unnatural nucleotide are encoded on a second plasmid. In some instances, Cas9, sgRNA, and the nucleic acid molecule comprising an unnatural nucleotide are encoded on the same plasmid. In some instances, the nucleic acid molecule comprises two or more unnatural nucleotides.

In some instances, a first plasmid encoding Cas9 and sgRNA and a second plasmid encoding a nucleic acid molecule comprising an unnatural nucleotide are introduced into an engineered microorganism. In some instances, a first plasmid encoding Cas9 and a second plasmid encoding sgRNA and a nucleic acid molecule comprising an unnatural nucleotide are introduced into an engineered microorganism. In some instances, a plasmid encoding Cas9, sgRNA and a nucleic acid molecule comprising an unnatural nucleotide is introduced into an engineered microorganism. In some instances, the nucleic acid molecule comprises two or more unnatural nucleotides.

In some embodiments, a living cell is generated that incorporates within its nucleic acids at least one unnatural nucleotide and/or at least one unnatural base pair (UBP). In some instances, the unnatural base pair includes a pair of unnatural mutually base-pairing nucleotides capable of forming the unnatural base pair under in vivo conditions, when the unnatural mutually base-pairing nucleotides, as their respective triphosphates, are taken up into the cell by action of a nucleotide triphosphate transporter. The cell can be genetically transformed by an expression cassette encoding a nucleotide triphosphate transporter so that the nucleotide triphosphate transporter is expressed and is available to transport the unnatural nucleotides into the cell. The cell can be genetically transformed by an expression cassette encoding a polymerase so that the polymerase is expressed and is available to incorporate unnatural nucleotides into the cell's nucleic acids. The cell can be a prokaryotic or eukaryotic cell, and the pair of unnatural mutually base-pairing nucleotides, as their respective triphosphates, can be a triphosphate of d5SICS (d5SICSTP) and a triphosphate of dNaM (dNaMTP).

In some embodiments, cells are genetically transformed cells with a nucleic acid, e.g., an expression cassette encoding a nucleotide triphosphate transporter capable of transporting such unnatural nucleotides into the cell. A cell can comprise a heterologous nucleotide triphosphate transporter, where the heterologous nucleotide triphosphate transporter can transport natural and unnatural nucleotide triphosphates into the cell. A cell can comprise a heterologous polymerase, where the heterologous polymerase has activity for an unnatural nucleic acid.

In some cases, a method described herein also include contacting a genetically transformed cell with the respective triphosphate forms unnatural nucleotides, in the presence of potassium phosphate and/or an inhibitor of phosphatases or nucleotidases. During or after such contact, the cell can be placed within a life-supporting medium suitable for growth and replication of the cell. The cell can be maintained in the life-supporting medium so that the respective triphosphate forms of unnatural nucleotides are incorporated into nucleic acids within the cells, and through at least one replication cycle of the cell. The pair of unnatural mutually base-pairing nucleotides as a respective triphosphate, can comprise a triphosphate of d5SICS (d5SICSTP) and a triphosphate of dNaM (dNaMTP), the cell can be E. coli, and the d5SICSTP and dNaMTP can be efficiently imported into E. coli by the transporter PtNTT2, wherein an E. coli polymerase, such as Pol I, can efficiently use the unnatural triphosphates to replicate DNA, thereby incorporating unnatural nucleotides and/or unnatural base pairs into cellular nucleic acids within the cellular environment.

By practice of a method of the invention, the person of ordinary skill can obtain a population of a living and propagating cells that has at least one unnatural nucleotide and/or at least one unnatural base pair (UBP) within at least one nucleic acid maintained within at least some of the individual cells, wherein the at least one nucleic acid is stably propagated within the cell, and wherein the cell expresses a nucleotide triphosphate transporter suitable for providing cellular uptake of triphosphate forms of one or more unnatural nucleotides when contacted with (e.g., grown in the presence of) the unnatural nucleotide(s) in a life-supporting medium suitable for growth and replication of the organism.

After transport into the cell by the nucleotide triphosphate transporter, the unnatural base-pairing nucleotides are incorporated into nucleic acids within the cell by cellular machinery, e.g., the cell's own DNA and/or RNA polymerases, a heterologous polymerase, or a polymerase that has been evolved using directed evolution (Chen T, Romesberg F E, FEBS Lett. 2014 Jan. 21; 588(2):219-29; Betz K et al., J Am Chem Soc. 2013 Dec. 11; 135(49):18637-43). The unnatural nucleotides can be incorporated into cellular nucleic acids such as genomic DNA, genomic RNA, mRNA, structural RNA, microRNA, and autonomously replicating nucleic acids (e.g., plasmids, viruses, or vectors).

In some cases, genetically engineered cells are generated by introduction of nucleic acids, e.g., heterologous nucleic acids, into cells. Any cell described herein can be a host cell and can comprise an expression vector. In one embodiment, the host cell is a prokaryotic cell. In another embodiment, the host cell is E. coli. In some embodiments, a cell comprises one or more heterologous polynucleotides. Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, particle bombardment and the like. In some instances the addition of carrier molecules (e.g., bis-benzimdazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some instances, genetic transformation is obtained using direct transfer of an expression cassette, in but not limited to, plasmids, viral vectors, viral nucleic acids, phage nucleic acids, phages, cosmids, and artificial chromosomes, or via transfer of genetic material in cells or carriers such as cationic liposomes. Such methods are available in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

For example, a nucleotide triphosphate transporter or polymerase nucleic acid molecule, expression cassette and/or vector can be introduced to a cell by any method including, but not limited to, calcium-mediated transformation, electroporation, microinjection, lipofection, particle bombardment and the like.

In some cases, a cell comprises unnatural nucleotide triphosphates incorporated into one or more nucleic acids within the cell. For example, the cell can be a living cell capable of incorporating at least one unnatural nucleotide within DNA or RNA maintained within the cell. The cell can also incorporate at least one unnatural base pair (UBP) comprising a pair of unnatural mutually base-pairing nucleotides into nucleic acids within the cell under in vivo conditions, wherein the unnatural mutually base-pairing nucleotides, e.g., their respective triphosphates, are taken up into the cell by action of a nucleotide triphosphate transporter, the gene for which is present (e.g., was introduced) into the cell by genetic transformation. For example, upon incorporation into the nucleic acid maintained within s cell, d5SICS and dNaM can form a stable unnatural base pair that can be stably propagated by the DNA replication machinery of an organism, e.g., when grown in a life-supporting medium comprising d5SICS and dNaM.

In some cases, cells are capable of replicating an unnatural nucleic acid. Such methods can include genetically transforming the cell with an expression cassette encoding a nucleotide triphosphate transporter capable of transporting into the cell, as a respective triphosphate, one or more unnatural nucleotides under in vivo conditions. Alternatively, a cell can be employed that has previously been genetically transformed with an expression cassette that can express an encoded nucleotide triphosphate transporter. The method can also include contacting or exposing the genetically transformed cell to potassium phosphate and the respective triphosphate forms of at least one unnatural nucleotide (for example, two mutually base-pairing nucleotides capable of forming the unnatural base pair (UBP)) in a life-supporting medium suitable for growth and replication of the cell, and maintaining the transformed cell in the life-supporting medium in the presence of the respective triphosphate forms of at least one unnatural nucleotide (for example, two mutually base-pairing nucleotides capable of forming the unnatural base pair (UBP)) under in vivo conditions, through at least one replication cycle of the cell.

In some embodiments, a cell comprises a stably incorporated unnatural nucleic acid. Some embodiments comprise a cell (e.g., as E. coli) that stably incorporates nucleotides other than A, G, T, and C within nucleic acids maintained within the cell. For example, the nucleotides other than A, G, T, and C can be d5SICS and dNaM, which upon incorporation into nucleic acids of the cell, can form a stable unnatural base pair within the nucleic acids. In one aspect, unnatural nucleotides and unnatural base pairs can be stably propagated by the replication apparatus of the organism, when an organism transformed with the gene for the triphosphate transporter, is grown in a life-supporting medium that includes potassium phosphate and the triphosphate forms of d5SICS and dNaM.

In some cases, a cell comprises an expanded genetic alphabet. A cell can comprise a stably incorporated unnatural nucleic acid. In some embodiments, a cell with an expanded genetic alphabet comprises an unnatural nucleic acid that can form a base pair (bp) with another nucleic acid, e.g., a natural or unnatural nucleic acid. In some embodiments, a cell with an expanded genetic alphabet comprises an unnatural nucleic acid that is hydrogen bonded to another nucleic acid. In some embodiments, a cell with an expanded genetic alphabet comprises an unnatural nucleic acid that is not hydrogen bonded to another nucleic acid to which it is base paired. In some embodiments, a cell with an expanded genetic alphabet comprises an unnatural nucleic acid that base pairs to another nucleic acid via hydrophobic interactions. In some embodiments, a cell with an expanded genetic alphabet comprises an unnatural nucleic acid that base pairs to another nucleic acid via non-hydrogen bonding interactions. A cell with an expanded genetic alphabet can be a cell that can copy a homologous nucleic acid to form a nucleic acid comprising an unnatural nucleic acid. A cell with an expanded genetic alphabet can be a cell comprising an unnatural nucleic acid base paired with another unnatural nucleic acid (unnatural nucleic acid base pair (UBP)).

In some embodiments, cells form unnatural DNA base pairs (UBPs) from the imported unnatural nucleotides under in vivo conditions. In some embodiments potassium phosphate and/or inhibitors of phosphatase and/or nucleotidase activities can facilitate transport of unnatural nucleic acids. The methods include use of a cell that expresses a heterologous nucleotide triphosphate transporter. When such a cell is contacted with one or more nucleotide triphosphates, the nucleotide triphosphates are transported into the cell. The cell can be in the presence of potassium phosphate and/or inhibitors of phosphatase and nucleotidase. Unnatural nucleotide triphosphates can be incorporated into nucleic acids within the cell by the cell's natural machinery and, for example, can mutually base-pair to form unnatural base pairs within the nucleic acids of the cell.

In some embodiments, a UBP can be incorporated into a cell or population of cells when exposed to unnatural triphosphates. In some embodiments a UBP can be incorporated into a cell or population of cells when substantially consistently exposed to unnatural triphosphates. In some embodiments, replication of a UBP does not result in a substantially reduced growth rate. In some embodiments, replication expression of a heterologous protein, e.g., a nucleotide triphosphate transport does not result in a substantially reduced growth rate.

In some embodiments, induction of expression of a heterologous gene, e.g., an NTT, in a cell can result in slower cell growth and increased unnatural nucleic acid uptake compared to the growth and uptake of a cell without induction of expression of the heterologous gene. In some embodiments, induction of expression of a heterologous gene, e.g., an NTT, in a cell can result in increased cell growth and increased unnatural nucleic acid uptake compared to the growth and uptake of a cell without induction of expression of the heterologous gene.

In some embodiments, a UBP is incorporated during a log growth phase. In some embodiments, a UBP is incorporated during a non-log growth phase. In some embodiments, a UBP is incorporated during a substantially linear growth phase. In some embodiments a UBP is stably incorporated into a cell or population of cells after growth for a time period. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 50 or more duplications. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, or 24 hours of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days of growth. For example, a UBP can be stably incorporated into a cell or population of cells after growth for at least about 1, 2, 3, 4, 5, 6, 7, 8, 9, 10, 11, or 12 months of growth. For example, a UBP can be stably incorporated into a cell or population of cells after growth for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50 years of growth.

In some embodiments, a cell further utilizes a polymerase described herein to generate a mutant mRNA which contains a mutant codon that comprises one or more unnatural nucleic acid base. In some instances, a cell further utilizes a polymerase disclosed herein to generate a mutant tRNA which contains a mutant anticodon that comprises one or more unnatural nucleic acid base. In some instances, the mutant anticodon represents an unnatural amino acid. In some instances, the anticodon of the mutant tRNA pairs with the codon of the mutant mRNA during translation to synthesis a protein that contains an unnatural amino acid.

As used herein, an amino acid residue can refer to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” can refer to a molecule containing both an amino group and a carboxyl group bound to a carbon which is designated the α-carbon.

The term “β-amino acid” can refer to a molecule containing both an amino group and a carboxyl group in a β configuration.

“Naturally occurring amino acid” can refer to any one of the twenty amino acids commonly found in peptides synthesized in nature, and known by the one letter abbreviations A, R, N, C, D, Q, E, G, H, I, L, K, M, F, P, S, T, W, Y and V.

The following table shows a summary of the properties of natural amino acids:

| | 3- | 1- | Side- | Side-chain | |
|---|---|---|---|---|---|
| | Letter | Letter | chain | charge | Hydropathy |
| Amino Acid | Code | Code | Polarity | (pH 7.4) | Index |
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | polar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| | | | | (10%) | |
| | | | | neutral | |
| | | | | (90%) | |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

“Hydrophobic amino acids” include small hydrophobic amino acids and large hydrophobic amino acids. “Small hydrophobic amino acid” can be glycine, alanine, proline, and analogs thereof. “Large hydrophobic amino acids” can be valine, leucine, isoleucine, phenylalanine, methionine, tryptophan, and analogs thereof. “Polar amino acids” can be serine, threonine, asparagine, glutamine, cysteine, tyrosine, and analogs thereof. “Charged amino acids” can be lysine, arginine, histidine, aspartate, glutamate, and analogs thereof.

An “amino acid analog” can be a molecule which is structurally similar to an amino acid and which can be substituted for an amino acid in the formation of a peptidomimetic macrocycle Amino acid analogs include, without limitation, j-amino acids and amino acids where the amino or carboxy group is substituted by a similarly reactive group (e.g., substitution of the primary amine with a secondary or tertiary amine, or substitution of the carboxy group with an ester).

A “non-natural amino acid” can be an amino acid which is not one of the twenty amino acids commonly found in peptides synthesized in nature, and known by the one letter abbreviations A, R, N, C, D, Q, E, G, H, I, L, K, M, F, P, S, T, W, Y and V.

Amino acid analogs can include 3-amino acid analogs. Examples of 3-amino acid analogs include, but are not limited to, the following: cyclic 3-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(3-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino-4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro-butyric acid; 3-aminoadipic acid; D-β-phenylalanine; β-leucine; L-β-homoalanine; L-β-homoaspartic acid γ-benzyl ester; L-β-homoglutamic acid 6-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-3-homolysine; Nω-L-β-homoarginine; O-benzyl-L-β-homohydroxyproline; O-benzyl-L-β-homoserine; O-benzyl-L-β-homothreonine; O-benzyl-L-β-homotyrosine; γ-trityl-L-β-homoasparagine; (R)-β-phenylalanine; L-β-homoaspartic acid γ-t-butyl ester; L-β-homoglutamic acid δ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L-β-homoserine; O-t-butyl-L-β-homothreonine; O-t-butyl-L-β-homotyrosine; 2-aminocyclopentane carboxylic acid; and 2-aminocyclohexane carboxylic acid.

Amino acid analogs can include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; β-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanin; β-cyclohexyl-D-alanine; β-cyclohexyl-L-alanine; β-cyclopenten-1-yl-alanine; β-cyclopentyl-alanine; β-cyclopropyl-L-Ala-OH.dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH.dicyclohexylammonium salt; 4-fluoro-D-phenylglycine; 4-fluoro-L-phenylglycine; 4-hydroxy-D-phenylglycine; 5,5,5-trifluoro-leucine; 6-aminohexanoic acid; cyclopentyl-D-Gly-OH.dicyclohexylammonium salt; cyclopentyl-Gly-OH.dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; (2-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine.dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-3-(2,4-dinitrophenyl))-L-α,β-diaminopropionic acid; (N-3-1-(4,4-dimethyl-2,6-dioxocyclohex-1-ylidene)ethyl)-D-α,β-diaminopropionic acid; (N-3-1-(4,4-dimethyl-2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex-1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

Amino acid analogs can include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-ornithine; Nω-nitro-D-arginine; Nω-nitro-L-arginine; α-methyl-ornithine; 2,6-diaminoheptanedioic acid; L-ornithine; (Nδ-1-(4,4-dimethyl-2,6-dioxo-cyclohex-1-ylidene)ethyl)-D-ornithine; (Nδ-1-(4,4-dimethyl-2,6-dioxo-cyclohex-1-ylidene)ethyl)-L-ornithine; (Nδ-4-methyltrityl)-D-ornithine; (Nδ-4-methyltrityl)-L-ornithine; D-ornithine; L-ornithine; Arg(Me)(Pbf)-OH; Arg(Me)₂-OH (asymmetrical); Arg(Me)2-OH (symmetrical); Lys(ivDde)-OH; Lys(Me)2-OH·HCl; Lys(Me3)-OH chloride; Nω-nitro-D-arginine; and Nω-nitro-L-arginine.

Amino acid analogs can include analogs of aspartic or glutamic acids. Examples of amino acid analogs of aspartic and glutamic acids include, but are not limited to, the following: α-methyl-D-aspartic acid; α-methyl-glutamic acid; α-methyl-L-aspartic acid; γ-methylene-glutamic acid; (N-γ-ethyl)-L-glutamine; [N-α-(4-aminobenzoyl)]-L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy-L-glutamic acid 7,7-di-t-butyl ester; Glu(OAll)-OH; L-Asu(OtBu)-OH; and pyroglutamic acid.

Amino acid analogs can include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Amino acid analogs can include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Amino acid analogs can include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

Amino acid analogs can include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Amino acid analogs can include analogs of tryptophan. Examples of amino acid analogs of tryptophan include, but are not limited to, the following: α-methyl-tryptophan; j-(3-benzothienyl)-D-alanine; β-(3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl-tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

Amino acid analogs can be racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a 3-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some embodiments, an unnatural amino acid is an unnatural amino acid described in Liu C. C., Schultz, P. G. Annu. Rev. Biochem. 2010, 79, 413.

### Cell Types

In some embodiments, many types of cells/microorganisms are used, e.g., for transforming or genetically engineering. In some embodiments, a cell is a prokaryotic or eukaryotic cell. In some cases, the cell is a microorganism such as a bacterial cell, fungal cell, yeast, or unicellular protozoan. In other cases, the cell is a eukaryotic cell, such as a cultured animal, plant, or human cell. In additional cases, the cell is present in an organism such as a plant or animal.

In some embodiments, an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba). In some embodiments, an engineered microorganism is a fungus. In some embodiments, an engineered organism is a yeast.

Any suitable yeast may be selected as a host microorganism, engineered microorganism, genetically modified organism or source for a heterologous or modified polynucleotide. Yeast include, but are not limited to, Yarrowia yeast (e.g., Y. lipolytica (formerly classified as Candida lipolytica)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a suitable yeast is of the genus Arachniotus, Aspergillus, Aureobasidium, Auxarthron, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pachysolen, Penicillium, Pichia, Rhodosporidium, Rhodotorula, Rhodotorula, Saccharomyces, Schizosaccharomyces, Scopulariopsis, Sepedonium, Trichosporon, or Yarrowia. In some embodiments, a suitable yeast is of the species Arachniotus flavoluteus, Aspergillus flavus, Aspergillus fumigatus, Aspergillus niger, Aureobasidium pullulans, Auxarthron thaxteri, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporuim keratinophilum, Coccidiodes immitis, Cryptococcus albidus var. diffluens, Cryptococcus laurentii, Cryptococcus neofomans, Debaryomyces hansenii, Gymnoascus dugwayensis, Hansenula anomala, Histoplasma capsulatum, Issatchenkia occidentalis, Isstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutaneum, Trichosporon pullans, Yarrowia lipolytica, or Yarrowia lipolytica (formerly classified as Candida lipolytica). In some embodiments, a yeast is a Y. lipolytica strain that includes, but is not limited to, ATCC20362, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a Candida species (i.e., Candida spp.) yeast. Any suitable Candida species can be used and/or genetically modified for production of a fatty dicarboxylic acid (e.g., octanedioic acid, decanedioic acid, dodecanedioic acid, tetradecanedioic acid, hexadecanedioic acid, octadecanedioic acid, eicosanedioic acid). In some embodiments, suitable Candida species include, but are not limited to Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii and any other Candida spp. yeast described herein. Non-limiting examples of Candida spp. strains include, but are not limited to, sAA001 (ATCC20336), sAA002 (ATCC20913), sAA003 (ATCC20962), sAA496 (US2012/0077252), sAA106 (US2012/0077252), SU-2 (ura3−/ura3−), H5343 (beta oxidation blocked; U.S. Pat. No. 5,648,247) strains. Any suitable strains from Candida spp. yeast may be utilized as parental strains for genetic modification.

Yeast genera, species and strains are often so closely related in genetic content that they can be difficult to distinguish, classify and/or name. In some cases strains of C. lipolytica and Y. lipolytica can be difficult to distinguish, classify and/or name and can be, in some cases, considered the same organism. In some cases, various strains of C. tropicalis and C. viswanathii can be difficult to distinguish, classify and/or name (for example see Arie et. al., J. Gen. Appl. Microbiol., 46, 257-262 (2000). Some C. tropicalis and C. viswanathii strains obtained from ATCC as well as from other commercial or academic sources can be considered equivalent and equally suitable for the embodiments described herein. In some embodiments, some parental strains of C. tropicalis and C. viswanathii are considered to differ in name only.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable prokaryote may be selected as a host microorganism, engineered microorganism or source for a heterologous polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stbl2, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea llavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata).

Microorganisms or cells used as host organisms or source for a heterologous polynucleotide are commercially available. Microorganisms and cells described herein, and other suitable microorganisms and cells are available, for example, from Invitrogen Corporation, (Carlsbad, CA), American Type Culture Collection (Manassas, Virginia), and Agricultural Research Culture Collection (NRRL; Peoria, Illinois). Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Polymerase

A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases.

The ability to improve specificity, processivity, or other features of polymerases unnatural nucleic acids would be highly desirable in a variety of contexts where, e.g., unnatural nucleic acid incorporation is desired, including amplification, sequencing, labeling, detection, cloning, and many others. The present invention provides polymerases with modified properties for unnatural nucleic acids, methods of making such polymerases, methods of using such polymerases, and many other features that will become apparent upon a complete review of the following.

In some instances, disclosed herein includes polymerases that incorporate unnatural nucleic acids into a growing template copy, e.g., during DNA amplification. In some embodiments, polymerases can be modified such that the active site of the polymerase is modified to reduce steric entry inhibition of the unnatural nucleic acid into the active site. In some embodiments, polymerases can be modified to provide complementarity with one or more unnatural features of the unnatural nucleic acids. Such polymerases can be expressed or engineered in cells for stably incorporating a UBP into the cells. Accordingly, the invention includes compositions that include a heterologous or recombinant polymerase and methods of use thereof.

Polymerases can be modified using methods pertaining to protein engineering. For example, molecular modeling can be carried out based on crystal structures to identify the locations of the polymerases where mutations can be made to modify a target activity. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of polymerases can be used in a method or composition set forth herein including, for example, protein-based enzymes isolated from biological systems and functional variants thereof. Reference to a particular polymerase, such as those exemplified below, will be understood to include functional variants thereof unless indicated otherwise. In some embodiments, a polymerase is a wild type polymerase. In some embodiments, a polymerase is a modified, or mutant, polymerase.

Polymerases, with features for improving entry of unnatural nucleic acids into active site regions and for coordinating with unnatural nucleotides in the active site region, can also be used. In some embodiments, a modified polymerase has a modified nucleotide binding site.

In some embodiments, a modified polymerase has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the unnatural nucleic acid. In some embodiments, a modified or wild type polymerase has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified sugar. In some embodiments, a modified or wild type polymerase has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type polymerase toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type polymerase toward a nucleic acid comprising a triphosphate and/or the unnatural nucleic acid without the triphosphate. For example, a modified or wild type polymerase can have a specificity for an unnatural nucleic acid comprising a triphosphate that is at least about 10%, 20%, 30%, 40%, 50%, 60%, 70%, 80%, 90%, 95%, 97%, 98%, 99%, 99.5% 99.99% the specificity of the wild type polymerase toward the unnatural nucleic acid with a diphosphate or monophosphate, or no phosphate, or a combination thereof.

In some embodiments, a modified or wild type polymerase has a relaxed specificity for an unnatural nucleic acid. In some embodiments, a modified or wild type polymerase has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid. In some embodiments, a modified or wild type polymerase has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5% 99.99% the specificity of the wild type polymerase toward the natural nucleic acid. In some embodiments, a modified or wild type polymerase has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type polymerase toward the natural nucleic acid.

Absence of exonuclease activity can be a wild type characteristic or a characteristic imparted by a variant or engineered polymerase. For example, an exo minus Klenow fragment is a mutated version of Klenow fragment that lacks 3′ to 5′ proofreading exonuclease activity.

The method of the invention may be used to expand the substrate range of any DNA polymerase which lacks an intrinsic 3 to 5′ exonuclease proofreading activity or where a 3 to 5′ exonuclease proofreading activity has been disabled, e.g. through mutation. Examples of DNA polymerases include polA, polB (see e.g. Parrel & Loeb, Nature Struc Biol 2001) polC, polD, polY, polX and reverse transcriptases (RT) but preferably are processive, high-fidelity polymerases (PCT/GB2004/004643). In some embodiments a modified or wild type polymerase substantially lacks 3′ to 5′ proofreading exonuclease activity. In some embodiments a modified or wild type polymerase substantially lacks 3′ to 5′ proofreading exonuclease activity for an unnatural nucleic acid. In some embodiments, a modified or wild type polymerase has a 3′ to 5′ proofreading exonuclease activity. In some embodiments, a modified or wild type polymerase has a 3′ to 5′ proofreading exonuclease activity for a natural nucleic acid and substantially lacks 3′ to 5′ proofreading exonuclease activity for an unnatural nucleic acid.

In some embodiments, a modified polymerase has a 3′ to 5′ proofreading exonuclease activity that is at least about 60%, 70%, 80%, 90%, 95%, 97%, 98%, 99%, 99.5%, 99.99% the proofreading exonuclease activity of the wild type polymerase. In some embodiments, a modified polymerase has a 3′ to 5′ proofreading exonuclease activity for an unnatural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for an unnatural nucleic acid and a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to a natural nucleic acid. In some embodiments, a modified polymerase has a 3′ to 5′ proofreading exonuclease activity for a natural nucleic acid that is at least about 60%, 70%, 80%, 90%, 95%, 97%, 98%, 99%, 99.5%, 99.99% the proofreading exonuclease activity of the wild type polymerase to the natural nucleic acid.

In some embodiments, polymerases are characterized according to their rate of dissociation from nucleic acids. In some embodiments a polymerase has a relatively low dissociation rate for one or more natural and unnatural nucleic acids. In some embodiments a polymerase has a relatively high dissociation rate for one or more natural and unnatural nucleic acids. The dissociation rate is an activity of a polymerase that can be adjusted to tune reaction rates in methods set forth herein.

In some embodiments, polymerases are characterized according to their fidelity when used with a particular natural and/or unnatural nucleic acid or collections of natural and/or unnatural nucleic acid. Fidelity generally refers to the accuracy with which a polymerase incorporates correct nucleic acids into a growing nucleic acid chain when making a copy of a nucleic acid template. DNA polymerase fidelity can be measured as the ratio of correct to incorrect natural and unnatural nucleic acid incorporations when the natural and unnatural nucleic acid are present, e.g., at equal concentrations, to compete for strand synthesis at the same site in the polymerase-strand-template nucleic acid binary complex. DNA polymerase fidelity can be calculated as the ratio of (k_{cat}/Kₘ) for the natural and unnatural nucleic acid and (k_{cat}/Kₘ) for the incorrect natural and unnatural nucleic acid; where k_{cat} and Kₘ are Michaelis-Menten parameters in steady state enzyme kinetics (Fersht, A. R. (1985) Enzyme Structure and Mechanism, 2nd ed., p 350, W. H. Freeman & Co., New York., incorporated herein by reference). In some embodiments, a polymerase has a fidelity value of at least about 100, 1000, 10,000, 100,000, or 1×10⁶, with or without a proofreading activity.

In some embodiments, polymerases from native sources or variants thereof are screened using an assay that detects incorporation of an unnatural nucleic acid having a particular structure. In one example, polymerases can be screened for the ability to incorporate an unnatural nucleic acid or UBP; e.g., d5SICSTP, dNaMTP, or d5SICSTP-dNaMTP UBP. A polymerase, e.g., a heterologous polymerase, can be used that displays a modified property for the unnatural nucleic acid as compared to the wild-type polymerase. For example, the modified property can be, e.g., Kₘ, k_{cat}, Vₘₐₓ, polymerase processivity in the presence of an unnatural nucleic acid (or of a naturally occurring nucleotide), average template read-length by the polymerase in the presence of an unnatural nucleic acid, specificity of the polymerase for an unnatural nucleic acid, rate of binding of an unnatural nucleic acid, rate of product (pyrophosphate, triphosphate, etc.) release, branching rate, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the polymerase optionally has an increased rate of binding of an unnatural nucleic acid, an increased rate of product release, and/or a decreased branching rate, as compared to a wild-type polymerase.

At the same time, a polymerase can incorporate natural nucleic acids, e.g., A, C, G, and T, into a growing nucleic acid copy. For example, a polymerase optionally displays a specific activity for a natural nucleic acid that is at least about 5% as high (e.g., 5%, 10%, 25%, 50%, 75%, 100% or higher), as a corresponding wild-type polymerase and a processivity with natural nucleic acids in the presence of a template that is at least 5% as high (e.g., 5%, 10%, 25%, 50%, 75%, 100% or higher) as the wild-type polymerase in the presence of the natural nucleic acid. Optionally, the polymerase displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type polymerase.

Polymerases used herein that can have the ability to incorporate an unnatural nucleic acid of a particular structure can also be produced using a directed evolution approach. A nucleic acid synthesis assay can be used to screen for polymerase variants having specificity for any of a variety of unnatural nucleic acids. For example, polymerase variants can be screened for the ability to incorporate an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant polymerase variant. In some embodiments, such an assay is an in vivo assay, e.g., expressing a polymerase variant in a cell. Such directed evolution techniques can be used to screen variants of any suitable polymerase for activity toward any of the unnatural nucleic acids set forth herein.

Modified polymerases of the compositions described can optionally be a modified and/or recombinant (29-type DNA polymerase. Optionally, the polymerase can be a modified and/or recombinant (D29, B103, GA-1, PZA, (D15, BS32, M2Y, Nf, G1, Cp-1, PRD1, PZE, SF5, Cp-5, Cp-7, PR4, PR5, PR722, or L17 polymerase.

Nucleic acid polymerases generally useful in the invention include DNA polymerases, RNA polymerases, reverse transcriptases, and mutant or altered forms thereof. DNA polymerases and their properties are described in detail in, among other places, DNA Replication 2^{nd} edition, Kornberg and Baker, W. H. Freeman, New York, N. Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (TIi) DNA polymerase (also referred to as Vent™ DNA polymerase, Cariello et al, 1991, Polynucleotides Res, 19: 4193, New England Biolabs), 9° Nm™ DNA polymerase (New England Biolabs), Stoffel fragment, Thermo Sequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al, 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from Thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from Thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al, 1981, J Biol. Chem. 256:3112), and archaeal DP1I/DP2 DNA polymerase II (Cann et al, 1998, Proc. Natl. Acad. Sci. USA 95:14250). Both mesophilic polymerases and thermophilic polymerases are contemplated. Thermophilic DNA polymerases include, but are not limited to, ThermoSequenase®, 9° Nm™, Therminator™, Taq, Tne, Tma, Pfu, TfI, Tth, TIi, Stoffel fragment, Vent™ and Deep Vent™ DNA polymerase, KOD DNA polymerase, Tgo, JDF-3, and mutants, variants and derivatives thereof. A polymerase that is a 3′ exonuclease-deficient mutant is also contemplated. Reverse transcriptases useful in the invention include, but are not limited to, reverse transcriptases from HIV, HTLV-I, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al, CRC Crit Rev Biochem. 3:289-347(1975)). Further examples of polymerases include, but are not limited to 9° N DNA Polymerase, Taq DNA polymerase, Phusion® DNA polymerase, Pfu DNA polymerase, RB69 DNA polymerase, KOD DNA polymerase, and VentR® DNA polymerase Gardner et al. (2004) “Comparative Kinetics of Nucleotide Analog Incorporation by Vent DNA Polymerase (J. Biol. Chem., 279(12), 11834-11842; Gardner and Jack “Determinants of nucleotide sugar recognition in an archaeon DNA polymerase” Nucleic Acids Research, 27(12) 2545-2553.) Polymerases isolated from non-thermophilic organisms can be heat inactivatable. Examples are DNA polymerases from phage. It will be understood that polymerases from any of a variety of sources can be modified to increase or decrease their tolerance to high temperature conditions. In some embodiments, a polymerase can be thermophilic. In some embodiments, a thermophilic polymerase can be heat inactivatable. Thermophilic polymerases are typically useful for high temperature conditions or in thermocycling conditions such as those employed for polymerase chain reaction (PCR) techniques.

In some embodiments, the polymerase comprises Φ29, B103, GA-1, PZA, (115, BS32, M2Y, Nf, G1, Cp-1, PRD1, PZE, SF5, Cp-5, Cp-7, PR4, PR5, PR722, L17, ThermoSequenase®, 9° Nm™, Therminator™ DNA polymerase, Tne, Tma, TfI, Tth, TIi, Stoffel fragment, Vent™ and Deep Vent™ DNA polymerase, KOD DNA polymerase, Tgo, JDF-3, Pfu, Taq, T7 DNA polymerase, T7 RNA polymerase, PGB-D, UlTma DNA polymerase, E. coli DNA polymerase I, E. coli DNA polymerase III, archaeal DP1II/DP2 DNA polymerase II, 9° N DNA Polymerase, Taq DNA polymerase, Phusion® DNA polymerase, Pfu DNA polymerase, SP6 RNA polymerase, RB69 DNA polymerase, Avian Myeloblastosis Virus (AMV) reverse transcriptase, Moloney Murine Leukemia Virus (MMLV) reverse transcriptase, SuperScript® II reverse transcriptase, and SuperScript® III reverse transcriptase.

In some embodiments, the polymerase is DNA polymerase 1-Klenow fragment, Vent polymerase, Phusion® DNA polymerase, KOD DNA polymerase, Taq polymerase, T7 DNA polymerase, T7 RNA polymerase, Therminator™ DNA polymerase, POLB polymerase, SP6 RNA polymerase, E. coli DNA polymerase I, E. coli DNA polymerase III, Avian Myeloblastosis Virus (AMV) reverse transcriptase, Moloney Murine Leukemia Virus (MMLV) reverse transcriptase, SuperScript® II reverse transcriptase, or SuperScript® III reverse transcriptase.

Additionally, such polymerases can be used for DNA amplification and/or sequencing applications, including real-time applications, e.g., in the context of amplification or sequencing that include incorporation of unnatural nucleic acid residues into DNA by the polymerase. In other embodiments, the unnatural nucleic acid that is incorporated can be the same as a natural residue, e.g., where a label or other moiety of the unnatural nucleic acid is removed by action of the polymerase during incorporation, or the unnatural nucleic acid can have one or more feature that distinguishes it from a natural nucleic acid.

### Nucleotide Transporter

Nucleotide transporters (NTs) are a group of membrane transport proteins that facilitate nucleoside substrates across cell membranes and vesicles. In some embodiments, there are two types of nucleoside transporters, concentrative nucleoside transporters and equilibrative nucleoside transporters. In some instances, NTs also encompass the organic anion transporters (OAT) and the organic cation transporters (OCT). In some instances, nucleotide transporter is a nucleotide triphosphate transporter.

In some embodiments, a nucleotide triphosphate transporter (NTT) is from bacteria, plant, or algae. In some embodiments, a nucleotide triphosphate transporter is TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii).

In some embodiments, NTT is CNT1, CNT2, CNT3, ENT1, ENT2, OAT1, OAT3, or OCT1.

In some embodiments, NTT imports unnatural nucleic acids into an organism, e.g. a cell. In some embodiments, NTTs can be modified such that the nucleotide binding site of the NTT is modified to reduce steric entry inhibition of the unnatural nucleic acid into the nucleotide biding site. In some embodiments, NTTs can be modified to provide increased interaction with one or more unnatural features of the unnatural nucleic acids. Such NTTs can be expressed or engineered in cells for stably importing a UBP into the cells. Accordingly, the invention includes compositions that include a heterologous or recombinant NTT and methods of use thereof.

NTTs can be modified using methods pertaining to protein engineering. For example, molecular modeling can be carried out based on crystal structures to identify the locations of the NTTs where mutations can be made to modify a target activity or binding site. A residue identified as a target for replacement can be replaced with a residue selected using energy minimization modeling, homology modeling, and/or conservative amino acid substitutions, such as described in Bordo, et al. J Mol Biol 217: 721-729 (1991) and Hayes, et al. Proc Natl Acad Sci, USA 99: 15926-15931 (2002).

Any of a variety of NTTs can be used in a method or composition set forth herein including, for example, protein-based enzymes isolated from biological systems and functional variants thereof. Reference to a particular NTT, such as those exemplified below, will be understood to include functional variants thereof unless indicated otherwise. In some embodiments, a NTT is a wild type NTT. In some embodiments, a NTT is a modified, or mutant, NTT.

NTTs, with features for improving entry of unnatural nucleic acids into cells and for coordinating with unnatural nucleotides in the nucleotide biding region, can also be used. In some embodiments, a modified NTT has a modified nucleotide binding site. In some embodiments, a modified or wild type NTT has a relaxed specificity for an unnatural nucleic acid.

In some embodiments, a modified NTT has a specificity for an unnatural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the unnatural nucleic acid. In some embodiments, a modified or wild type NTT has a specificity for an unnatural nucleic acid comprising a modified sugar that is at least about 10%, 20%, 30%, 40%, 50%, 60%, 70%, 80%, 90%, 95%, 97%, 98%, 99%, 99.5%, 99.99% the specificity of the wild type NTT toward a natural nucleic acid and/or the unnatural nucleic acid without the modified sugar. In some embodiments, a modified or wild type NTT has a specificity for an unnatural nucleic acid comprising a modified base that is at least about 10%, 20%, 30%, 40%, 50%, 60%, 70%, 80%, 90%, 95%, 97%, 98%, 99%, 99.5%, 99.99% the specificity of the wild type NTT toward a natural nucleic acid and/or the unnatural nucleic acid without the modified base. In some embodiments, a modified or wild type polymerase has a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type NTT toward a nucleic acid comprising a triphosphate and/or the unnatural nucleic acid without the triphosphate. For example, a modified or wild type NTT can have a specificity for an unnatural nucleic acid comprising a triphosphate that is at least about 10%, 20%, 30%, 40%, 50%, 60%, 70%, 80%, 90%, 95%, 97%, 98%, 99%, 99.5%, 99.99% the specificity of the wild type NTT toward the unnatural nucleic acid with a diphosphate or monophosphate, or no phosphate, or a combination thereof.

In some embodiments, a modified or wild type NTT has a specificity for an unnatural nucleic acid and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid. In some embodiments, a modified or wild type NTT has a specificity for an unnatural nucleic acid comprising a modified sugar and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid. In some embodiments, a modified or wild type NTT has a specificity for an unnatural nucleic acid comprising a modified base and a specificity to a natural nucleic acid that is at least about 10%, 20%, 30%, 40%, 50%, 60%, 70%, 80%, 90%, 95%, 97%, 98%, 99%, 99.5%, 99.99% the specificity of the wild type NTT toward the natural nucleic acid.

NTTs can be characterized according to their rate of dissociation from nucleic acids. In some embodiments a NTT has a relatively low dissociation rate for one or more natural and unnatural nucleic acids. In some embodiments a NTT has a relatively high dissociation rate for one or more natural and unnatural nucleic acids. The dissociation rate is an activity of a NTT that can be adjusted to tune reaction rates in methods set forth herein.

NTTs from native sources or variants thereof can be screened using an assay that detects importation of an unnatural nucleic acid having a particular structure. In one example, NTTs can be screened for the ability to import an unnatural nucleic acid or UBP; e.g., d5SICSTP, dNaMTP, or d5SICSTP-dNaMTP UBP. A NTT, e.g., a heterologous NTT, can be used that displays a modified property for the unnatural nucleic acid as compared to the wild-type NTT. For example, the modified property can be, e.g., Kₘ, k_{cat}, Vₘₐₓ, NTT importation in the presence of an unnatural nucleic acid (or of a naturally occurring nucleotide), average template read-length by a cell with the NTT in the presence of an unnatural nucleic acid, specificity of the NTT for an unnatural nucleic acid, rate of binding of an unnatural nucleic acid, or rate of product release, or any combination thereof. In one embodiment, the modified property is a reduced Kₘ for an unnatural nucleic acid and/or an increased k_{cat}/Kₘ or Vₘₐₓ/Kₘ for an unnatural nucleic acid. Similarly, the NTT optionally has an increased rate of binding of an unnatural nucleic acid, an increased rate of product release, and/or an increased cell importation rate, as compared to a wild-type NTT.

At the same time, a NTT can import natural nucleic acids, e.g., A, C, G, and T, into cell. For example, a NTT optionally displays a specific importation activity for a natural nucleic acid that is at least about 5% as high (e.g., 5%, 10%, 25%, 50%, 75%, 100% or higher), as a corresponding wild-type NTT. Optionally, the NTT displays a k_{cat}/Kₘ or Vₘₐₓ/Kₘ for a naturally occurring nucleotide that is at least about 5% as high (e.g., about 5%, 10%, 25%, 50%, 75% or 100% or higher) as the wild-type NTT.

NTTs used herein that can have the ability to import an unnatural nucleic acid of a particular structure can also be produced using a directed evolution approach. A nucleic acid synthesis assay can be used to screen for NTT variants having specificity for any of a variety of unnatural nucleic acids. For example, NTT variants can be screened for the ability to import an unnatural nucleic acid or UBP; e.g., d5SICSTP, dNaMTP, or d5SICSTP-dNaMTP UBP into nucleic acids. In some embodiments, such an assay is an in vitro assay, e.g., using a recombinant NTT variant. In some embodiments, such an assay is an in vivo assay, e.g., expressing a NTT variant in a cell. Such directed evolution techniques can be used to screen variants of any suitable NTT for activity toward any of the unnatural nucleic acids set forth herein.

### Nucleic Acid Reagents & Tools

A nucleic acid reagent for use with a method, cell, or engineered microorganism described herein comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example. In some embodiments, a nucleic acid reagent or other reagent described herein is isolated or purified.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A nucleic acid or nucleic acid reagent can comprise certain elements, e.g., regulatory elements, often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

Nucleic acid reagents, e.g., expression cassettes and/or expression vectors, can include a variety of regulatory elements, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleotide triphosphate transporter nucleic acid segment. A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3″ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

As noted above, nucleic acid reagents may also comprise one or more 5′ UTR's, and one or more 3′UTR's. For example, expression vectors used in eukaryotic host cells (e.g., yeast, fungi, insect, plant, animal, human or nucleated cells) and prokaryotic host cells (e.g., virus, bacterium) can contain sequences that signal for the termination of transcription which can affect mRNA expression. These regions can be transcribed as polyadenylated segments in the untranslated portion of the mRNA encoding tissue factor protein. The 3″ untranslated regions also include transcription termination sites. In some preferred embodiments, a transcription unit comprises a polyadenylation region. One benefit of this region is that it increases the likelihood that the transcribed unit will be processed and transported like mRNA. The identification and use of polyadenylation signals in expression constructs is well established. In some preferred embodiments, homologous polyadenylation signals can be used in the transgene constructs.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR is used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

Expression of a nucleotide triphosphate transporter from an expression cassette or expression vector can be controlled by any promoter capable of expression in prokaryotic cells or eukaryotic cells. A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with a RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions.

Non-limiting examples of selective or regulatory agents that influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

Nucleic acids encoding heterologous proteins, e.g., nucleotide triphosphate transporters, can be inserted into or employed with any suitable expression system. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids).

Recombinant expression is usefully accomplished using an expression cassette that can be part of a vector, such as a plasmid. A vector can include a promoter operably linked to nucleic acid encoding a nucleotide triphosphate transporter. A vector can also include other elements required for transcription and translation as described herein. An expression cassette, expression vector, and sequences in a cassette or vector can be heterologous to the cell to which the unnatural nucleotides are contacted. For example, a nucleotide triphosphate transporter sequence can be heterologous to the cell.

A variety of prokaryotic and eukaryotic expression vectors suitable for carrying, encoding and/or expressing nucleotide triphosphate transporters can be produced. Such expression vectors include, for example, pET, pET3d, pCR2.1, pBAD, pUC, and yeast vectors. The vectors can be used, for example, in a variety of in vivo and in vitro situations. Non-limiting examples of prokaryotic promoters that can be used include SP6, T7, T5, tac, bla, trp, gal, lac, or maltose promoters. Non-limiting examples of eukaryotic promoters that can be used include constitutive promoters, e.g., viral promoters such as CMV, SV40 and RSV promoters, as well as regulatable promoters, e.g., an inducible or repressible promoter such as a tet promoter, a hsp70 promoter, and a synthetic promoter regulated by CRE. Vectors for bacterial expression include pGEX-5X-3, and for eukaryotic expression include pCIneo-CMV. Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in Verma, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

### Cloning

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) modified or wild type nucleotide triphosphate transporters and/or polymerases), by engineering a microorganism with one or more ORFs of interest, which microorganism comprises altered activities of nucleotide triphosphate transporter activity or polymerase activity.

A nucleic acid may be specifically cleaved by contacting the nucleic acid with one or more specific cleavage agents. Specific cleavage agents often will cleave specifically according to a particular nucleotide sequence at a particular site. Examples of enzyme specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, BsaI, Bsm I, BsmBI, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., Sauer, Curr. Opin. Biotech. 5:521-527 (1994)). Other examples of recombination sites include attB, attP, attL, and attR sequences, and mutants, fragments, variants and derivatives thereof, which are recognized by the recombination protein k Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. Nos. 09/517,466, and 09/732,914; U.S. Patent Publication No. US2002/0007051; and Landy, Curr. Opin. Biotech. 3:699-707 (1993)).

Examples of recombinase cloning nucleic acids are in Gateway® systems (Invitrogen, California), which include at least one recombination site for cloning desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A nucleic acid reagent sometimes contains one or more origin of replication (ORI) elements. In some embodiments, a template comprises two or more ORIs, where one functions efficiently in one organism (e.g., a bacterium) and another function efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent, e.g., an expression cassette or vector, can include nucleic acid sequence encoding a marker product. A marker product is used to determine if a gene has been delivered to the cell and once delivered is being expressed. Example marker genes include the E. coli lacZ gene which encodes β-galactosidase and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern etal., J. Molec. Appl. Genet. 1: 327 (1982)), mycophenolic acid, (Mulligan et al., Science 209: 1422 (1980)) or hygromycin, (Sugden, et al., Mol. Cell. Biol. 5: 410-413 (1985)).

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism, and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent can be of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (e.g., Biochemistry 1996 Jun. 25; 35(25):8429-38). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

In some embodiments, a kit includes a suitable packaging material to house the contents of the kit. In some cases, the packaging material is constructed by well-known methods, preferably to provide a sterile, contaminant-free environment. The packaging materials employed herein can include, for example, those customarily utilized in commercial kits sold for use with nucleic acid sequencing systems. Exemplary packaging materials include, without limitation, glass, plastic, paper, foil, and the like, capable of holding within fixed limits a component set forth herein.

The packaging material can include a label which indicates a particular use for the components. The use for the kit that is indicated by the label can be one or more of the methods set forth herein as appropriate for the particular combination of components present in the kit. For example, a label can indicate that the kit is useful for a method of synthesizing a polynucleotide or for a method of determining the sequence of a nucleic acid.

Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like.

It will be understood that not all components necessary for a particular reaction need be present in a particular kit. Rather one or more additional components can be provided from other sources. The instructions provided with a kit can identify the additional component(s) that are to be provided and where they can be obtained.

In some embodiments, a kit is provided that is useful for stably incorporating an unnatural nucleic acid into a cellular nucleic acid, e.g., using the methods provided by the present invention for preparing genetically engineered cells. In one embodiment, a kit described herein includes a genetically engineered cell and one or more unnatural nucleic acids. In another embodiment, a kit described herein includes an isolated and purified plasmid comprising a sequence selected from SEQ ID NOs: 1-4. In a further embodiment, a kit described herein includes an isolated and purified plasmid comprises a sequence of SEQ ID NO: 4, in which the W motif of SEQ ID NO:4 comprises a sequence selected from SEQ ID NOs: 5, 7, 9, 11, 13, 15, 17, 19, 21, 23, 25, or 27; and/or the Y motif of SEQ ID NO:4 comprises a sequence selected from SEQ ID NOs: 6, 8, 10, 12, 14, 16, 18, 20, 22, 24, or 26.

In additional embodiments, the kit described herein provides a cell and a nucleic acid molecule containing a heterologous gene for introduction into the cell to thereby provide a genetically engineered cell, such as expression vectors comprising the nucleic acid of any of the embodiments hereinabove described in this paragraph.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

### EXAMPLES

These examples are provided for illustrative purposes only and not to limit the scope of the claims provided herein.

### Example 1: Control of UBP Mutations in E. coli

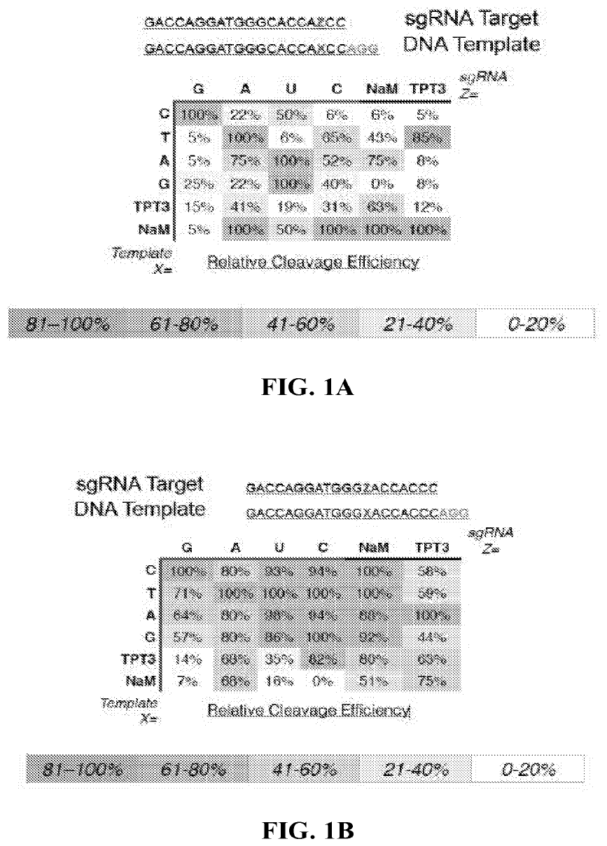

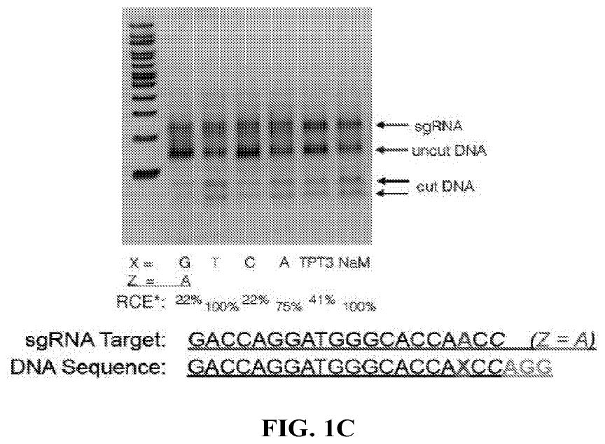

In some instances, Cas9 endonucleases are programmed by one or more single guide RNAs (sgRNAs) to create double strand breaks upstream of a protospacer adjacent motif (PAM) recognition element, which in E. coli results in rapid plasmid degradation by RecBCD and associated nucleases. Cas9/natural sgRNA complexes are less efficient at cleaving DNA sequences containing a dNaM-dTPT3 than a fully natural sequence or even a sequence containing a natural mispair, in some instances, due to the unique structure and/or lack of H-bonding potential of the unnatural nucleobases (FIGS. 1A, 1i, and 1C).

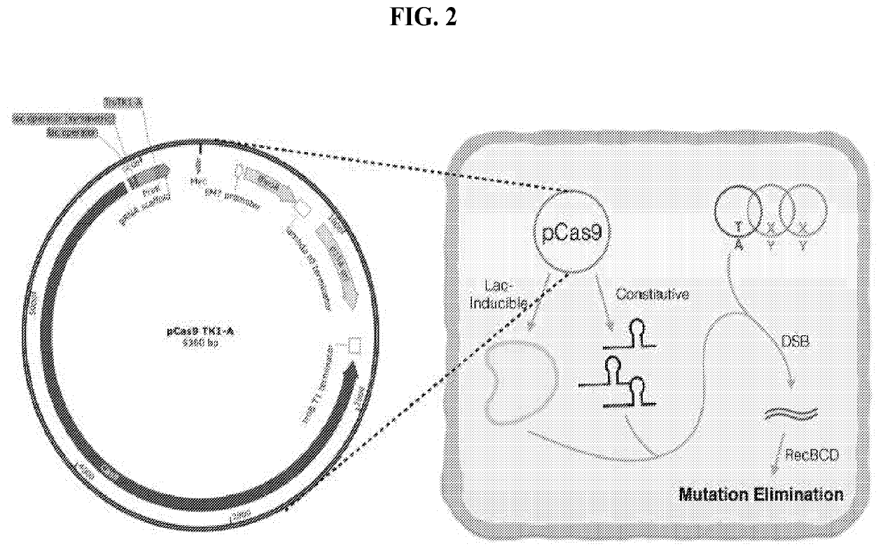

To understand whether an appropriate sgRNA used in conjunction with Cas9 degrades DNA that has lost a UBP within a cell, a plasmid containing the dNaM-dTPT3 UBP in a sequence referred to as TK-1 was constructed, as well as a plasmid pCas9/TK1-A (FIG. 2), which expresses Cas9 under an IPTG-inducible LacO promoter and an sgRNA that is fully complementary to the TK-1 sequence but contains the most common mutation, dNaM to dT, under the control of a constitutive ProK promoter. In addition, an analogous plasmid, pCas9/TruTK1-A, was constructed with a more stringent truncated TruTK1-A sgRNA which targeted the same mutation.

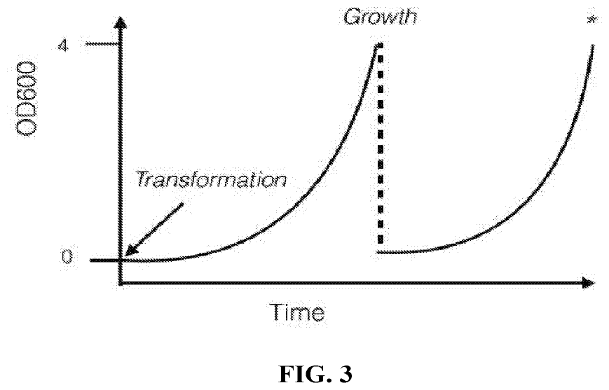

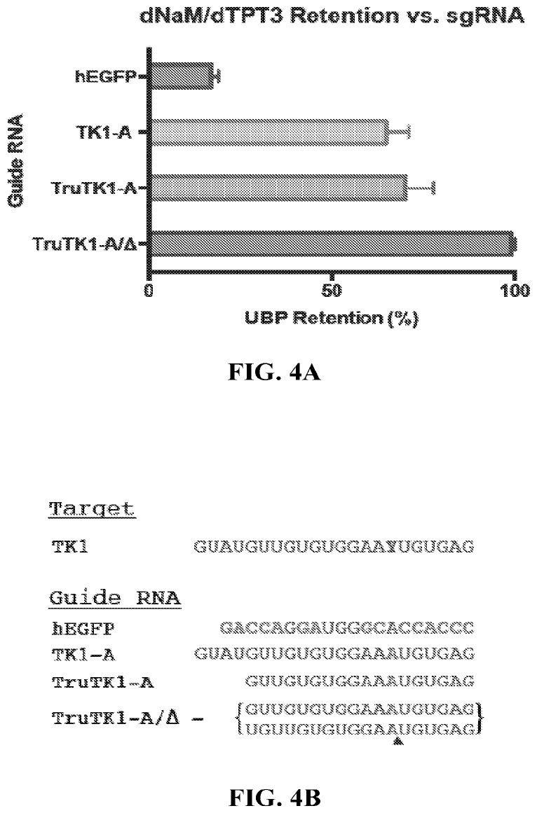

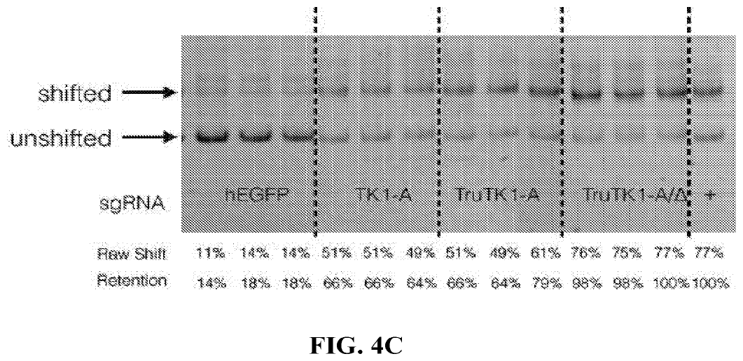

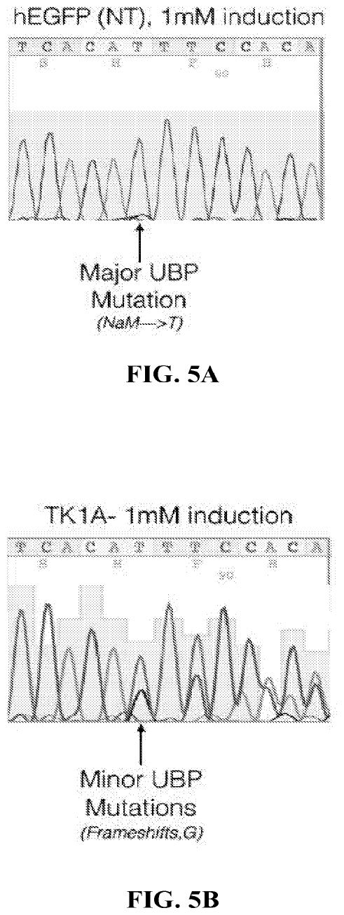

A strain of BL21(DE3) E. coli engineered to import dNaMTP and dTPT3TP via PtNTT2 was transformed with the UBP-containing plasmid and one of the pCas9 plasmids, and then grown in the presence of the unnatural triphosphates to saturation, diluted 250-fold, and grown again to saturation, all in the presence of dNaMTP and dTPT3TP supplied to the media (FIG. 3); this growth-regrowth paradigm is in some cases used for the induction of recombinant proteins. Under these conditions, dNaM-dTPT3 retention in control experiments with a scrambled sgRNA dropped to 14% after the second outgrowth (FIGS. 4A, 4B, and 4C). In contrast, in the presence of correct guide RNAs, retention was increased to 70% (TK1-A) or 77% (TruTK1-A) (FIGS. 4A, 4B, and 4C), with the remaining 30% or 23% of natural plasmids composed mainly of mutants that had lost the UBP by a single nucleotide deletion, which results in a sequence that cannot be targeted by either sgRNA. Thus, a plasmid, pCas9/TruTK1-A/A, was constructed which expresses two sgRNAs and thus targets both the major substitution (FIG. 5A) and the deletion mutation (FIG. 5B). In this case, with the same growth and regrowth assay, loss of the UBP was undetectable (FIGS. 4A, 4B, and 4C).

### Example 2: UBP Retention Enhancement is Most Effective within the Seed Region of the sgRNA

With natural DNA, Cas9/sgRNA cleavage stringency depends on the identity and distance of mismatches from the PAM recognition element. Thus, the ability of Cas9 to enforce dNaM-dTPT3 retention was assessed in either the coding or noncoding strand, at three different positions relative to the same PAM within the hGFP gene (six sequences in total; FIGS. 5A and 5B). In each case, analogous dual sgRNA cassettes were used in which the sgRNA that targets the substitution mutant varies across all four possible natural nucleotides (pCas9/hGFP-N/Δ (N=G, C, A, or U).

The same E. coli strain as in Example 1 was transformed with a UBP-containing hGFP plasmid and a pCas9/hGFP-N/Δ plasmid. UBP retention was assessed after cells reached an OD₆₀₀˜1.0. For the four cases in which the UBP was within the seed region (the region of duplex formation between the target and sgRNA, and which is the sequence most sensitive to Cas9 editing), retention was good to moderate in the absence of Cas9 induction, but increased with low levels of Cas9 expression (zero to 10 μM IPTG), regardless of the specific mutations targeted by the sgRNA. Moreover, traditional cloning via plating and inoculation obtained microgram quantities of purified plasmid with undetectable loss of the UBP. For the two cases in which the UBP was outside of the seed region, retention was poor in the absence of Cas9 induction, but increased with Cas9 expression, although this required sgRNAs targeting the major mutation and was optimal with higher levels of induction (100 μM IPTG).

### Example 3: Error-Elimination with CRISPR for Maintenance of UBPs

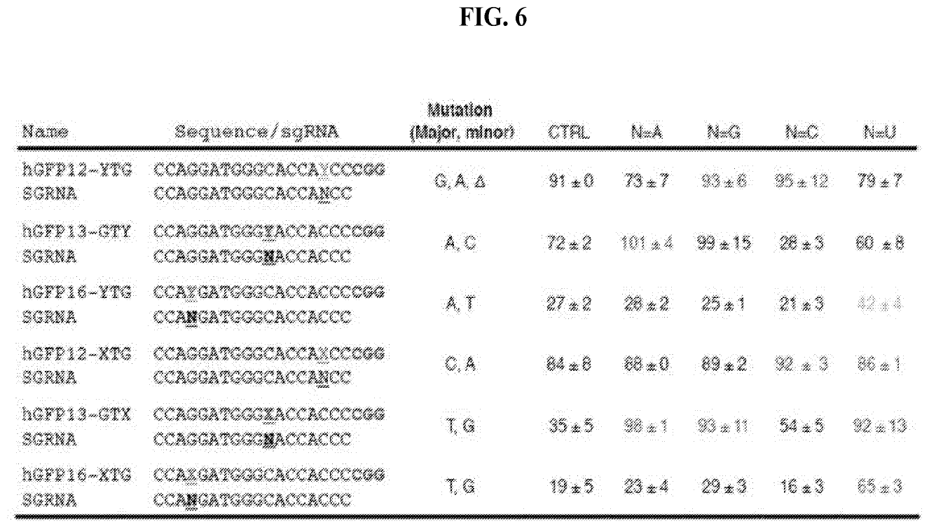

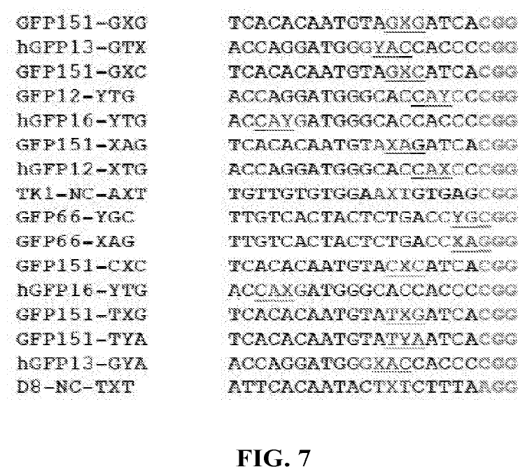

To explore the CRISPR/Cas9 editing system, in the context of its ability to enforce retention of the UBP in different sequences, a total of 16 different sequences were examined in which the dNaM of a dNaM-dTPT3 UBP was flanked by all possible nucleotides (Tables 1-3; FIG. 6). E. coli cells were transformed with a plasmid containing the UBP and a plasmid containing sgRNAs that target the major substitution mutation and the deletion mutation. A scrambled sgRNA control and low levels of Cas9 induction (10 μM TPTG) resulted in low UBP retention.

TABLE 1

| No Cas9 | | | Cas9 (+ 10 μM ITPG) | | |
|---|---|---|---|---|---|
| 3′ | % UBP | 5′ | 3′ | % UBP | 5′ |
| Nuc | Retention | Nuc | Nuc | Retention | Nuc |
| G | 36 ± 28 | G | G | 98 ± 3 | G |
| | 35 ± 5 | A | | 98 ± 1 | A |
| | 85 ± 2 | C | | 98 ± 1 | C |
| | 89 ± 3 | T | | 95 ± 12 | T |
| A | 17 ± 2 | G | A | 75 ± 3 * | G |
| | 80 | A | | 95 | A |
| | 84 ± 8 | C | | 92 ± 3 | C |
| | 90 | T | | 99 ± 5 | T |
| C | 0 | G | C | 78 ± 34 * | G |
| | 0 | A | | 78 ± 12 | A |
| | 29 ± 2 | C | | 98 ± 1 | C |
| | 27 ± 2 | T | | 60 ± 6 * | T |
| T | 0 | G | T | 47 ± 4 | G |
| | 35 ± 4 | A | | 93 ± 8 | A |
| | 72 ± 2 | C | | 101 ± 4 | C |
| | 75 | T | | 87 ± 18 | T |

* Retention with 100 μM IPTG induction of Cas9

The results demonstrated UBP was retained in the sequences tested with Cas9 and two sgRNAs. In some instances, three sequence contexts that exhibited relatively poor retention with low (10 μM IPTG) Cas9 induction (CNaMG, CNaMT, and ANaMG), were examined at higher Cas9 induction (100 μM TPTG), in which a higher UBP retention rate was observed compared to the low Cas9 induction tested above. In addition, replication (and targeting, by Cas9) of the 16 UBP-containing DNA sequences (targeting motif illustrated in Table 2) was assessed by plating onto solid media containing dNaMTP and dTPT3TP to select for single colonies, analogous to standard molecular biology practices. In some instances, selection of clonal populations purifies the UBP-containing plasmids away from those that contain errors introduced during their construction.

### Example 4: Sequences Utilized in a Method Described Herein

A plasmid described herein is illustrated by SEQ ID NO: 1. In some instances, it is referred to as pCas9-TK1-A.

SEQ ID NO: 1
ctctgcttggacggacaggatgtatgctgtggctatttaaggataactaccttgggggccattcattgattccaactccgggatctggt
cacgcagggcaaaaaagctccgttttagctcgttcctcctctggcgctccaagacgttgtgtgttcgcctcttgacattctcctcggtg
tccgagggccctgtgtgaaattgttatccgctcacaattccacacagacgtcgttgacaattaatcatcggcatagtatatcggcatag
tataatacgacaaggtgaggaactaaaccatggccaagttgaccagtgccgttccggtgctcaccgcgcgcgacgtcgccggagcggtc
gagttctggaccgaccggctcgggttctcccgggacttcgtggaggacgacttcgccggtgtggtccgggacgacgtgaccctgttcat
cagcgcggtccaggaccaggtggtgccggacaacaccctggcctgggtgtgggtgcgcggcctggacgagctgtacgccgagtggtcgg
aggtcgtgtccacgaacttccgggacgcctccgggccggccatgaccgagatcggcgagcagccgtgggggcgggagttcgccctgcgc
gacccggccggcaactgcgtgcacttcgtggccgaggagcaggactgagagctcgcttggactcctgttgatagatccagtaatgacct
cagaactccatctggatttgttcagaacgctcggttgccgccgggcgatatattggtgagaatccaagcactagtaacaacttatatcg
tatggggctgacttcaggtgctacatttgaagagataaattgcactgaaatctagtaatattttatctgattaataagatgatcttctt
gagatcgttttggtctgcgcgtaatctcttgctctgaaaacgaaaaaaccgccttgcagggcggtttttcgaaggttctctgagctacc
aactctttgaaccgaggtaactggcttggaggagcgcagtcaccaaaacttgtcctttcagtttagccttaaccggcgcatgacttcaa
gactaactcctctaaatcaattaccagtggctgctgccagtggtgcttttgcatgtctttccgggttggactcaagacgatagttaccg
gataaggcgcagcggtcggactgaacggggggttcgtgcatacagtccagcttggagcgaactgcctacccggaactgagtgtcaggcg
tggaatgagacaaacgcggccataacagcggaatgacaccggtaaaccgaaaggcaggaacaggagagcgcacgagggagccgccaggg
ggaaacgcctggtatctttatagtcctgtcgggtacgccaccactgatttgagcgtcagatttcgtgatgcttgtcaggggggcggagc
ctatggaaaaacggctttgccgcggccctctcacttccctgttaagtatcttcctggcatcttccaggaaatctccgccccgttcgtaa
gccatttccgctcgccgcagtcgaacgaccgagcgtagcgagtcagtgagcgaggaagcggaatatatcccctaggtctagggcggcgg
atttgtcctactcaggagagcgttcaccgacaaacaacagataaaacgaaaggcccagtctttcgactgagcctttcgttttatttgat
gcctctagattacaccttcctcttcttcttggggtcagccctgctgtctccaccgagctgagagaggtcgattcttgtttcatagagcc
ccgtaattgactgatgaatcagtgtggcgtccaggacctcctttgtagaggtgtaccgctttctgtctatggtggtgtcgaagtacttg
aaggctgcaggcgcgcccaagttggtcagagtaaacaagtggataatgttttctgcctgctccctgatgggcttatccctgtgcttatt
gtaagcagaaagcaccttatcgaggttagcgtcggcgaggatcactcttttggagaattcgcttatttgctcgatgatctcatcaaggt
agtgtttgtgttgttccacgaacagctgcttctgctcattatcttcgggagaccctttgagcttttcatagtggctggccagatacaag
aaattaacgtatttagagggcagtgccagctcgttacctttctgcagctcgcccgcactagcgagcattcgtaccggccgattcaagct
caaagagagagtacttgggaagcttaatgatgaggtcattagacctctttatatcctttcgcctcgagaaagtcgatggggtttttttc
gaagcttgatcgctccatgattgtgatgcccagcagttccttgacgcttagagttttttagacttccctttctccactttggccacaac
cagtacactgtaagcgactgtaggagaatcgaatccgccgtatttcttggggtcccaatcttttttgcgtgcgatcagcttgtcgctgt
tccttttcgggaggatactttccttggagaagcctccggtctgtacttcggtctattaacgatgttcacctgcggcatggacaggacct
tccggactgtcgcgaaatccctacccttgtcccacacgatttctcctgtttctccgtttgtttcgataagtggtcgcttccgaatctct
ccattggccagtgtaatctcggtcttgaaaaaattcataatattgctgtaaaagaagtacttagcggtggccttgcctatttcctgctc
agactttgcgatcattttcctaacatcgtacactttatagtctccgtaaacaaattcagattcaagcttgggatattttttgataagtg
cagtgcctaccactgcattcaggtaggcatcatgcgcatggtggtaattgttgatctctctcaccttataaaactgaaagtcctttctg
aaatctgagaccagcttagacttcagagtaataactttcacctctcgaatcagtttgtcattttcatcgtacttggtgttcatgcgtga
atcgagaatttgggccacgtgcttggtgatctggcgtgtctcaacaagctgccttttgatgaagccggctttatccaactcagacaggc
cacctcgttcagccttagtcagattatcgaacttccgttgtgtgatcagtttggcgttcagcagctgccgccaataatttttcattact
tgacaacttcttctgaggggacgttatcactcttccctctatttttatcggatcttgtcaacactttattatcaatagaatcatctttg
agaaaagactggggcacgatatgatccacgtcgtagtcggagagccgattgatgtccagttcctgatccacgtacatgtccctgccgtt
ctgcaggtagtacaggtagagcttctcattctgaagctgggtgttttcaactgggtgttccttaaggatttgggaccccagttctttta
taccctcttcaatcctcttcatcctttccctactgttcttctgtcccttctgggtagtttggttctctcgggccatctcgataacgata
ttctcgggcttatgccttcccattactttgacgagttcatccacgaccttaacggtctgcagtattccctattgatagctgggctacct
gcaagattagcgatgtgctcgtgaagactgtccccctggccagaaacttgtgctttctggatgtcctccttaaaggtgagagagtcatc
atggatcaactgcatgaagttccggttggcaaatccatcggacttaagaaaatccaggattgtctttccactctgcttgtctcggatcc
cattgatcagttttcttgacagccgcccccatcctgtatatcggcgcctcttgagctgtttcatgactttgtcgtcgaagagatgagcg
taagttttcaagcgttcttcaatcatctccctatcttcaaacaacgtaagggtgaggacaatgtcctcaagaatgtcctcgttctcctc
attgtccaggaagtccttgtctttaatgattttcaggagatcgtgatacgttcccagggatgcgttgaagcgatcctccactccgctga
tttcaacagagtcgaaacattcaatctttttgaaatagtcttctttgagctgtttcacggtaactttccggttcgtcttgaagaggagg
tccacgatagattcttctgctctccagacaggaatgctggctttctcatcccttctgtgacgtatttgaccttggtgagctcgttataa
actgtgaagtactcgtacagcagagagtgtttaggaagcaccttttcgttaggcagatttttatcaaagttagtcatcctttcgatgaa
ggactgggcagaggcccccttatccacgacttcctcgaagttccagggagtgatggtctcttctgatttgcgagtcatccacgcgaatc
tggaatttccccgggcgagggggcctacatagtagggtatccgaaatgtgaggattttctcaatcttttccctgttatctacaaaaagg
ggtagaaatcctcttgccgcctgaggatagcgtgcagttcgcccaggtgaatctggtgggggatgcttccattgtcgaaagtgcgctgt
ttgcgcaacagatcttctctgttaagctttaccagcagctcctcggtgccgtccattattccaagatgggcttaataaatttgtaaaat
tcctcctggcttgctccgccgtcaatgtatccggcgtagccatttttagactgatcgaagaaaatttccttgtacttctcaggcagttg
ctgtctgacaagggccttcagcaaagtcaagtcttggtggtgctcatcatagcgcttgatcatactagcgctcagcggagctttggtga
tctccgtgttcactcgcagaatatcactcagcagaatggcgtctgacaggttctttgccgccaaaaaaaggtctgcgtactggtcgccg
atctgggccagcagattgtcgagatcatcatcgtaggtgtctttgctcagttgaagcttggcatcttcggccaggtcgaagttagattt
aaagttgggggtcagcccgagtgacagggcgataagattaccaaacaggccgttcttcttctccccagggagctgtgcgatgaggtttt
cgagccgccgggatttggacagcctagcgctcaggattgctttggcgtcaactccggatgcgttgatcgggttctcttcgaaaagctga
ttgtaagtctgaaccagttggataaagagtttgtcgacatcgctgttgtctgggttcaggtccccctcgatgaggaagtgtccccgaaa
tttgatcatatgcgccagcgcgagatagatcaaccgcaagtcagccttatcagtactgtctacaagcttcttcctcagatgatatatgg
ttgggtacttttcatggtacgccacctcgtccacgatattgccaaagattgggtggcgctcgtgctattatcctcctccaccaaaaagg
actcctccagcctatggaagaaagagtcatccaccttagccatctcattactaaagatctcctgcaggtagcagatccgattctttctg
cgggtatatctgcgccgtgctgttcttttgagccgcgtggcttcggccgtctccccggagtcgaacaggagggcgccaatgaggttctt
ctttatgctgtggcgatcggtattgcccagaactttgaattttttgctcggcaccttgtactcgtccgtaatgacggcccagccgacgc
tgtagtgccgatatcgagcccaatggagtacttcttgtccatggtacctttctcctctttaatgaattctgtgtgaaattgttatccgc
tcacaattgaatctatcataattgtgagcgctcacaattgtaaaggttagatctaaaactagtggcagcggctaactaagcggcctgct
gactttctcgccgatcaaaaggcattttgctattaagggattgacgagggcgtatctgcgcagtaagatgcgccccgcattGTATGTTG
TGTGGAAATGTGAGgttttagagctagaaatagcaagttaaaataaggctagtccgttatcaacttgaaaaagtggcaccgagtcggtg
ctttttttaattcgaaaagcctgctcaacgagcaggcattaggtcgacagttcataggtgattgctcaggacatttctgttagaaggaa
tcgttaccttacttaccttacgcacaagagttccgtagctgttcaagtttgtgtttcaactgttctcgtcgtttccgcaacaagtcctc
ttcagaaatgagcttttgctc
A plasmid described herein is illustrated by SEQ ID NO: 2. In some instances, it is
referred to as pCas9-TruTK1-A.

SEQ ID NO: 2
ctctgcttggacggacaggatgtatgctgtggctatttaaggataactaccttgggggccattcattgattccaactccgggatctggt
cacgcagggcaaaaaagctccgttttagctcgttcctcctctggcgctccaagacgttgtgtgttcgcctcttgacattctcctcggtg
tccgagggccctgtgtgaaattgttatccgctcacaattccacacagacgtcgttgacaattaatcatcggcatagtatatcggcatag
tataatacgacaaggtgaggaactaaaccatggccaagttgaccagtgccgttccggtgctcaccgcgcgcgacgtcgccggagcggtc
gagttctggaccgaccggctcgggttctcccgggacttcgtggaggacgacttcgccggtgtggtccgggacgacgtgaccctgttcat
cagcgcggtccaggaccaggtggtgccggacaacaccctggcctgggtgtgggtgcgcggcctggacgagctgtacgccgagtggtcgg
aggtcgtgtccacgaacttccgggacgcctccgggccggccatgaccgagatcggcgagcagccgtgggggcgggagttcgccctgcgc
gacccggccggcaactgcgtgcacttcgtggccgaggagcaggactgagagctcgcttggactcctgttgatagatccagtaatgacct
cagaactccatctggatttgttcagaacgctcggttgccgccgggcgatatattggtgagaatccaagcactagtaacaacttatatcg
tatggggctgacttcaggtgctacatttgaagagataaattgcactgaaatctagtaatattttatctgattaataagatgatcttctt
gagatcgttttggtctgcgcgtaatctcttgctctgaaaacgaaaaaaccgccttgcagggcggtttttcgaaggttctctgagctacc
aactctttgaaccgaggtaactggcttggaggagcgcagtcaccaaaacttgtcctttcagtttagccttaaccggcgcatgacttcaa
gactaactcctctaaatcaattaccagtggctgctgccagtggtgcttttgcatgtctttccgggttggactcaagacgatagttaccg
gataaggcgcagcggtcggactgaacggggggttcgtgcatacagtccagcttggagcgaactgcctacccggaactgagtgtcaggcg
tggaatgagacaaacgcggccataacagcggaatgacaccggtaaaccgaaaggcaggaacaggagagcgcacgagggagccgccaggg
ggaaacgcctggtatctttatagtcctgtcgggtacgccaccactgatttgagcgtcagatttcgtgatgcttgtcaggggggcggagc
ctatggaaaaacggctttgccgcggccctctcacttccctgttaagtatcttcctggcatcttccaggaaatctccgccccgttcgtaa
gccatttccgctcgccgcagtcgaacgaccgagcgtagcgagtcagtgagcgaggaagcggaatatatcccctaggtctagggcggcgg
atttgtcctactcaggagagcgttcaccgacaaacaacagataaaacgaaaggcccagtctttcgactgagcctttcgttttatttgat
gcctctagattacaccttcctcttcttcttggggtcagccctgctgtctccaccgagctgagagaggtcgattcttgtttcatagagcc
ccgtaattgactgatgaatcagtgtggcgtccaggacctcctttgtagaggtgtaccgctttctgtctatggtggtgtcgaagtacttg
aaggctgcaggcgcgcccaagttggtcagagtaaacaagtggataatgttttctgcctgctccctgatgggcttatccctgtgcttatt
gtaagcagaaagcaccttatcgaggttagcgtcggcgaggatcactcttttggagaattcgcttatttgctcgatgatctcatcaaggt
agtgtttgtgttgttccacgaacagctgcttctgctcattatcttcgggagaccctttgagcttttcatagtggctggccagatacaag
aaattaacgtatttagagggcagtgccagctcgttacctttctgcagctcgcccgcactagcgagcattcgtttccggccgattcaagc
tcaaagagagagtacttgggaagcttaatgatgaggtcattagacctctttatatcctttcgcctcgagaaagtcgatggggttttttt
cgaagcttgatcgctccatgattgtgatgcccagcagttccttgacgcttagagttttttagacttccctttctccactttggccacaa
ccagtacactgtaagcgactgtaggagaatcgaatccgccgtatttcttggggtcccaatcttttttgcgtgcgatcagcttgtcgctg
ttccttttcgggaggatactttccttggagaagcctccggtctgtacttcggtctattaacgatgttcacctgcggcatggacaggacc
ttccggactgtcgcgaaatccctacccttgtcccacacgatttctcctgtttctccgtttgtttcgataagtggtcgcttccgaatctc
tccattggccagtgtaatctcggtcttgaaaaaattcataatattgctgtaaaagaagtacttagcggtggccttgcctatttcctgct
cagactttgcgatcattttcctaacatcgtacactttatagtctccgtaaacaaattcagattcaagcttgggatattttttgataagt
gcagtgcctaccactgcattcaggtaggcatcatgcgcatggtggtaattgttgatctctctcaccttataaaactgaaagtcctttct
gaaatctgagaccagcttagacttcagagtaataactttcacctctcgaatcagtttgtcattttcatcgtacttggtgttcatgcgtg
aatcgagaatttgggccacgtgcttggtgatctggcgtgtctcaacaagctgccttttgatgaagccggctttatccaactcagacagg
ccacctcgttcagccttagtcagattatcgaacttccgttgtgtgatcagtttggcgttcagcagctgccgccaataatttttcattac
ttgacaacttcttctgaggggacgttatcactcttccctctatttttatcggatcttgtcaacactttattatcaatagaatcatcttt
gagaaaagactggggcacgatatgatccacgtcgtagtcggagagccgattgatgtccagttcctgatccacgtacatgtccctgccgt
tctgcaggtagtacaggtagagcttctcattctgaagctgggtgttttcaactgggtgttccttaaggatttgggaccccagttctttt
ataccctcttcaatcctcttcatcctttccctactgttcttctgtcccttctgggtagtttggttctctcgggccatctcgataacgat
attctcgggcttatgccttcccattactttgacgagttcatccacgaccttaacggtctgcagtattccctattgatagctgggctacc
tgcaagattagcgatgtgctcgtgaagactgtccccctggccagaaacttgtgctttctggatgtcctccttaaaggtgagagagtcat
catggatcaactgcatgaagttccggttggcaaatccatcggacttaagaaaatccaggattgtctttccactctgcttgtctcggatc
ccattgatcagttttcttgacagccgcccccatcctgtatatcggcgcctcttgagctgtttcatgactttgtcgtcgaagagatgagc
gtaagttttcaagcgttcttcaatcatctccctatcttcaaacaacgtaagggtgaggacaatgtcctcaagaatgtcctcgttctcct
cattgtccaggaagtccttgtctttaatgattttcaggagatcgtgatacgttcccagggatgcgttgaagcgatcctccactccgctg
atttcaacagagtcgaaacattcaatctttttgaaatagtcttctttgagctgtttcacggtaactttccggttcgtcttgaagaggag
gtccacgatagattcttctgctctccagacaggaatgctggctttctcatcccttctgtgacgtatttgaccttggtgagctcgttata
aactgtgaagtactcgtacagcagagagtgtttaggaagcaccttttcgttaggcagatttttatcaaagttagtcatcctttcgatga
aggactgggcagaggcccccttatccacgacttcctcgaagttccagggagtgatggtctcttctgatttgcgagtcatccacgcgaat
ctggaatttccccgggcgagggggcctacatagtagggtatccgaaatgtgaggattttctcaatcttttccctgttatctacaaaaag
gggtagaaatcctcttgccgcctgaggatagcgtgcagttcgcccaggtgaatctggtgggggatgcttccattgtcgaaagtgcgctg
tttgcgcaacagatcttctctgttaagctttaccagcagctcctcggtgccgtccattattccaagatgggcttaataaatttgtaaaa
ttcctcctggcttgctccgccgtcaatgtatccggcgtagccatttttagactgatcgaagaaaatttccttgtacttctcaggcagtt
gctgtctgacaagggccttcagcaaagtcaagtcttggtggtgctcatcatagcgcttgatcatactagcgctcagcggagctttggtg
atctccgtgttcactcgcagaatatcactcagcagaatggcgtctgacaggttctttgccgccaaaaaaaggtctgcgtactggtcgcc
gatctgggccagcagattgtcgagatcatcatcgtaggtgtctttgctcagttgaagcttggcatcttcggccaggtcgaagttagatt
taaagttgggggtcagcccgagtgacagggcgataagattaccaaacaggccgttcttcttctccccagggagctgtgcgatgaggttt
tcgagccgccgggatttggacagcctagcgctcaggattgctttggcgtcaactccggatgcgttgatcgggttctcttcgaaaagctg
attgtaagtctgaaccagttggataaagagtttgtcgacatcgctgttgtctgggttcaggtccccctcgatgaggaagtgtccccgaa
atttgatcatatgcgccagcgcgagatagatcaaccgcaagtcagccttatcagtactgtctacaagcttcttcctcagatgatatatg
gttgggtacttttcatggtacgccacctcgtccacgatattgccaaagattgggtggcgctcgtgctattatcctcctccaccaaaaag
gactcctccagcctatggaagaaagagtcatccaccttagccatctcattactaaagatctcctgcaggtagcagatccgattctttct
gcgggtatatctgcgccgtgctgttcttttgagccgcgtggcttcggccgtctccccggagtcgaacaggagggcgccaatgaggttct
tctttatgctgtggcgatcggtattgcccagaactttgaattttttgctcggcaccttgtactcgtccgtaatgacggcccagccgacg
ctgtagtgccgatatcgagcccaatggagtacttcttgtccatggtacctttctcctctttaatgaattctgtgtgaaattgttatccg
ctcacaattgaatctatcataattgtgagcgctcacaattgtaaaggttagatctaaaactagtggcagcggctaactaagcggcctgc
tgactttctcgccgatcaaaaggcattttgctattaagggattgacgagggcgtatctgcgcagtaagatgcgccccgcattGTTGTGT
GGAAATGTGAGgttttagagctagaaatagcaagttaaaataaggctagtccgttatcaacttgaaaaagtggcaccgagtcggtgctt
tttttaattcgaaaagcctgctcaacgagcaggcttttttggtcgacagttcataggtgattgctcaggacatttctgttagaaggaat
cgttttccttacttttccttacgcacaagagttccgtagctgttcaagtttgtgtttcaactgttctcgtcgtttccgcaacaagtcct
cttcagaaatgagcttttgctc

A plasmid described herein is illustrated by SEQ ID NO: 3. In some instances, it is referred to as pCas9-TruTK1-A/Δ.

SEQ ID NO: 3
ctctgcttggacggacaggatgtatgctgtggctatttaaggataactaccttgggggccattcattgattccaactccgggatctggt
cacgcagggcaaaaaagctccgttttagctcgttcctcctctggcgctccaagacgttgtgtgttcgcctcttgacattctcctcggtg
tccgagggccctgtgtgaatttgttatccgctcacaattccacacagacgtcgttgacaattaatcatcggcatagtatatcggcatag
tataatacgacaaggtgaggaactaaaccatggccaagttgaccagtgccgttccggtgctcaccgcgcgcgacgtcgccggagcggtc
gagttctggaccgaccggctcgggttctcccgggacttcgtggaggacgacttcgccggtgtggtccgggacgacgtgaccctgttcat
cagcgcggtccaggaccaggtggtgccggacaacaccctggcctgggtgtgggtgcgcggcctggacgagctgtacgccgagtggtcgg
aggtcgtgtccacgaacttccgggacgcctccgggccggccatgaccgagatcggcgagcagccgtgggggcgggagttcgccctgcgc
gacccggccggcaactgcgtgcacttcgtggccgaggagcaggactgagagctcgcttggactcctgttgatagatccagtaatgacct
cagaactccatctggatttgttcagaacgctcggttgccgccgggcgttttttattggtgagaatccaagcactagtaacaacttatat
cgtatggggctgacttcaggtgctacatttgaagagataaattgcactgaaatctagtaatattttatctgattaataagatgatcttc
ttgagatcgttttggtctgcgcgtaatctcttgctctgaaaacgaaaaaaccgccttgcagggcggtattcgaaggttctctgagctac
caactctagaaccgaggtaactggcttggaggagcgcagtcaccaaaacttgtcctttcagtttagccttaaccggcgcatgacttcaa
gactaactcctctaaatcaattaccagtggctgctgccagtggtgcttttgcatgtctttccgggttggactcaagacgatagttaccg
gataaggcgcagcggtcggactgaacggggggttcgtgcatacagtccagcttggagcgaactgcctacccggaactgagtgtcaggcg
tggaatgagacaaacgcggccataacagcggaatgacaccggtaaaccgaaaggcaggaacaggagagcgcacgagggagccgccaggg
ggaaacgcctggtatctttatagtcctgtcgggtacgccaccactgatttgagcgtcagatttcgtgatgcttgtcaggggggcggagc
ctatggaaaaacggctttgccgcggccctctcacttccctgttaagtatcttcctggcatcttccaggaaatctccgccccgttcgtaa
gccatttccgctcgccgcagtcgaacgaccgagcgtagcgagtcagtgagcgaggaagcggaatatatcccctaggtctagggcggcgg
atttgtcctactcaggagagcgttcaccgacaaacaacagataaaacgaaaggcccagtctttcgactgagcctttcgttttatttgat
gcctctagattacaccttcctcttcttcttggggtcagccctgctgtctccaccgagctgagagaggtcgattcttgtttcatagagcc
ccgtaattgactgatgaatcagtgtggcgtccaggacctcctttgtagaggtgtaccgctttctgtctatggtggtgtcgaagtacttg
aaggctgcaggcgcgcccaagttggtcagagtaaacaagtggataatgttttctgcctgctccctgatgggcttatccctgtgcttatt
gtaagcagaaagcaccttatcgaggttagcgtcggcgaggatcactcttttggagaattcgcttatttgctcgatgatctcatcaaggt
agtgtttgtgttgttccacgaacagctgcttctgctcattatcttcgggagaccctttgagcttttcatagtggctggccagatacaag
aaattaacgtatttagagggcagtgccagctcgttacctttctgcagctcgcccgcactagcgagcattcgtttccggccgattcaagc
tcaaagagagagtacttgggaagcttaatgatgaggtcattagacctctttatatcctttcgcctcgagaaagtcgatggggttttttt
cgaagcttgatcgctccatgattgtgatgcccagcagttccttgacgcttagagttttttagacttccctttctccactttggccacaa
ccagtacactgtaagcgactgtaggagaatcgaatccgccgtatttcttggggtcccaatcttttttgcgtgcgatcagcttgtcgctg
ttccttttcgggaggatactttccttggagaagcctccggtctgtacttcggtctattaacgatgttcacctgcggcatggacaggacc
ttccggactgtcgcgaaatccctacccttgtcccacacgatttctcctgtttctccgtttgtttcgataagtggtcgcttccgaatctc
tccattggccagtgtaatctcggtcttgaaaaaattcataatattgctgtaaaagaagtacttagcggtggccttgcctatttcctgct
cagactttgcgatcattttcctaacatcgtacactttatagtctccgtaaacaaattcagattcaagcttgggatattttttgataagt
gcagtgcctaccactgcattcaggtaggcatcatgcgcatggtggtaattgttgatctctctcaccttataaaactgaaagtcctttct
gaaatctgagaccagcttagacttcagagtaataactttcacctctcgaatcagtttgtcattttcatcgtacttggtgttcatgcgtg
aatcgagaatttgggccacgtgcttggtgatctggcgtgtctcaacaagctgccttttgatgaagccggctttatccaactcagacagg
ccacctcgttcagccttagtcagattatcgaacttccgttgtgtgatcagtttggcgttcagcagctgccgccaataatttttcattac
ttgacaacttcttctgaggggacgttatcactcttccctctatttttatcggatcttgtcaacactttattatcaatagaatcatcttt
gagaaaagactggggcacgatatgatccacgtcgtagtcggagagccgattgatgtccagttcctgatccacgtacatgtccctgccgt
tctgcaggtagtacaggtagagcttctcattctgaagctgggtgttttcaactgggtgttccttaaggatttgggaccccagttctttt
ataccctcttcaatcctcttcatcctttccctactgttcttctgtcccttctgggtagtttggttctctcgggccatctcgataacgat
attctcgggcttatgccttcccattactttgacgagttcatccacgaccttaacggtctgcagtattccctattgatagctgggctacc
tgcaagattagcgatgtgctcgtgaagactgtccccctggccagaaacttgtgctttctggatgtcctccttaaaggtgagagagtcat
catggatcaactgcatgaagttccggttggcaaatccatcggacttaagaaaatccaggattgtctttccactctgcttgtctcggatc
ccattgatcagttttcttgacagccgcccccatcctgtatatcggcgcctcttgagctgtttcatgactttgtcgtcgaagagatgagc
gtaagttttcaagcgttcttcaatcatctccctatcttcaaacaacgtaagggtgaggacaatgtcctcaagaatgtcctcgttctcct
cattgtccaggaagtccttgtctttaatgattttcaggagatcgtgatacgttcccagggatgcgttgaagcgatcctccactccgctg
atttcaacagagtcgaaacattcaatctttttgaaatagtcttctttgagctgttttcacggtaactttccggttcgtcttgaagagga
ggtccacgatagctttcttctgctctccagacaggaatgctggctttctcatcccttctgtgacgtatttgaccttggtgagctcgtta
taaactgtgaagtactcgtacagcagagagtgttttaggaagcaccttttcgttaggcagatttttatcaaagttagtcatccttttcg
atgaaggactgggcagaggcccccttatccacgacttcctcgaagttccagggagtgatggtctcttctgatttgcgagtcatccacgc
gaatctggaatttccccgggcgagggggcctacatagtagggtatccgaaatgtgaggattttctcaatcttttccctgttatctacaa
aaaggggtagaaatcctcttgccgcctgaggatagcgtgcagttcgcccaggtgaatctggtgggggatgcttccattgtcgaaagtgc
gctgtttgcgcaacagatcttctctgttaagctttaccagcagctcctcggtgccgtccattattccaagatgggcttaataaatttgt
aaaattcctcctggcttgctccgccgtcaatgtatccggcgtagccatttttagactgatcgaagaaaatttccttgtacttctcaggc
agttgctgtctgacaagggccttcagcaaagtcaagtcttggtggtgctcatcatagcgcttgatcatactagcgctcagcggagcttt
ggtgatctccgtgttcactcgcagaatatcactcagcagaatggcgtctgacaggttcttttgccgccaaaaaaaggtctgcgtactgg
tcgccgatctgggccagcagattgtcgagatcatcatcgtaggtgtctttgctcagttgaagcttggcatcttcggccaggtcgaagtt
agatttaaagttgggggtcagcccgagtgacagggcgataagattaccaaacaggccgttcttcttctccccagggagctgtgcgatga
ggtttttcgagccgccgggatttggacagcctagcgctcaggattgattggcgtcaactccggatgcgttgatcgggttctcttcgaaa
agctgattgtaagtctgaaccagttggataaagagtttgtcgacatcgctgttgtctgggttcaggtccccctcgatgaggaagtgtcc
ccgaaatttgatcatatgcgccagcgcgagatagatcaaccgcaagtcagccttatcagtactgtctacaagcttcttcctcagatgat
atatggttgggtacttttcatggtacgccacctcgtccacgatattgccaaagattgggtggcgctcgtgctattatcctcctccacca
aaaaggactcctccagcctatggaagaaagagtcatccaccttagccatctcattactaaagatctcctgcaggtagcagatccgattc
ttttctgcgggtatatctgcgccgtgctgttcttttgagccgcgtggcttcggcGgtTtccccggagtcgaacaggagggcgccaatga
ggttcttctttatgctgtggcgatcggtattgcccagaactttgaattattgctcggcaccttgtactcgtccgtaatgacggcccagc
cgacgctgtttgtgccgatatcgagcccaatggagtacttcttgtccatgggtaccttttctcctctttaatgaattctgtgtgaaatt
gttatccgctcacaattgaatctatcataattgtgagcgctcacaattgtaaaggtttagatctaaaactagtggcagcggctaactaa
gcggcctgctgactactcgccgatcaaaaggcattagctattaagggattgacgagggcgtatctgcgcagtaagaTGCGgcattGTTG
TGTGGAAATGTGAGgttttagagctagaaatagcaagttaaaataaggctagtccgttatcaacttgaaaaagtggcaccgagtcggtg
ctttttttaattcgaaaagcgctcaacgagcaggcttttttggtcgacagACAGtagtggcagcggctaactaagcggcctgctgacta
ctcgccgatcaaaaggcattagctattaagggattgacgagggcgtatctgcgcagtaagatgcgccccgcattTGTTGTGTGGAATGT
GAGgttttagagctagaaatagcaagttaaaataaggctagtccgttatcaacttgaaaaagtggcaccgagtcggtgctttttttaat
tcgaaaagcctgctcaacgagcaggcttttttggtcgacagttcataggtgattgctcaggacatttctgttagaaggaatcgttttcc
ttacttttccttacgcacaagagttccgtagctgttcaagtttgtgtttcaactgttctcgtcgtttccgcaacaagtcctcttcagaa
atgagcttttgctc

A plasmid described herein is illustrated by SEQ ID NO: 4. In some instances, it is referred to as pCas9-hGFP-N/Δ master sequence.

SEQ ID NO: 4
ctctgcttggacggacaggatgtatgctgtggctatttaaggataactaccttgggggccattcattgattccaactccgggatctggt
cacgcagggcaaaaaagctccgttttagctcgttcctcctctggcgctccaagacgttgtgtgttcgcctcttgacattctcctcggtg
tccgagggccctgtgtgaatttgttatccgctcacaattccacacagacgtcgttgacaattaatcatcggcatagtatatcggcatag
tataatacgacaaggtgaggaactaaaccatggccaagttgaccagtgccgttccggtgctcaccgcgcgcgacgtcgccggagcggtc
gagttctggaccgaccggctcgggttctcccgggacttcgtggaggacgacttcgccggtgtggtccgggacgacgtgaccctgttcat
cagcgcggtccaggaccaggtggtgccggacaacaccctggcctgggtgtgggtgcgcggcctggacgagctgtacgccgagtggtcgg
aggtcgtgtccacgaacttccgggacgcctccgggccggccatgaccgagatcggcgagcagccgtgggggcgggagttcgccctgcgc
gacccggccggcaactgcgtgcacttcgtggccgaggagcaggactgagagctcgcttggactcctgttgatagatccagtaatgacct
cagaactccatctggatttgttcagaacgctcggttgccgccgggcgttttttattggtgagaatccaagcactagtaacaacttatat
cgtatggggctgacttcaggtgctacatttgaagagataaattgcactgaaatctagtaatattttatctgattaataagatgatcttc
ttgagatcgttttggtctgcgcgtaatctcttgctctgaaaacgaaaaaaccgccttgcagggcggtattcgaaggttctctgagctac
caactctagaaccgaggtaactggcttggaggagcgcagtcaccaaaacttgtcctttcagtttagccttaaccggcgcatgacttcaa
gactaactcctctaaatcaattaccagtggctgctgccagtggtgcttttgcatgtctttccgggttggactcaagacgatagttaccg
gataaggcgcagcggtcggactgaacggggggttcgtgcatacagtccagcttggagcgaactgcctacccggaactgagtgtcaggcg
tggaatgagacaaacgcggccataacagcggaatgacaccggtaaaccgaaaggcaggaacaggagagcgcacgagggagccgccaggg
ggaaacgcctggtatctttatagtcctgtcgggtacgccaccactgatttgagcgtcagatttcgtgatgcttgtcaggggggcggagc
ctatggaaaaacggctttgccgcggccctctcacttccctgttaagtatcttcctggcatcttccaggaaatctccgccccgttcgtaa
gccatttccgctcgccgcagtcgaacgaccgagcgtagcgagtcagtgagcgaggaagcggaatatatcccctaggtctagggcggcgg
atttgtcctactcaggagagcgttcaccgacaaacaacagataaaacgaaaggcccagtctttcgactgagcctttcgttttatttgat
gcctctagattacaccttcctcttcttcttggggtcagccctgctgtctccaccgagctgagagaggtcgattcttgtttcatagagcc
ccgtaattgactgatgaatcagtgtggcgtccaggacctcctttgtagaggtgtaccgctttctgtctatggtggtgtcgaagtacttg
aaggctgcaggcgcgcccaagttggtcagagtaaacaagtggataatgttttctgcctgctccctgatgggcttatccctgtgcttatt
gtaagcagaaagcaccttatcgaggttagcgtcggcgaggatcactcttttggagaattcgcttatttgctcgatgatctcatcaaggt
agtgtttgtgttgttccacgaacagctgcttctgctcattatcttcgggagaccctttgagcttttcatagtggctggccagatacaag
aaattaacgtatttagagggcagtgccagctcgttacctttctgcagctcgcccgcactagcgagcattcgtttccggccgattcaagc
tcaaagagagagtacttgggaagcttaatgatgaggtcattagacctctttatatcctttcgcctcgagaaagtcgatggggttttttt
cgaagcttgatcgctccatgattgtgatgcccagcagttccttgacgcttttgagttttttagacttccctttctccactttggccaca
accagtacactgtaagcgactgtaggagaatcgaatccgccgtatttcttggggtcccaatcttttttgcgtgcgatcagcttgtcgct
gttccttttcgggaggatactttccttggagaagcctccggtctgtacttcggtctattaacgatgttcacctgcggcatggacaggac
cttccggactgtcgcgaaatccctacccttgtcccacacgatttctcctgtttctccgtttgtttcgataagtggtcgcttccgaatct
ctccattggccagtgtaatctcggtcttgaaaaaattcataatattgctgtaaaagaagtacttagcggtggccttgcctatttcctgc
tcagactttgcgatcattttcctaacatcgtacactttatagtctccgtaaacaaattcagattcaagcttgggatattttttgataag
tgcagtgcctaccactgcattcaggtaggcatcatgcgcatggtggtaattgttgatctctctcaccttataaaactgaaagtcctttc
tgaaatctgagaccagcttagacttcagagtaataactttcacctctcgaatcagtttgtcattttcatcgtacttggtgttcatgcgt
gaatcgagaatttgggccacgtgcttggtgatctggcgtgtctcaacaagctgccttttgatgaagccggctttatccaactcagacag
gccacctcgttcagccttagtcagattatcgaacttccgttgtgtgatcagtttggcgttcagcagctgccgccaataatttttcatta
cttgacaacttcttctgaggggacgttatcactcttccctctatttttatcggatcttgtcaacactttattatcaatagaatcatctt
tgagaaaagactggggcacgatatgatccacgtcgtagtcggagagccgattgatgtccagttcctgatccacgtacatgtccctgccg
ttctgcaggtagtacaggtagagcttctcattctgaagctgggtgttttcaactgggtgttccttaaggatttgggaccccagttcttt
tataccctcttcaatcctcttcatcctttccctactgttcttctgtcccttctgggtagtttggttctctcgggccatctcgataacga
tattctcgggcttatgccttcccattactttgacgagttcatccacgaccttaacggtctgcagtattccctttttgatagctgggcta
cctgcaagattagcgatgtgctcgtgaagactgtccccctggccagaaacttgtgctttctggatgtcctccttaaaggtgagagagtc
atcatggatcaactgcatgaagttccggttggcaaatccatcggacttaagaaaatccaggattgtctttccactctgcttgtctcgga
tcccattgatcagttttcttgacagccgcccccatcctgtatatcggcgcctcttgagctgtttcatgactttgtcgtcgaagagatga
gcgtaagttttcaagcgttcttcaatcatctccctatcttcaaacaacgtaagggtgaggacaatgtcctcaagaatgtcctcgttctc
ctcattgtccaggaagtccttgtctttaatgattttcaggagatcgtgatacgttcccagggatgcgttgaagcgatcctccactccgc
tgatttcaacagagtcgaaacattcaatctttttgaaatagtcttctttgagctgtttcacggtaactttccggttcgtcttgaagagg
aggtccacgatagattcttctgctctccagacaggaatgctggctttctcatcccttctgtgacgtatttgaccttggtgagctcgtta
taaactgtgaagtactcgtacagcagagagtgtttaggaagcaccttttcgttaggcagatttttatcaaagttagtcatcctttcgat
gaaggactgggcagaggcccccttatccacgacttcctcgaagttccagggagtgatggtctcttctgatttgcgagtcatccacgcga
atctggaatttccccgggcgagggggcctacatagtagggtatccgaaatgtgaggattttctcaatcttttccctgttatctacaaaa
aggggtagaaatcctcttgccgcctgaggatagcgtgcagttcgcccaggtgaatctggtgggggatgcttccattgtcgaaagtgcgc
tgtttgcgcaacagatcttctctgttaagctttaccagcagctcctcggtgccgtccattattccaagatgggcttaataaatttgtaa
aattcctcctggcttgctccgccgtcaatgtatccggcgtagccatttttagactgatcgaagaaaatttccttgtacttctcaggcag
ttgctgtctgacaagggccttcagcaaagtcaagtcttggtggtgctcatcatagcgcttgatcatactagcgctcagcggagctttgg
tgatctccgtgttcactcgcagaatatcactcagcagaatggcgtctgacaggttctttgccgccaaaaaaaggtctgcgtactggtcg
ccgatctgggccagcagattgtcgagatcatcatcgtaggtgtctttgctcagttgaagcttggcatcttcggccaggtcgaagttaga
tttaaagttgggggtcagcccgagtgacagggcgataagattaccaaacaggccgttcttcttctccccagggagctgtgcgatgaggt
tttcgagccgccgggatttggacagcctagcgctcaggattgctttggcgtcaactccggatgcgttgatcgggttctcttcgaaaagc
tgattgtaagtctgaaccagttggataaagagtttgtcgacatcgctgttgtctgggttcaggtccccctcgatgaggaagtgtccccg
aaatttgatcatatgcgccagcgcgagatagatcaaccgcaagtcagccttatcagtactgtctacaagcttcttcctcagatgatata
tggttgggtacttttcatggtacgccacctcgtccacgatattgccaaagattgggtggcgctcgtgctattatcctcctccaccaaaa
aggactcctccagcctatggaagaaagagtcatccaccttagccatctcattactaaagatctcctgcaggtagcagatccgattcttt
ctgcgggtatatctgcgccgtgctgttcttttgagccgcgtggcttcggcGgtTtccccggagtcgaacaggagggcgccaatgaggtt
cttctttatgctgtggcgatcggtattgcccagaactttgaattattgctcggcaccttgtactcgtccgtaatgacggcccagccgac
gctgtttgtgccgatatcgagcccaatggagtacttcttgtccatgggtacctttctcctctttaatgaattctgtgtgaaattgttat
ccgctcacaattgaatctatcataattgtgagcgctcacaattgtaaaggttagatctcoaactagtggcagcggctaactaagcggcc
tgctgactactcgccgatcaaaaggcattagctattaagggattgacgagggcgtatctgcgcagtaagaTGCGgcattWgttttagag
ctagaaatagcaagttaaaataaggctagtccgttatcaacttgaaaaagtggcaccgagtcggtgctttttttaattcgaaaagcgct
caacgagcaggctataggtcgacagACAGtagtggcagcggctaactaagcggcctgctgactttctcgccgatcaaaaggcattttgc
tattaagggattgacgagggcgtatctgcgcagtaagatgcgccccgcattYgttttagagctagaaatagcaagttaaaataaggcta
gtccgttatcaacttgaaaaagtggcaccgagtcggtgctttttttaattcgaaaagcctgctcaacgagcaggctataggtcgacagt
tcataggtgattgctcaggacatttctgttagaaggaatcgttttccttacttaccttacgcacaagagttccgtagctgttcaagttt
gtgtttcaactgttctcgtcgtttccgcaacaagtcctcttcagaaatgagctatgctc

The following Table 2 illustrates sgRNA sequences in a pCas9-hGFP-N/Δ plasmid.

| | | | |
|---|---|---|---|
| hGFP12-A/Δ: | sgRNA 1: | CCAGGATGG | (SEQ ID NO: 5) |
| | | GCACCAACC | |
| | sgRNA 2: | ACCAGGATG | (SEQ ID NO: 6) |
| | | GGCACCACC | |
| hGFP12-G/Δ: | sgRNA 1: | CCAGGATGG | (SEQ ID NO: 7) |
| | | GCACCAGCC | |
| | sgRNA 2: | ACCAGGATG | (SEQ ID NO: 8) |
| | | GGCACCACC | |
| hGFP12-C/Δ: | sgRNA 1: | CCAGGATGG | (SEQ ID NO: 9) |
| | | GCACCACCC | |
| | sgRNA 2: | ACCAGGATG | (SEQ ID NO: 10) |
| | | GGCACCACC | |
| hGFP12-T/Δ: | sgRNA 1: | CCAGGATGG | (SEQ ID NO: 11) |
| | | GCACCATCC | |
| | sgRNA 2: | ACCAGGATG | (SEQ ID NO: 12) |
| | | GGCACCACC | |
| hGFP13-A/Δ: | sgRNA 1: | CCAGGATGG | (SEQ ID NO: 13) |
| | | GAACCACCC | |
| | sgRNA 2: | ACCAGGATG | (SEQ ID NO: 14) |
| | | GGACCACCC | |
| hGFP13-G/Δ: | sgRNA 1: | CCAGGATGG | (SEQ ID NO: 15) |
| | | GGACCACCC | |
| | sgRNA 2: | ACCAGGATG | (SEQ ID NO: 16) |
| | | GGACCACCC | |
| hGFP13-C/Δ: | sgRNA 1: | CCAGGATGG | (SEQ ID NO: 17) |
| | | GCACCACCC | |
| | sgRNA 2: | ACCAGGATG | (SEQ ID NO: 18) |
| | | GGACCACCC | |
| hGFP13-T/Δ: | sgRNA 1: | CCAGGATGG | (SEQ ID NO: 19) |
| | | GTACCACCC | |
| | sgRNA 2: | ACCAGGATG | (SEQ ID NO: 20) |
| | | GGACCACCC | |
| hGFP16-A/Δ: | sgRNA 1: | CCAAGATGG | (SEQ ID NO: 21) |
| | | GCACCACCC | |
| | sgRNA 2: | ACCAGATGG | (SEQ ID NO: 22) |
| | | GCACCACCC | |
| hGFP16-G/Δ: | sgRNA 1: | CCAGGATGG | (SEQ ID NO: 23) |
| | | GCACCACCC | |
| | sgRNA 2: | ACCAGATGG | (SEQ ID NO: 24) |
| | | GCACCACCC | |
| hGFP16-C/Δ: | sgRNA 1: | CCACGATGG | (SEQ ID NO: 25) |
| | | GCACCACCC | |
| | sgRNA 2: | ACCAGATGG | (SEQ ID NO: 26) |
| | | GCACCACCC | |
| hGFP16-T/Δ: | sgRNA 1: | CCATGATGG | (SEQ ID NO: 27) |
| | | GCACCACCC | |
| | sgRNA 2: | ACCAGATGG | (SEQ ID NO: 28) |
| | | GCACCACCC | |

The following Table 3 illustrates sgRNA sequences used in one or more of a method, composition, cell, engineered microorganism described herein.

| | | |
|---|---|---|
| GFP151-GXC | TCACACAATGTAGXCATCACGG | (SEQ ID NO: 29) |
| GFP12-YTG | ACCAGGATGGGCACCAYCCCGG | (SEQ ID NO: 30) |
| hGFP16-YTG | ACCAYGATGGGCACCACCCCGG | (SEQ ID NO: 31) |
| GFP151-XAG | TCACACAATGTAXAGATCACGG | (SEQ ID NO: 32) |
| hGFP12-XTG | ACCAGGATGGGCACCAXCCCGG | (SEQ ID NO: 33) |
| TK1-NC-AXT | TGTTGTGTGGAAXTGTGAGCGG | (SEQ ID NO: 34) |
| GFP66-YGC | TTGTCACTACTCTGACCYGCGG | (SEQ ID NO: 35) |
| GFP66-XAG | TTGTCACTACTCTGACCXAGGG | (SEQ ID NO: 36) |
| GFP151-CXC | TCACACAATGTACXCATCACGG | (SEQ ID NO: 37) |
| hGFP16-YTG | ACCAXGATGGGCACCACCCCGG | (SEQ ID NO: 38) |
| GFP151-TXG | TCACACAATGTATXGATCACGG | (SEQ ID NO: 39) |
| GFP151-TYA | TCACACAATGTATYAATCACGG | (SEQ ID NO: 40) |
| hGFP13-GYA | ACCAGGATGGGXACCACCCCGG | (SEQ ID NO: 41) |
| D8-NC-TXT | ATTCACAATACTXTCTTTAAGG | (SEQ ID NO: 42) |

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

What is claimed is:

1. A method of increasing production of a nucleic acid sequence containing an unnatural nucleotide, comprising transforming a cell with:
one or more nucleic acids encoding a CRISPR/Cas system; and
a nucleic acid sequence comprising an unnatural nucleotide;
wherein the method is an in vivo method;
wherein the CRISPR/Cas system encodes a single guide RNA (sgRNA) comprising a crRNA-tracrRNA scaffold;
wherein a modification at the unnatural nucleotide position within the nucleic acid sequence generates a modified nucleic acid sequence; and
wherein the CRISPR/Cas system modulates replication of the modified nucleic acid sequence to increase the production of the nucleic acid sequence comprising the unnatural nucleotide.

2. The method of claim 1, wherein the modification is a substitution, a deletion, and/or an insertion.

3. The method of claim 1, wherein the sgRNA comprises a target motif that recognizes a modification at the unnatural nucleotide position within the nucleic acid sequence.

4. The method of claim 1, wherein the sgRNA further comprises a protospacer adjacent motif (PAM) recognition element.

5. The method of claim 1, wherein the CRISPR/Cas system decreases the replication rate of the modified nucleic acid sequence.

6. The method of claim 1, wherein the unnatural nucleotide comprises an unnatural base selected from

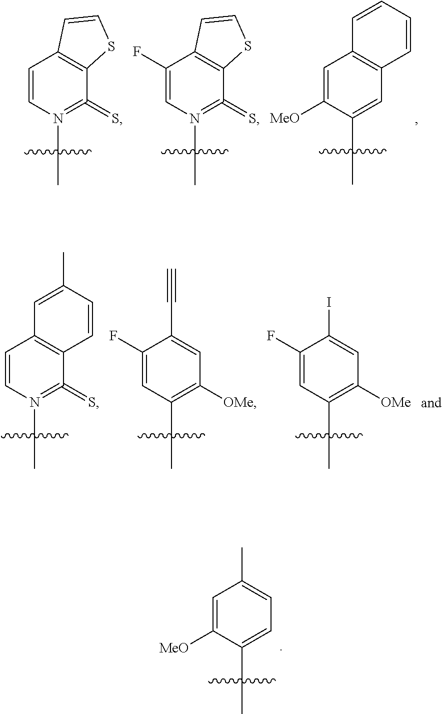

7. The method of claim 1, further comprising an additional nucleic acid sequence that encodes an additional single guide RNA (sgRNA) comprising a crRNA-tracrRNA scaffold.

8. The method of claim 1, wherein the nucleic acid sequence comprising the unnatural nucleotide further comprises an additional unnatural nucleotide.

9. The method of claim 1, wherein one or more plasmids comprise the one or more nucleic acids encoding the CRISPR/Cas system and the nucleic acid sequence comprising the unnatural nucleotide.

10. The method of claim 1, wherein the cell is E. coli.


SEQUENCE LISTING (100 sequences)

SEQ ID NO: 1 - DNA, 6363 bp, synthetic construct
  misc_feature 1..6363: note = Description of Artificial Sequence: Synthetic polynucleotide
  source 1..6363: mol_type = other DNA
ctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgagggccctgtgtgaaattgttatccgctcacaattccacacagacgtcgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagtaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcccctaggtctagggcggcggatttgtcctactcaggagagcgttcaccgacaaacaacagataaaacgaaaggcccagtctttcgactgagcctttcgttttatttgatgcctctagattacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggccgtctccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatatcgagcccaatggagtacttcttgtccatggtacctttctcctctttaatgaattctgtgtgaaattgttatccgctcacaattgaatctatcataattgtgagcgctcacaattgtaaaggttagatctaaaactagtggcagcggctaactaagcggcctgctgactttctcgccgatcaaaaggcattttgctattaagggattgacgagggcgtatctgcgcagtaagatgcgccccgcattgtatgttgtgtggaaatgtgaggttttagagctagaaatagcaagttaaaataaggctagtccgttatcaacttgaaaaagtggcaccgagtcggtgctttttttaattcgaaaagcctgctcaacgagcaggcttttttggtcgacagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctc

SEQ ID NO: 2 - DNA, 6359 bp, synthetic construct
  misc_feature 1..6359: note = Description of Artificial Sequence: Synthetic polynucleotide
  source 1..6359: mol_type = other DNA
ctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgagggccctgtgtgaaattgttatccgctcacaattccacacagacgtcgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagtaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcccctaggtctagggcggcggatttgtcctactcaggagagcgttcaccgacaaacaacagataaaacgaaaggcccagtctttcgactgagcctttcgttttatttgatgcctctagattacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggccgtctccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatatcgagcccaatggagtacttcttgtccatggtacctttctcctctttaatgaattctgtgtgaaattgttatccgctcacaattgaatctatcataattgtgagcgctcacaattgtaaaggttagatctaaaactagtggcagcggctaactaagcggcctgctgactttctcgccgatcaaaaggcattttgctattaagggattgacgagggcgtatctgcgcagtaagatgcgccccgcattgttgtgtggaaatgtgaggttttagagctagaaatagcaagttaaaataaggctagtccgttatcaacttgaaaaagtggcaccgagtcggtgctttttttaattcgaaaagcctgctcaacgagcaggcttttttggtcgacagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctc

SEQ ID NO: 3 - DNA, 6614 bp, synthetic construct
  misc_feature 1..6614: note = Description of Artificial Sequence: Synthetic polynucleotide
  source 1..6614: mol_type = other DNA
ctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgagggccctgtgtgaatttgttatccgctcacaattccacacagacgtcgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagtaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcccctaggtctagggcggcggatttgtcctactcaggagagcgttcaccgacaaacaacagataaaacgaaaggcccagtctttcgactgagcctttcgttttatttgatgcctctagattacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggcggtttccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatatcgagcccaatggagtacttcttgtccatgggtacctttctcctctttaatgaattctgtgtgaaattgttatccgctcacaattgaatctatcataattgtgagcgctcacaattgtaaaggttagatctaaaactagtggcagcggctaactaagcggcctgctgactttctcgccgatcaaaaggcattttgctattaagggattgacgagggcgtatctgcgcagtaagatgcggcattgttgtgtggaaatgtgaggttttagagctagaaatagcaagttaaaataaggctagtccgttatcaacttgaaaaagtggcaccgagtcggtgctttttttaattcgaaaagcgctcaacgagcaggcttttttggtcgacagacagtagtggcagcggctaactaagcggcctgctgactttctcgccgatcaaaaggcattttgctattaagggattgacgagggcgtatctgcgcagtaagatgcgccccgcatttgttgtgtggaatgtgaggttttagagctagaaatagcaagttaaaataaggctagtccgttatcaacttgaaaaagtggcaccgagtcggtgctttttttaattcgaaaagcctgctcaacgagcaggcttttttggtcgacagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctc

SEQ ID NO: 4 - DNA, 6614 bp, synthetic construct
  misc_feature 1..6614: note = Description of Artificial Sequence: Synthetic polynucleotide
  variation 6064: note = a, c, t or g
  variation 6071: note = a, c, t or g
  variation 6076: note = a, c, t or g
  variation 6061..6078: note = This motif may comprise a sequence selected from SEQ ID NOS 5, 7, 9, 11, 13, 15, 17, 19, 21, 23, 25, or 27
  variation 6319..6336: note = This motif may comprise a sequence selected from SEQ ID NOS 6, 8, 10, 12, 14, 16, 18, 20, 22, 24, or 26
  source 1..6614: mol_type = other DNA
ctctgcttggacggacaggatgtatgctgtggcttttttaaggataactaccttgggggccttttcattgttttccaactccgggatctggtcacgcagggcaaaaaagctccgttttagctcgttcctcctctggcgctccaagacgttgtgtgttcgcctcttgacattctcctcggtgtccgagggccctgtgtgaatttgttatccgctcacaattccacacagacgtcgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgagagctcgcttggactcctgttgatagatccagtaatgacctcagaactccatctggatttgttcagaacgctcggttgccgccgggcgttttttattggtgagaatccaagcactagtaacaacttatatcgtatggggctgacttcaggtgctacatttgaagagataaattgcactgaaatctagtaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcccctaggtctagggcggcggatttgtcctactcaggagagcgttcaccgacaaacaacagataaaacgaaaggcccagtctttcgactgagcctttcgttttatttgatgcctctagattacaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatatgatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggcggtttccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatatcgagcccaatggagtacttcttgtccatgggtacctttctcctctttaatgaattctgtgtgaaattgttatccgctcacaattgaatctatcataattgtgagcgctcacaattgtaaaggttagatctaaaactagtggcagcggctaactaagcggcctgctgactttctcgccgatcaaaaggcattttgctattaagggattgacgagggcgtatctgcgcagtaagatgcggcattccangatgggnaccanccgttttagagctagaaatagcaagttaaaataaggctagtccgttatcaacttgaaaaagtggcaccgagtcggtgctttttttaattcgaaaagcgctcaacgagcaggcttttttggtcgacagacagtagtggcagcggctaactaagcggcctgctgactttctcgccgatcaaaaggcattttgctattaagggattgacgagggcgtatctgcgcagtaagatgcgccccgcattaccagrwkggsmmcmmccgttttagagctagaaatagcaagttaaaataaggctagtccgttatcaacttgaaaaagtggcaccgagtcggtgctttttttaattcgaaaagcctgctcaacgagcaggcttttttggtcgacagttcataggtgattgctcaggacatttctgttagaaggaatcgttttccttacttttccttacgcacaagagttccgtagctgttcaagtttgtgtttcaactgttctcgtcgtttccgcaacaagtcctcttcagaaatgagcttttgctc

SEQ ID NO: 5 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccaacc

SEQ ID NO: 6 - DNA, 18 bp, synthetic construct
  misc_feature 1..18: note = Description of Combined DNA/RNA Molecule: Synthetic oligonucleotide
  source 1..18: mol_type = other DNA
accaggatgggcaccacc

SEQ ID NO: 7 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccagcc

SEQ ID NO: 8 - DNA, 18 bp, synthetic construct
  misc_feature 1..18: note = Description of Combined DNA/RNA Molecule: Synthetic oligonucleotide
  source 1..18: mol_type = other DNA
accaggatgggcaccacc

SEQ ID NO: 9 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccaccc

SEQ ID NO: 10 - DNA, 18 bp, synthetic construct
  misc_feature 1..18: note = Description of Combined DNA/RNA Molecule: Synthetic oligonucleotide
  source 1..18: mol_type = other DNA
accaggatgggcaccacc

SEQ ID NO: 11 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccatcc

SEQ ID NO: 12 - DNA, 18 bp, synthetic construct
  misc_feature 1..18: note = Description of Combined DNA/RNA Molecule: Synthetic oligonucleotide
  source 1..18: mol_type = other DNA
accaggatgggcaccacc

SEQ ID NO: 13 - DNA, 18 bp, synthetic construct
  misc_feature 1..18: note = Description of Combined DNA/RNA Molecule: Synthetic oligonucleotide
  source 1..18: mol_type = other DNA
ccaggatgggaaccaccc

SEQ ID NO: 14 - DNA, 18 bp, synthetic construct
  misc_feature 1..18: note = Description of Combined DNA/RNA Molecule: Synthetic oligonucleotide
  source 1..18: mol_type = other DNA
accaggatgggaccaccc

SEQ ID NO: 15 - DNA, 18 bp, synthetic construct
  misc_feature 1..18: note = Description of Combined DNA/RNA Molecule: Synthetic oligonucleotide
  source 1..18: mol_type = other DNA
ccaggatggggaccaccc

SEQ ID NO: 16 - DNA, 18 bp, synthetic construct
  misc_feature 1..18: note = Description of Combined DNA/RNA Molecule: Synthetic oligonucleotide
  source 1..18: mol_type = other DNA
accaggatgggaccaccc

SEQ ID NO: 17 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccaccc

SEQ ID NO: 18 - DNA, 18 bp, synthetic construct
  misc_feature 1..18: note = Description of Combined DNA/RNA Molecule: Synthetic oligonucleotide
  source 1..18: mol_type = other DNA
accaggatgggaccaccc

SEQ ID NO: 19 - DNA, 18 bp, synthetic construct
  misc_feature 1..18: note = Description of Combined DNA/RNA Molecule: Synthetic oligonucleotide
  source 1..18: mol_type = other DNA
ccaggatgggtaccaccc

SEQ ID NO: 20 - DNA, 18 bp, synthetic construct
  misc_feature 1..18: note = Description of Combined DNA/RNA Molecule: Synthetic oligonucleotide
  source 1..18: mol_type = other DNA
accaggatgggaccaccc

SEQ ID NO: 21 - DNA, 18 bp, synthetic construct
  misc_feature 1..18: note = Description of Combined DNA/RNA Molecule: Synthetic oligonucleotide
  source 1..18: mol_type = other DNA
ccaagatgggcaccaccc

SEQ ID NO: 22 - DNA, 18 bp, synthetic construct
  misc_feature 1..18: note = Description of Combined DNA/RNA Molecule: Synthetic oligonucleotide
  source 1..18: mol_type = other DNA
accagatgggcaccaccc

SEQ ID NO: 23 - DNA, 18 bp, synthetic construct
  misc_feature 1..18: note = Description of Combined DNA/RNA Molecule: Synthetic oligonucleotide
  source 1..18: mol_type = other DNA
ccaggatgggcaccaccc

SEQ ID NO: 24 - DNA, 18 bp, synthetic construct
  misc_feature 1..18: note = Description of Combined DNA/RNA Molecule: Synthetic oligonucleotide
  source 1..18: mol_type = other DNA
accagatgggcaccaccc

SEQ ID NO: 25 - DNA, 18 bp, synthetic construct
  misc_feature 1..18: note = Description of Combined DNA/RNA Molecule: Synthetic oligonucleotide
  source 1..18: mol_type = other DNA
ccacgatgggcaccaccc

SEQ ID NO: 26 - DNA, 18 bp, synthetic construct
  misc_feature 1..18: note = Description of Combined DNA/RNA Molecule: Synthetic oligonucleotide
  source 1..18: mol_type = other DNA
accagatgggcaccaccc

SEQ ID NO: 27 - DNA, 18 bp, synthetic construct
  misc_feature 1..18: note = Description of Combined DNA/RNA Molecule: Synthetic oligonucleotide
  source 1..18: mol_type = other DNA
ccatgatgggcaccaccc

SEQ ID NO: 28 - DNA, 18 bp, synthetic construct
  misc_feature 1..18: note = Description of Combined DNA/RNA Molecule: Synthetic oligonucleotide
  source 1..18: mol_type = other DNA
accagatgggcaccaccc

SEQ ID NO: 29 - DNA, 22 bp, synthetic construct
  misc_feature 1..22: note = Description of Combined DNA/RNA Molecule: Synthetic oligonucleotide
  variation 14: note = Any unnatural nucleotide
  source 1..22: mol_type = other DNA
tcacacaatgtagncatcacgg

SEQ ID NO: 30 - DNA, 22 bp, synthetic construct
  misc_feature 1..22: note = Description of Combined DNA/RNA Molecule: Synthetic oligonucleotide
  variation 17: note = Any unnatural nucleotide
  source 1..22: mol_type = other DNA
accaggatgggcaccancccgg

SEQ ID NO: 31 - DNA, 22 bp, synthetic construct
  misc_feature 1..22: note = Description of Combined DNA/RNA Molecule: Synthetic oligonucleotide
  variation 5: note = Any unnatural nucleotide
  source 1..22: mol_type = other DNA
accangatgggcaccaccccgg

SEQ ID NO: 32 - DNA, 22 bp, synthetic construct
  misc_feature 1..22: note = Description of Combined DNA/RNA Molecule: Synthetic oligonucleotide
  variation 13: note = Any unnatural nucleotide
  source 1..22: mol_type = other DNA
tcacacaatgtanagatcacgg

SEQ ID NO: 33 - DNA, 22 bp, synthetic construct
  misc_feature 1..22: note = Description of Combined DNA/RNA Molecule: Synthetic oligonucleotide
  variation 17: note = Any unnatural nucleotide
  source 1..22: mol_type = other DNA
accaggatgggcaccancccgg

SEQ ID NO: 34 - DNA, 22 bp, synthetic construct
  misc_feature 1..22: note = Description of Combined DNA/RNA Molecule: Synthetic oligonucleotide
  variation 13: note = Any unnatural nucleotide
  source 1..22: mol_type = other DNA
tgttgtgtggaantgtgagcgg

SEQ ID NO: 35 - DNA, 22 bp, synthetic construct
  misc_feature 1..22: note = Description of Combined DNA/RNA Molecule: Synthetic oligonucleotide
  variation 18: note = Any unnatural nucleotide
  source 1..22: mol_type = other DNA
ttgtcactactctgaccngcgg

SEQ ID NO: 36 - DNA, 22 bp, synthetic construct
  misc_feature 1..22: note = Description of Combined DNA/RNA Molecule: Synthetic oligonucleotide
  variation 18: note = Any unnatural nucleotide
  source 1..22: mol_type = other DNA
ttgtcactactctgaccnaggg

SEQ ID NO: 37 - DNA, 22 bp, synthetic construct
  misc_feature 1..22: note = Description of Combined DNA/RNA Molecule: Synthetic oligonucleotide
  variation 14: note = Any unnatural nucleotide
  source 1..22: mol_type = other DNA
tcacacaatgtacncatcacgg

SEQ ID NO: 38 - DNA, 22 bp, synthetic construct
  misc_feature 1..22: note = Description of Combined DNA/RNA Molecule: Synthetic oligonucleotide
  variation 5: note = Any unnatural nucleotide
  source 1..22: mol_type = other DNA
accangatgggcaccaccccgg

SEQ ID NO: 39 - DNA, 22 bp, synthetic construct
  misc_feature 1..22: note = Description of Combined DNA/RNA Molecule: Synthetic oligonucleotide
  variation 14: note = Any unnatural nucleotide
  source 1..22: mol_type = other DNA
tcacacaatgtatngatcacgg

SEQ ID NO: 40 - DNA, 22 bp, synthetic construct
  misc_feature 1..22: note = Description of Combined DNA/RNA Molecule: Synthetic oligonucleotide
  variation 14: note = Any unnatural nucleotide
  source 1..22: mol_type = other DNA
tcacacaatgtatnaatcacgg

SEQ ID NO: 41 - DNA, 22 bp, synthetic construct
  misc_feature 1..22: note = Description of Combined DNA/RNA Molecule: Synthetic oligonucleotide
  variation 12: note = Any unnatural nucleotide
  source 1..22: mol_type = other DNA
accaggatgggnaccaccccgg

SEQ ID NO: 42 - DNA, 22 bp, synthetic construct
  misc_feature 1..22: note = Description of Combined DNA/RNA Molecule: Synthetic oligonucleotide
  variation 13: note = Any unnatural nucleotide
  source 1..22: mol_type = other DNA
attcacaatactntctttaagg

SEQ ID NO: 43 - DNA, 20 bp, synthetic construct
  misc_feature 1..20: note = Description of Combined DNA/RNA Molecule: Synthetic oligonucleotide
  variation 18: note = g, a, u, c or unnatural nucleotides NaM - (2R,3S,5R)- 2-(hydroxymethyl)-5-(1-methoxynaphthalen-2-yl)tetrahydro- furan-3-ol or TPT3 - 6-((2R,4S,5R)-4-hydroxy-5 (hydroxymethyl) tetrahydrofuran-2-yl)thieno[2,3-c]pyridine-7(6H)-thione
  source 1..20: mol_type = other DNA
gaccaggatgggcaccancc

SEQ ID NO: 44 - DNA, 23 bp, synthetic construct
  misc_feature 1..23: note = Description of Artificial Sequence: Synthetic oligonucleotide
  variation 18: note = c, t, a, g or unnatural nucleotides NaM - (2R,3S,5R)- 2-(hydroxymethyl)-5-(1-methoxynaphthalen-2-yl)tetrahydro- furan-3-ol or TPT3 - 6-((2R,4S,5R)-4-hydroxy-5 (hydroxymethyl) tetrahydrofuran-2-yl)thieno[2,3-c]pyridine-7(6H)-thione
  source 1..23: mol_type = other DNA
gaccaggatgggcaccanccagg

SEQ ID NO: 45 - DNA, 20 bp, synthetic construct
  misc_feature 1..20: note = Description of Combined DNA/RNA Molecule: Synthetic oligonucleotide
  variation 13: note = g, a, u, c or unnatural nucleotides NaM - (2R,3S,5R)- 2-(hydroxymethyl)-5-(1-methoxynaphthalen-2-yl)tetrahydro- furan-3-ol or TPT3 - 6-((2R,4S,5R)-4-hydroxy-5 (hydroxymethyl) tetrahydrofuran-2-yl)thieno[2,3-c]pyridine-7(6H)-thione
  source 1..20: mol_type = other DNA
gaccaggatgggnaccaccc

SEQ ID NO: 46 - DNA, 23 bp, synthetic construct
  misc_feature 1..23: note = Description of Artificial Sequence: Synthetic oligonucleotide
  variation 13: note = c, t, a, g or unnatural nucleotides NaM - (2R,3S,5R)- 2-(hydroxymethyl)-5-(1-methoxynaphthalen-2-yl)tetrahydro- furan-3-ol or TPT3 - 6-((2R,4S,5R)-4-hydroxy-5 (hydroxymethyl) tetrahydrofuran-2-yl)thieno[2,3-c]pyridine-7(6H)-thione
  source 1..23: mol_type = other DNA
gaccaggatgggnaccacccagg

SEQ ID NO: 47 - DNA, 20 bp, synthetic construct
  misc_feature 1..20: note = Description of Combined DNA/RNA Molecule: Synthetic oligonucleotide
  source 1..20: mol_type = other DNA
gaccaggatgggcaccaacc

SEQ ID NO: 48 - DNA, 22 bp, synthetic construct
  misc_feature 1..22: note = Description of Combined DNA/RNA Molecule: Synthetic oligonucleotide
  modified_base 2: mod_base = OTHER; note = uracil
  modified_base 4: mod_base = OTHER; note = uracil
  modified_base 6..7: mod_base = OTHER; note = uracil
  modified_base 9: mod_base = OTHER; note = uracil
  modified_base 11: mod_base = OTHER; note = uracil
  variation 16: note = Unnatural nucleotide dTPT3TP - (((2R,3S,5R)-3-hydroxy-5- (7-thioxothieno [2,3-c]pyridin-6(7H)-yl)tetrahydrofuran- 2-yl)methyltetrahydrogen triphosphate)
  modified_base 17: mod_base = OTHER; note = uracil
  modified_base 19: mod_base = OTHER; note = uracil
  source 1..22: mol_type = other DNA
gtatgttgtgtggaantgtgag

SEQ ID NO: 49 - RNA, 20 nt, synthetic construct
  misc_feature 1..20: note = Description of Artificial Sequence: Synthetic oligonucleotide
  source 1..20: mol_type = other RNA
gaccaggatgggcaccaccc

SEQ ID NO: 50 - RNA, 22 nt, synthetic construct
  misc_feature 1..22: note = Description of Artificial Sequence: Synthetic oligonucleotide
  source 1..22: mol_type = other RNA
gtatgttgtgtggaaatgtgag

SEQ ID NO: 51 - RNA, 18 nt, synthetic construct
  misc_feature 1..18: note = Description of Artificial Sequence: Synthetic oligonucleotide
  source 1..18: mol_type = other RNA
gttgtgtggaaatgtgag

SEQ ID NO: 52 - RNA, 18 nt, synthetic construct
  misc_feature 1..18: note = Description of Artificial Sequence: Synthetic oligonucleotide
  source 1..18: mol_type = other RNA
tgttgtgtggaatgtgag

SEQ ID NO: 53 - DNA, 13 bp, synthetic construct
  misc_feature 1..13: note = Description of Artificial Sequence: Synthetic oligonucleotide
  source 1..13: mol_type = other DNA
  CDS 1..12
tcacatttccaca

SEQ ID NO: 54 - protein, 4 aa, synthetic construct
  REGION 1..4: note = Description of Artificial Sequence: Synthetic peptide
SHFH

SEQ ID NO: 55 - DNA, 21 bp, synthetic construct
  misc_feature 1..21: note = Description of Combined DNA/RNA Molecule: Synthetic oligonucleotide
  variation 16: note = Any unnatural nucleotide
  source 1..21: mol_type = other DNA
ccaggatgggcaccancccgg

SEQ ID NO: 56 - DNA, 18 bp, synthetic construct
  misc_feature 1..18: note = Description of Combined DNA/RNA Molecule: Synthetic oligonucleotide
  variation 16: note = a, c, u or g
  source 1..18: mol_type = other DNA
ccaggatgggcaccancc

SEQ ID NO: 57 - DNA, 21 bp, synthetic construct
  misc_feature 1..21: note = Description of Combined DNA/RNA Molecule: Synthetic oligonucleotide
  variation 11: note = Any unnatural nucleotide
  source 1..21: mol_type = other DNA
ccaggatgggnaccaccccgg

SEQ ID NO: 58 - DNA, 18 bp, synthetic construct
  misc_feature 1..18: note = Description of Combined DNA/RNA Molecule: Synthetic oligonucleotide
  variation 11: note = a, c, u or g
  source 1..18: mol_type = other DNA
ccaggatgggnaccaccc

SEQ ID NO: 59 - DNA, 21 bp, synthetic construct
  misc_feature 1..21: note = Description of Combined DNA/RNA Molecule: Synthetic oligonucleotide
  variation 4: note = Any unnatural nucleotide
  source 1..21: mol_type = other DNA
ccangatgggcaccaccccgg

SEQ ID NO: 60 - DNA, 18 bp, synthetic construct
  misc_feature 1..18: note = Description of Combined DNA/RNA Molecule: Synthetic oligonucleotide
  variation 4: note = a, c, u or g
  source 1..18: mol_type = other DNA
ccangatgggcaccaccc

SEQ ID NO: 61 - DNA, 21 bp, synthetic construct
  misc_feature 1..21: note = Description of Combined DNA/RNA Molecule: Synthetic oligonucleotide
  variation 16: note = Any unnatural nucleotide
  source 1..21: mol_type = other DNA
ccaggatgggcaccancccgg

SEQ ID NO: 62 - DNA, 21 bp, synthetic construct
  misc_feature 1..21: note = Description of Combined DNA/RNA Molecule: Synthetic oligonucleotide
  variation 11: note = Any unnatural nucleotide
  source 1..21: mol_type = other DNA
ccaggatgggnaccaccccgg

SEQ ID NO: 63 - DNA, 21 bp, synthetic construct
  misc_feature 1..21: note = Description of Combined DNA/RNA Molecule: Synthetic oligonucleotide
  variation 4: note = Any unnatural nucleotide
  source 1..21: mol_type = other DNA
ccangatgggcaccaccccgg

SEQ ID NO: 64 - DNA, 22 bp, synthetic construct
  misc_feature 1..22: note = Description of Combined DNA/RNA Molecule: Synthetic oligonucleotide
  variation 14: note = Any unnatural nucleotide
  source 1..22: mol_type = other DNA
tcacacaatgtagngatcacgg

SEQ ID NO: 65 - DNA, 22 bp, synthetic construct
  misc_feature 1..22: note = Description of Combined DNA/RNA Molecule: Synthetic oligonucleotide
  variation 12: note = Any unnatural nucleotide
  source 1..22: mol_type = other DNA
accaggatgggnaccaccccgg

SEQ ID NO: 66 - DNA, 20 bp, synthetic construct
  misc_feature 1..20: note = Description of Artificial Sequence: Synthetic oligonucleotide
  variation 18: note = a, c, t or g
  source 1..20: mol_type = other DNA
gaccaggatgggcaccancc

SEQ ID NO: 67 - DNA, 23 bp, synthetic construct
  misc_feature 1..23: note = Description of Artificial Sequence: Synthetic oligonucleotide
  variation 18: note = a, c, t or g
  source 1..23: mol_type = other DNA
gaccaggatgggcaccanccagg

SEQ ID NO: 68 - DNA, 20 bp, synthetic construct
  misc_feature 1..20: note = Description of Artificial Sequence: Synthetic oligonucleotide
  variation 13: note = a, c, t or g
  source 1..20: mol_type = other DNA
gaccaggatgggnaccaccc

SEQ ID NO: 69 - DNA, 23 bp, synthetic construct
  misc_feature 1..23: note = Description of Artificial Sequence: Synthetic oligonucleotide
  variation 13: note = a, c, t or g
  source 1..23: mol_type = other DNA
gaccaggatgggnaccacccagg

SEQ ID NO: 70 - DNA, 20 bp, synthetic construct
  misc_feature 1..20: note = Description of Artificial Sequence: Synthetic oligonucleotide
  source 1..20: mol_type = other DNA
gaccaggatgggcaccaacc

SEQ ID NO: 71 - RNA, 22 nt, unidentified
  misc_feature 1..22: note = Description of Unknown: target sequence
  source 1..22: mol_type = unassigned RNA
gtatgttgtgtggaaytgtgag

SEQ ID NO: 72 - RNA, 20 nt, synthetic construct
  misc_feature 1..20: note = Description of Artificial Sequence: Synthetic oligonucleotide
  source 1..20: mol_type = other RNA
gaccaggatgggcaccaccc

SEQ ID NO: 73 - RNA, 22 nt, synthetic construct
  misc_feature 1..22: note = Description of Artificial Sequence: Synthetic oligonucleotide
  source 1..22: mol_type = other RNA
gtatgttgtgtggaaatgtgag

SEQ ID NO: 74 - RNA, 18 nt, synthetic construct
  misc_feature 1..18: note = Description of Artificial Sequence: Synthetic oligonucleotide
  source 1..18: mol_type = other RNA
gttgtgtggaaatgtgag

SEQ ID NO: 75 - RNA, 18 nt, synthetic construct
  misc_feature 1..18: note = Description of Artificial Sequence: Synthetic oligonucleotide
  source 1..18: mol_type = other RNA
tgttgtgtggaatgtgag

SEQ ID NO: 76 - DNA, 21 bp, synthetic construct
  misc_feature 1..21: note = Description of Artificial Sequence: Synthetic oligonucleotide
  source 1..21: mol_type = other DNA
ccaggatgggcaccaycccgg

SEQ ID NO: 77 - DNA, 18 bp, synthetic construct
  misc_feature 1..18: note = Description of Artificial Sequence: Synthetic oligonucleotide
  variation 16: note = a, c, t or g
  source 1..18: mol_type = other DNA
ccaggatgggcaccancc

SEQ ID NO: 78 - DNA, 21 bp, synthetic construct
  misc_feature 1..21: note = Description of Artificial Sequence: Synthetic oligonucleotide
  source 1..21: mol_type = other DNA
ccaggatgggyaccaccccgg

SEQ ID NO: 79 - DNA, 18 bp, synthetic construct
  misc_feature 1..18: note = Description of Artificial Sequence: Synthetic oligonucleotide
  variation 11: note = a, c, t or g
  source 1..18: mol_type = other DNA
ccaggatgggnaccaccc

SEQ ID NO: 80 - DNA, 21 bp, synthetic construct
  misc_feature 1..21: note = Description of Artificial Sequence: Synthetic oligonucleotide
  source 1..21: mol_type = other DNA
ccaygatgggcaccaccccgg

SEQ ID NO: 81 - DNA, 18 bp, synthetic construct
  misc_feature 1..18: note = Description of Artificial Sequence: Synthetic oligonucleotide
  variation 4: note = a, c, t or g
  source 1..18: mol_type = other DNA
ccangatgggcaccaccc

SEQ ID NO: 82 - DNA, 21 bp, synthetic construct
  misc_feature 1..21: note = Description of Artificial Sequence: Synthetic oligonucleotide
  variation 16: note = a, c, t or g
  source 1..21: mol_type = other DNA
ccaggatgggcaccancccgg

SEQ ID NO: 83 - DNA, 21 bp, synthetic construct
  misc_feature 1..21: note = Description of Artificial Sequence: Synthetic oligonucleotide
  variation 11: note = a, c, t or g
  source 1..21: mol_type = other DNA
ccaggatgggnaccaccccgg

SEQ ID NO: 84 - DNA, 21 bp, synthetic construct
  misc_feature 1..21: note = Description of Artificial Sequence: Synthetic oligonucleotide
  variation 4: note = a, c, t or g
  source 1..21: mol_type = other DNA
ccangatgggcaccaccccgg

SEQ ID NO: 85 - DNA, 22 bp, synthetic construct
  misc_feature 1..22: note = Description of Artificial Sequence: Synthetic oligonucleotide
  variation 14: note = a, c, t or g
  source 1..22: mol_type = other DNA
tcacacaatgtagngatcacgg

SEQ ID NO: 86 - DNA, 22 bp, synthetic construct
  misc_feature 1..22: note = Description of Artificial Sequence: Synthetic oligonucleotide
  source 1..22: mol_type = other DNA
accaggatgggyaccaccccgg

SEQ ID NO: 87 - DNA, 22 bp, synthetic construct
  misc_feature 1..22: note = Description of Artificial Sequence: Synthetic oligonucleotide
  variation 14: note = a, c, t or g
  source 1..22: mol_type = other DNA
tcacacaatgtagncatcacgg

SEQ ID NO: 88 - DNA, 22 bp, synthetic construct
  misc_feature 1..22: note = Description of Artificial Sequence: Synthetic oligonucleotide
  source 1..22: mol_type = other DNA
accaggatgggcaccaycccgg

SEQ ID NO: 89 - DNA, 22 bp, synthetic construct
  misc_feature 1..22: note = Description of Artificial Sequence: Synthetic oligonucleotide
  source 1..22: mol_type = other DNA
accaygatgggcaccaccccgg

SEQ ID NO: 90 - DNA, 22 bp, synthetic construct
  misc_feature 1..22: note = Description of Artificial Sequence: Synthetic oligonucleotide
  variation 13: note = a, c, t or g
  source 1..22: mol_type = other DNA
tcacacaatgtanagatcacgg

SEQ ID NO: 91 - DNA, 22 bp, synthetic construct
  misc_feature 1..22: note = Description of Artificial Sequence: Synthetic oligonucleotide
  variation 17: note = a, c, t or g
  source 1..22: mol_type = other DNA
accaggatgggcaccancccgg

SEQ ID NO: 92 - DNA, 22 bp, synthetic construct
  misc_feature 1..22: note = Description of Artificial Sequence: Synthetic oligonucleotide
  variation 13: note = a, c, t or g
  source 1..22: mol_type = other DNA
tgttgtgtggaantgtgagcgg

SEQ ID NO: 93 - DNA, 22 bp, synthetic construct
  misc_feature 1..22: note = Description of Artificial Sequence: Synthetic oligonucleotide
  source 1..22: mol_type = other DNA
ttgtcactactctgaccygcgg

SEQ ID NO: 94 - DNA, 22 bp, synthetic construct
  misc_feature 1..22: note = Description of Artificial Sequence: Synthetic oligonucleotide
  variation 18: note = a, c, t or g
  source 1..22: mol_type = other DNA
ttgtcactactctgaccnaggg

SEQ ID NO: 95 - DNA, 22 bp, synthetic construct
  misc_feature 1..22: note = Description of Artificial Sequence: Synthetic oligonucleotide
  variation 14: note = a, c, t or g
  source 1..22: mol_type = other DNA
tcacacaatgtacncatcacgg

SEQ ID NO: 96 - DNA, 22 bp, synthetic construct
  misc_feature 1..22: note = Description of Artificial Sequence: Synthetic oligonucleotide
  variation 5: note = a, c, t or g
  source 1..22: mol_type = other DNA
accangatgggcaccaccccgg

SEQ ID NO: 97 - DNA, 22 bp, synthetic construct
  misc_feature 1..22: note = Description of Artificial Sequence: Synthetic oligonucleotide
  variation 14: note = a, c, t or g
  source 1..22: mol_type = other DNA
tcacacaatgtatngatcacgg

SEQ ID NO: 98 - DNA, 22 bp, synthetic construct
  misc_feature 1..22: note = Description of Artificial Sequence: Synthetic oligonucleotide
  source 1..22: mol_type = other DNA
tcacacaatgtatyaatcacgg

SEQ ID NO: 99 - DNA, 22 bp, synthetic construct
  misc_feature 1..22: note = Description of Artificial Sequence: Synthetic oligonucleotide
  variation 12: note = a, c, t or g
  source 1..22: mol_type = other DNA
accaggatgggnaccaccccgg

SEQ ID NO: 100 - DNA, 22 bp, synthetic construct
  misc_feature 1..22: note = Description of Artificial Sequence: Synthetic oligonucleotide
  variation 13: note = a, c, t or g
  source 1..22: mol_type = other DNA
attcacaatactntctttaagg